SEQUENCE LISTING (13 sequences)

SEQ ID NO: 1 - DNA, 762 bp, synthetic construct
  source 1..762: mol_type = other DNA; note = TFRC_G4S_IFN-a2a mRNA
atgatggtggacggcgacaacagccacgtggaaatgaagctggccgtggacgaggaagagaacgccgacaacaacaccaaggccaacgtgaccaagcctaagagatgcagcggcagcatctgctacggcacaatcgccgtgatcgtgttcttcctgatcggctttatgatcggctacctgggctactgcaagagcagtgatggacctggcgaaacaggcggaggcggaggatctggtggcggaggaagcggtggcggcggatcttgtgatctgcctcagacacacagcctgggcagcagacgaacactgatgctgctggcccagatgcggaagatcagcctgttcagctgcctgaaggaccggcacgatttcggcttccctcaagaggaattcggcaaccagttccagaaggccgagacaatccctgtgctgcacgagatgatccagcagatcttcaacctgttctccaccaaggacagcagcgccgcctgggatgagacactgctggacaagttctacaccgagctgtaccagcagctgaatgacctggaagcctgcgtgatccaaggcgtgggagtgacagagacacccctgatgaaggaagatagcatcctggccgtgcgcaagtacttccagcggatcaccctgtacctgaaagagaagaagtacagcccctgcgcctgggaagtcgtgcgggccgaaatcatgagaagcttcagcctgagcaccaacctgcaagagagcctgcggagcaaagagtga

SEQ ID NO: 2 - DNA, 1836 bp, synthetic construct
  source 1..1836: mol_type = other DNA; note = TFRC_G4S_IL-12 mRNA
atgatggtggacggcgacaacagccacgtggaaatgaagctggccgtggacgaggaagagaacgccgacaacaacaccaaggccaacgtgaccaagcctaagagatgcagcggcagcatctgctacggcacaatcgccgtgatcgtgttcttcctgatcggctttatgatcggctacctgggctactgcaagagcagtgatggacctggcgaaacaggcggaggcggaggatctggtggcggaggaagtggcggcggaggttctatttgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgataacaaagaatacgagtacagcgtggaatgccaagaggacagcgcctgtccagccgccgaagagtctctgcctatcgaagtgatggtcgacgccgtgcacaagctgaagtacgagaactacaccagcagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgtagcggaggtggtggtagcggaggcggcggaagcggcggtggtggatcaggtggtggtggctctagaaacctgccagtggctacccctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaacctccttcatcaccaacggctcttgcctggccagcagaaagacaagcttcatgatggccctgtgcctgagcagcatctacgaggacctgaagatgtaccaggtggaattcaagaccatgaacgccaagctgctgatggaccccaagcggcagatcttcctggaccagaacatgctggctgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctga

SEQ ID NO: 3 - protein, 15 aa, synthetic construct
  source 1..15: note = G4S Linker
GGGGSGGGGSGGGGS

SEQ ID NO: 4 - protein, 15 aa, synthetic construct
  source 1..15: note = EAAAK linker
EAAAKEAAAKEAAAK

SEQ ID NO: 5 - protein, 5 aa, synthetic construct
  REGION 1..>5: note = The entire sequence of amino acids 1-5 can be repeated one or more times
  source 1..5: note = G4S Linker
GGGGS

SEQ ID NO: 6 - protein, 5 aa, synthetic construct
  REGION 1..>5: note = The entire sequence of amino acids 1-5 can be repeated one or more times
  source 1..5: note = EAAAK linker
EAAAK

SEQ ID NO: 7 - protein, 221 aa, synthetic construct
  source 1..221: note = TFRC_G4S_IL-2
MMVDGDNSHVEMKLAVDEEENADNNTKANVTKPKRCSGSICYGTIAVIVFFLIGFMIGYLGYCKSSDGPGETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 191 aa, synthetic construct
  source 1..191: note = FasL_G4S_IL-2
MLKKRGNHSTGLCLLVMFFMVLVALVGLGLGMFQLFHLQKETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 611 aa, synthetic construct
  source 1..611: note = TFRC_G4S_IL-12
MMVDGDNSHVEMKLAVDEEENADNNTKANVTKPKRCSGSICYGTIAVIVFFLIGFMIGYLGYCKSSDGPGETGGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 253 aa, synthetic construct
  source 1..253: note = TFRC_G4S_IFN-a2a
MMVDGDNSHVEMKLAVDEEENADNNTKANVTKPKRCSGSICYGTIAVIVFFLIGFMIGYLGYCKSSDGPGETGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - protein, 73 aa, synthetic construct
  source 1..73: note = TFRC domain
MMVDGDNSHVEMKLAVDEEENADNNTKANVTKPKRCSGSICYGTIAVIVFFLIGFMIGYLGYCKSSDGPGETG

SEQ ID NO: 12 - protein, 43 aa, synthetic construct
  source 1..43: note = FasL domain
MLKKRGNHSTGLCLLVMFFMVLVALVGLGLGMFQLFHLQKETG

SEQ ID NO: 13 - DNA, 723 bp, synthetic construct
  source 1..723: mol_type = other DNA; note = membrane-bound IL-7 mRNA
atgatggtggacggcgacaacagccacgtggagatgaagctggccgtggacgaggaggagaacgccgacaacaacaccaaggccaacgtgaccaagcccaagcgctgcagcggcagcatctgctacggcaccatcgccgtgatcgtgttcttcctgatcggcttcatgatcggctacctgggctactgcaagagcagcgacggccccggcgagaccggcggaggcggcggaagcggcggcggaggcagcggcggaggcggaagcgactgcgacatcgagggcaaggacggcaagcagtacgagagcgtgctgatggtgagcatcgaccagctgctggacagcatgaaggagatcggcagcaactgcctgaacaacgagttcaacttcttcaagcgccacatctgcgacgccaacaaggagggcatgttcctgttccgcgccgcccgcaagctgcgccagttcctgaagatgaacagcaccggcgacttcgacctgcacctgctgaaggtgagcgagggcaccaccatcctgctgaactgcaccggccaggtgaagggccgcaagcccgccgccctgggcgaggcccagcccaccaagagcctggaggagaacaagagcctgaaggagcagaagaagctgaacgacctgtgcttcctgaagcgcctgctgcaggagatcaagacctgctggaacaagatcctgatgggcaccaaggagcactaa

(19) United States
(12) Patent Application Publication
(10) Pub. No.: US 2025/0043244 A1
(43) Pub. Date: Feb. 6, 2025

(54) METHODS TO GENERATE ENHANCED TUMOR INFILTRATING LYMPHOCYTES THROUGH MICROFLUIDIC DELIVERY

(71) Applicant: STEMCELL TECHNOLOGIES CANADA INC., Vancouver (CA)

(72) Inventors: Michael Finnan MALONEY, Watertown, MA (US); Emrah Ilker OZAY, Watertown, MA (US); Scott McNabb LOUGHHEAD, Watertown, MA (US); Rebecca Jean SILVER, Watertown, MA (US); Ashley BRATE, Watertown, MA (US); Murillo SILVA, Watertown, MA (US); Jonathan B. GILBERT, Watertown, MA (US); Devin BRIDGEN, Watertown, MA (US); Colin Joseph THALHOFER, Portland, OR (US)

(21) Appl. No.: 18/709,331

(22) PCT Filed: Nov. 14, 2022
(86) PCT No.: PCT/US2022/079843

§ 371 Date: May 10, 2024

Related U.S. Application Data
(60) Provisional application No. 63/278,447, filed on Nov. 11, 2021.

(51) Int. Cl. C12N 5/0783; C07K 14/54; C07K 14/55; C07K 14/705

(52) U.S. Cl. CPC C12N 5/0636; C07K 14/5434; C07K 14/55; C07K 14/70532; C12N 2510/00

(57) ABSTRACT

The present application provides TILs comprising agents that enhance activity and/or proliferative capacity of the TILs, methods of manufacturing such TILs. and methods of using such modified TILs for enhancing an immune response.

### CROSS-REFERENCE TO RELATED APPLICATION

This PCT application claims the priority benefit of U.S. Provisional Application No. 63/278,447, filed Nov. 11, 2021, which is incorporated herein by reference in its entirety.

### REFERENCE TO SEQUENCE LISTING SUBMITTED ELECTRONICALLY VIA EFS WEB

The content of the electronically submitted sequence listing (4821_079PC04_Seqlisting_ST26.xml; Size: 20, 165 bytes; and Date of Creation: Nov. 9, 2022) submitted in this application is incorporated herein by reference in its entirety.

### FIELD OF THE INVENTION

The present disclosure relates generally to TILs comprising agents that enhance activity and/or proliferative capacity of the TILs, methods of manufacturing such TILs, and methods of using such modified TILs for enhancing an immune response.

### BACKGROUND OF THE INVENTION

There are various challenges associated with the development of Tumor Infiltrating Lymphocyte (TIL) therapies. While TIL therapies have shown significant solid tumor activity in patients, current TIL compositions require patient lymphodepletion and maintenance in high dose IL-2 after cell infusion to support clinical activity. Ex vivo engineering of the TIL product with mRNA could enhance potency, expand the potential patient population, and potentially allow for repeat dosing and concomitant treatment with other therapies.

All references cited herein, including patent applications and publications, are incorporated by reference in their entirety. The patent publications WO 2016070136, US 20180142198, WO 2017008063, US20180201889, WO 2019178005, and WO 2019178006 and WO 2020176789 are hereby expressly incorporated by reference in their entirety.

### BRIEF SUMMARY OF THE INVENTION

Provided herein is a method of increasing Granzyme B expression on tumor-infiltrating lymphocytes (TILs), comprising: (a) modifying the TILs to increase expression of: (i) a co-stimulatory molecule, (ii) a cytokine, or (ii) both a co-stimulatory molecule and a cytokine; and (b) culturing the TILs in the absence of an exogenous cytokine, wherein after the modifying and the culturing, the TILs exhibit increased Granzyme B expression upon activation, as compared to that of corresponding TILs that have not been modified (reference TILs).

Present disclosure further provides a method of increasing Granzyme B expression on tumor-infiltrating lymphocytes (TILs), comprising culturing the TILs in the absence of exogenous cytokine, wherein the TILs have been modified to increase expression of: (i) a co-stimulatory molecule, (ii) a cytokine, or (iii) both a co-stimulatory molecule and a cytokine, and wherein after the culturing, the TILs exhibit increased proliferation upon activation, as compared to that of corresponding TILs that have not been modified (reference TILs).

In some aspects, the Granzyme B expression is increased by at least about 1.5-fold, at least about 2-fold, at least about 2.5-fold, at least about 3-fold, at least about 3.5 fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, as compared to that of the reference TILs.

In some aspects, the culturing is for at least about one day, at least about two days, or at least about three days. In some aspects, the co-stimulatory molecule is CD86. In some aspects, the cytokine comprises a membrane-bound IL-2, membrane-bound IL-12, or both.

In some aspects, the modifying comprises passing a cell suspension comprising the TILs through a cell-deforming constriction, thereby causing perturbations of the TILs such that a nucleic acid encoding the co-stimulatory molecule and/or a nucleic acid encoding the cytokine enters the TILs through the perturbations when contacted with the TILs. In some aspects, the modifying further comprises contacting the TILs with the nucleic acid encoding the co-stimulatory molecule and/or the nucleic acid encoding the cytokine.

In some aspects, the TILs were modified by passing a cell suspension comprising input TILs through a cell-deforming constriction, thereby causing a perturbation of the inputs TILs such that a nucleic acid encoding the co-stimulatory molecule and/or a nucleic acid encoding the cytokine entered the input TILs when contacted with the TILs, wherein after the nucleic acid encoding the co-stimulatory molecule and/or the nucleic acid encoding the cytokine, the input TILs exhibit increased expression of the co-stimulatory molecule and/or cytokine to become the TILs that have been modified.

In some aspects, the above method further comprises contacting the cell suspension with the nucleic acid encoding the co-stimulatory molecule and/or the nucleic acid encoding the cytokine. In some aspects, the cell suspension is contacted with the nucleic acid encoding the co-stimulatory molecule and/or the nucleic acid encoding the cytokine prior to the passing of the cell suspension through the cell-deforming constriction. In some aspects, the cell suspension is contacted with the nucleic acid encoding the co-stimulatory molecule and/or the nucleic acid encoding the cytokine during the passing of the cell suspension through the cell-deforming constriction. In some aspects, the cell suspension is contacted with the nucleic acid encoding the co-stimulatory molecule and/or the nucleic acid encoding the cytokine during the passing of the cell suspension through the cell-deforming constriction.

In some aspects, the nucleic acid encoding the co-stimulatory molecule and/or the nucleic acid encoding the cytokine is mRNA.

In some aspects, the cell-deforming constriction comprises a width, which is about 10% to about 99% of the mean diameter of the TILs. In some aspects, the cell-deforming constriction comprises a width, which is about 10% to about 99% of the mean diameter of the input TILs. In some aspects, the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm. In some aspects, the width of the constriction is about 3 μm to about 5 μm. In some aspects, the width of the constriction is about 4 μm.

In some aspects, the invention provides a method of modulating the activity and/or proliferative capacity of tumor-infiltrating lymphocytes (TILs), wherein the TILs are modified to increase expression of one or more co-stimulatory molecules and/or one or more cytokines. In some aspects, the invention provides, a method of modulating the activity and/or proliferative capacity of tumor-infiltrating lymphocytes (TILs), wherein the TILs are modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the co-stimulatory molecule is CD86. In some aspects, the invention provides a method of modulating the phenotype and/or proliferative capacity of TILs, wherein the TILs are modified to increase expression of one or more cytokines.

In some embodiments of the methods described herein, the TILs are modified to comprise a chimeric membrane-bound cytokine. In some embodiments, the chimeric membrane-bound cytokine is a fusion protein comprising the cytokine and a transmembrane domain. In some embodiments, the cytokine is joined to the transmembrane domain by a peptide linker. In some embodiments, the peptide linker is (G4S) 3 (SEQ ID NO: 3) or (EAAAK) 3 (SEQ ID NO: 4). In some embodiments, the cytokine is a Type I cytokine. In some embodiments, the cytokine is IL-15, IL-12, IL-2, IFN α, IFN β, or IL-21 or functional variant thereof. In some embodiments, the cytokine is IL-2 or a functional variant thereof and/or IL-12 or a functional variant thereof. In some embodiments, the chimeric membrane-bound cytokine comprises the amino acid sequence of SEQ ID NOs: 7-10.

In some embodiments of the invention, the modified TILs comprise increased expression of one or more cytokines and/or one or more of co-stimulatory molecules, wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some embodiments of the invention, the modified TILs comprises increased expression of one or more cytokines and/or one or more of co-stimulatory molecules, wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some embodiments, the method comprises: (a) incubating the TILs with the nucleic acid encoding the chimeric membrane-bound cytokine before, during and/or after passing the cell suspension through the cell-deforming constriction (b) incubating the TILs with the nucleic acid encoding the chimeric membrane-bound cytokine and the co-stimulatory molecule before, during and/or after passing the cell suspension through the cell-deforming constriction; (c) incubating the TILs with the nucleic acid encoding the chimeric membrane-bound cytokine and the nucleic acid encoding the co-stimulatory molecule before, during and/or after passing the cell suspension through the cell-deforming constriction; or (d) incubating the TILs with the nucleic acid encoding the chimeric membrane-bound cytokine and the nucleic acid encoding the one or more co-stimulatory molecules before, during and/or after passing the cell suspension through the cell-deforming constriction. In some embodiments, the method comprises: (a) incubating the TILs with the nucleic acid encoding the chimeric membrane-bound cytokine before passing the cell suspension through the cell-deforming constriction (b) incubating the TILs with the nucleic acid encoding the chimeric membrane-bound cytokine and the co-stimulatory molecule before passing the cell suspension through the cell-deforming constriction; (c) incubating the TILs with the nucleic acid encoding the chimeric membrane-bound cytokine and the nucleic acid encoding the co-stimulatory molecule before passing the cell suspension through the cell-deforming constriction; or (d) incubating the TILs with the nucleic acid encoding the chimeric membrane-bound cytokine and the nucleic acid encoding the one or more co-stimulatory molecules before passing the cell suspension through the cell-deforming constriction. In some embodiments, one or more of the nucleic acids is mRNA.

In some embodiments of the invention, the modified TILs have increased expression of one or more of T-bet, EOMES, TCF1, and CD62L compared to corresponding TILs that are not modified. In some embodiments, the modified TILs have increased expression of one or more of T-bet, EOMES, TCF1, and CD62L when cultured in the absence of exogenous cytokines, as compared to corresponding TILs that are not modified. In some embodiments, the expression of one or more of T-bet, EOMES, TCF1, and CD62L in the modified TILs is increased by more than about 1.2-fold, 1.5-fold, 1.8-fold, 2-fold, 3-fold, 4-fold, 5-fold, 8-fold, or more than 10-fold as compared to corresponding TILs that are not modified.

In some embodiments of the invention, the modified TILs have increased expression of Granzyme B compared to corresponding TILs that are not modified. In some embodiments, the modified TILs have increased expression of Granzyme B when cultured in the absence of exogenous cytokines, as compared to corresponding TILs that are not modified. In some embodiments, the modified TILs have increased expression of Granzyme B when co-cultured with tumor cells in the absence of exogenous cytokines, as compared to corresponding TILs that are not modified. In some embodiments, the expression of Granzyme B in the modified TILs is increased by about 1.2-fold, 1.5-fold, 1.8-fold, 2-fold, 3-fold, 4-fold, 5-fold, 8-fold, or 10-fold or more as compared to corresponding TILs that are not modified.

In some embodiments, the modified TILs exhibit increased proliferation compared to corresponding TILs that are not modified. In some embodiments, the modified TILs exhibit increased proliferation when cultured in media not comprising exogenous cytokines, as compared to corresponding TILs that are not modified. In some embodiments, the exogenous cytokine is IL-2 and/or IL-12; optionally wherein the exogenous cytokine is IL-2. In some embodiments, the modified TILs exhibit increased proliferation when co-cultured with tumor cells in media not comprising exogenous cytokines, as compared to corresponding TILs that are not modified. In some embodiments, the tumor is autologous tumor.

In some aspects, the invention provides a composition comprising modified TILs modified to increase expression of one or more of co-stimulatory molecules and/or one or more cytokines. In some aspects, the invention provides a composition comprising modified TILs, wherein the TILs are modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the co-stimulatory molecule is CD86. In some embodiments, the TILs are modified to increase expression of one or more cytokines. In some aspects, the invention provides a composition comprising modified TILs the TILs are modified to comprise a chimeric membrane-bound cytokine.

In some embodiments of the compositions described herein, the chimeric membrane-bound cytokine is a fusion protein comprising the cytokine and a transmembrane domain. In some embodiments, the cytokine is joined to the transmembrane domain by a peptide linker. In some embodiments, the peptide linker is (G4S) 3 (SEQ ID NO: 3) or (EAAAK) 3 (SEQ ID NO: 4). In some embodiments, the cytokine is a Type I cytokine. In some embodiments, the cytokine is IL-15, IL-12, IL-2, IFN α, IFN β, or IL-21 or functional variant thereof. In some embodiments, the cytokine is IL-2 or a functional variant thereof and/or IL-12 or a functional variant thereof. In some embodiments, the chimeric membrane-bound cytokine comprises the amino acid sequence of SEQ ID NOs: 7-10.

In some embodiments of the compositions described herein, the modified TILs comprise increased expression of one or more cytokines and/or one or more of co-stimulatory molecules, wherein the modified TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some embodiments, the TILs comprises increased expression of one or more cytokines and/or one or more of co-stimulatory molecules, wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some embodiments, the process of preparing the modified TILs comprises: (a) incubating the TILs with the nucleic acid encoding the chimeric membrane-bound cytokine before, during and/or after passing the cell suspension through the cell-deforming constriction; (b) incubating the TILs with the nucleic acid encoding the chimeric membrane-bound cytokine and the co-stimulatory molecule before, during and/or after passing the cell suspension through the cell-deforming constriction; (c) incubating the TILs with the nucleic acid encoding the chimeric membrane-bound cytokine and the nucleic acid encoding the co-stimulatory molecule before, during and/or after passing the cell suspension through the cell-deforming constriction; or (d) incubating the TILs with the nucleic acid encoding the chimeric membrane-bound cytokine and the nucleic acid encoding the one or more co-stimulatory molecules before, during and/or after passing the cell suspension through the cell-deforming constriction. In some embodiments, the process of preparing the modified TILs comprises: (a) incubating the TILs with the nucleic acid encoding the chimeric membrane-bound cytokine before passing the cell suspension through the cell-deforming constriction (b) incubating the TILs with the nucleic acid encoding the chimeric membrane-bound cytokine and the co-stimulatory molecule before passing the cell suspension through the cell-deforming constriction; (c) incubating the TILs with the nucleic acid encoding the chimeric membrane-bound cytokine and the nucleic acid encoding the co-stimulatory molecule before passing the cell suspension through the cell-deforming constriction; or (d) incubating the TILs with the nucleic acid encoding the chimeric membrane-bound cytokine and the nucleic acid encoding the one or more co-stimulatory molecules before passing the cell suspension through the cell-deforming constriction. In some embodiments, one or more of the nucleic acids is mRNA.

In some embodiments of the compositions described herein, the modified TILs have increased expression of one or more of T-bet, EOMES, TCF1, and CD62L compared to corresponding TILs that are not modified. In some embodiments, the modified TILs have increased expression of one or more of T-bet, EOMES, TCF1, and CD62L when cultured in the absence of exogenous cytokines, compared to corresponding TILs that are not modified. In some embodiments, the expression of one or more of T-bet, EOMES, TCF1, and CD62L in modified TILs is increased by more than about 1.2-fold, 1.5-fold, 1.8-fold, 2-fold, 3-fold, 4-fold, 5-fold, 8-fold, or more than 10-fold compared to the corresponding TILs that are not modified.

In some embodiments of the invention, the modified TILs have increased expression of Granzyme B compared to corresponding TILs that are not modified. In some embodiments, the modified TILs have increased expression of Granzyme B when cultured in the absence of exogenous cytokines, as compared to corresponding TILs that are not modified. In some embodiments, the modified TILs have increased expression of Granzyme B when co-cultured with tumor cells in the absence of exogenous cytokines, as compared to corresponding TILs that are not modified. In some embodiments, the expression of Granzyme B in the modified TILs is increased by about 1.2-fold, 1.5-fold, 1.8-fold, 2-fold, 3-fold, 4-fold, 5-fold, 8-fold, or 10-fold or more as compared to corresponding TILs that are not modified.

In some embodiments, the modified TILs of the compositions exhibit increased proliferation compared to corresponding TILs that are not modified. In some embodiments, the modified TILs exhibit increased proliferation when cultured in media not comprising exogenous cytokines, as compared to corresponding TILs that are not modified. In some embodiments, the exogenous cytokine is IL-2 and/or IL-12; optionally wherein the exogenous cytokine is IL-2. In some embodiments, the modified TILs exhibit increased proliferation when co-cultured with tumor cells in media not comprising exogenous cytokines, as compared to corresponding TILs that are not modified. In some embodiments, the tumor is autologous tumor.

In some aspects, the invention provides a method of modulating an immune response in an individual, comprising administering the modified TILs to an individual, wherein the modified TILs are prepared according to the method described herein. In some aspects, the invention provides a method of modulating an immune response in an individual, comprising administering the composition described herein.

In some aspects, the invention provides a method for treating a cancer, an infectious disease, or a viral-associated disease in an individual, comprising administering the modified TILs to an individual, wherein the modified TILs are prepared according to the method described herein. In some embodiments, the invention provides a method for treating a cancer, an infectious disease, or a viral-associated disease in an individual, comprising administering the composition described herein. In some embodiments, the method comprises multiple administration of the modified TILs, or multiple administration of the composition. In some embodiments, the modified TILs is administered intravenously or intratumourally. In some embodiments, the individual is a human. In some embodiments, the modified TILs are administered prior to, concurrently with, or following administration of another therapy.

In some aspects, the invention provides a pharmaceutical composition for stimulating an immune response in an individual, wherein the composition comprises an effective amount of composition described herein. In some embodiments, the invention provides a pharmaceutical composition for use as a medicine, wherein the pharmaceutical composition comprises an effective amount of composition described herein. In some embodiments, the invention provides a pharmaceutical composition for treating a cancer, an infectious disease, or a viral-associated disease in an individual, wherein the pharmaceutical composition comprises an effective amount of composition described herein. In some embodiments, the composition comprises modified TILs that are administered prior to, concurrently with, or following administration of another therapy.

In some embodiments of the constriction-mediated delivery described herein, the width of the constriction is about 10% to about 99% of the mean diameter of the input TILs. In some embodiments, the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm. In some embodiments, the width of the constriction is about 3 μm to about 5 μm. In some embodiments, the width of the constriction is about 4 μm. In some embodiments, the cell suspension comprising the plurality of input TILs are passed through multiple constrictions wherein the multiple constrictions are arranged in series and/or in parallel.

In some aspects, the invention provides a kit for use in any of the methods described herein. In some aspects, the invention provides a kit comprising the composition described herein. In some embodiments, the kit further comprises one or more of buffers, diluents, filters, needles, syringes, or package inserts with instructions for administering the composition to an individual.

In some aspects, the invention provides a method of producing TILs comprising a chimeric membrane-bound cytokine, the method comprising introducing a nucleic acid encoding the chimeric membrane-bound cytokine to the TILs. In some embodiments, the TILs comprising the chimeric membrane-bound cytokine are prepared by: a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed; thereby generating TILs comprising a chimeric membrane-bound cytokine. In some embodiments, the method comprises incubating the TILs with the nucleic acid encoding the chimeric membrane-bound cytokine thereof before, during and/or after passing the cell suspension through the cell-deforming constriction. In some embodiments, the method comprises incubating the TILs with the nucleic acid encoding the chimeric membrane-bound cytokine before passing the cell suspension through the cell-deforming constriction. In some embodiments, the nucleic acid encoding the chimeric membrane-bound cytokine is an mRNA encoding the chimeric membrane-bound cytokine. In some embodiments, the width of the constriction is about 10% to about 99% of the mean diameter of the input TILs. In some embodiments, the width of the constriction is about 5 μm to about 12 μm, or about 6 μm to about 12 μm, or about 8 μm to about 11 μm, or about 9 μm to about 11 μm, or about 12 μm to about 15 μm. In some embodiments, the width of the constriction is about 10 μm. In some embodiments, the width of the constriction is about 8 μm. In some embodiments, the cell suspension comprising the plurality of input TILs are passed through multiple constrictions wherein the multiple constrictions are arranged in series and/or in parallel.

In some aspects, the invention provides the use of a pharmaceutical composition in the manufacture of a medicament for stimulating an immune response in an individual, wherein the pharmaceutical composition comprises an effective amount of composition described herein. In some aspects, the invention provides the use of a pharmaceutical composition in the manufacture of a medicament for treating a cancer, an infectious disease, or a viral-associated disease in an individual, wherein the pharmaceutical composition comprises an effective amount of composition described herein. In some embodiments, the pharmaceutical composition is formulated for multiple administration. In some embodiments, the pharmaceutical composition is administered intravenously or intratumourally. In some embodiments, the individual is a human. In some embodiments, the pharmaceutical composition is formulated for administration prior to, concurrently with, or following administration of another therapy.

### BRIEF DESCRIPTION OF THE DRAWINGS

FIG. 1A shows the viability of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”, e.g. cells were not processed by constriction mediated delivery), empty squeeze (e.g., constriction mediated delivery was performed with an empty payload), and GFP provided by constricted mediated delivery of mRNA were used as controls in these experiments.

FIG. 1B shows the GFP expression in tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA, were used as controls in these experiments.

FIG. 1C shows the membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 1D shows the membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 2A shows the mean fluorescence intensity (“MFI”) for GFP expression in tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA, were used as controls in these experiments.

FIG. 2B shows the mean fluorescence intensity (“MFI”) for membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 2C shows the mean fluorescence intensity (“MFI”) for membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIGS. 3A-3D show the viability of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 4A-4D show the proliferation of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 5A-5D show the CD39+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 6A-6D show the CD39+CD69+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 7A-7D show the CD62L+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 8A-8H show the CD69+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 9A-9D show the membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 10A-10D show membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 11A-11D show expression of both membrane-bound IL-2 and IL-12 (“mbIL-2 mbIL-12”) in tumor infiltrating lymphocytes expressing membrane-bound IL-2 (“mbIL-2”), mbIL-12, or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 12A-12D show the T-bet+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 13A-13D show the Eomes+ and TCF-1+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 14A-14B show the viability of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 14A) or 0 IU/mL (FIG. 14B) IL-2 following mRNA delivery. No contact (“NC”) and empty squeeze (e.g., constriction mediated delivery was performed with an empty payload) were used as controls in these experiments.

FIGS. 15A-15B show the proliferation of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 15A) or 0 IU/mL (FIG. 15B) IL-2 following mRNA delivery. No contact (“NC”) and empty squeeze (e.g., constriction mediated delivery was performed with an empty payload) were used as controls in these experiments.

FIGS. 16A-16B show the CD39+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 16A) or 0 IU/mL (FIG. 16B) IL-2 following mRNA delivery. No contact (“NC”) and empty squeeze (e.g., constriction mediated delivery was performed with an empty payload) were used as controls in these experiments.

FIGS. 17A-17B show the CD62L+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 17A) or 0 IU/mL (FIG. 17B) IL-2 following mRNA delivery. No contact (“NC”) and empty squeeze (e.g., constriction mediated delivery was performed with an empty payload) were used as controls in these experiments.

FIGS. 18A-18B show the CD86+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 18A) or 0 IU/mL (FIG. 18B) IL-2 following mRNA delivery. No contact (“NC”) and empty squeeze (e.g., constriction mediated delivery was performed with an empty payload) were used as controls in these experiments.

FIGS. 19A-19B show the membrane-bound IL-2 (“mbIL-2”) expression of tumor infiltrating lymphocytes expressing both mbIL-2 and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 19A) or 0 IU/mL (FIG. 19B) IL-2 following mRNA delivery. No contact (“NC”) and empty squeeze (e.g., constriction mediated delivery was performed with an empty payload) were used as controls in these experiments.

FIGS. 19C-19D show the mean fluorescence intensity (“MFI”) of membrane-bound IL-2 (“mbIL-2”) expression of tumor infiltrating lymphocytes expressing both mbIL-2 and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 19C) or 0 IU/mL (FIG. 19D) IL-2 following mRNA delivery. No contact (“NC”) and empty squeeze (e.g., constriction mediated delivery was performed with an empty payload) were used as controls in these experiments.

FIGS. 20A-20B show the membrane-bound IL-12 (“mbIL-12”) expression of tumor infiltrating lymphocytes expressing both mbIL-12 and membrane-bound IL-2 (“mbIL-2”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 20A) or 0 IU/mL (FIG. 20B) IL-2 following mRNA delivery. No contact (“NC”) and empty squeeze (e.g., constriction mediated delivery was performed with an empty payload) were used as controls in these experiments.

FIGS. 20C-20D show the mean fluorescence intensity (“MFI”) of membrane-bound IL-12 (“mbIL-12”) expression of tumor infiltrating lymphocytes expressing both mbIL-12 and membrane-bound IL-2 (“mbIL-2”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 20C) or 0 IU/mL (FIG. 20D) IL-2 following mRNA delivery. No contact (“NC”) and empty squeeze (e.g., constriction mediated delivery was performed with an empty payload) were used as controls in these experiments.

FIGS. 21A-21B show the T-bet+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL 12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 21A) or 0 IU/mL (FIG. 21B) IL-2 following mRNA delivery. No contact (“NC”) and empty squeeze (e.g., constriction mediated delivery was performed with an empty payload) were used as controls in these experiments.

FIGS. 22A-22B show the Eomes+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 22A) or 0 IU/mL (FIG. 22B) IL-2 following mRNA delivery. No contact (“NC”) and empty squeeze (e.g., constriction mediated delivery was performed with an empty payload) were used as controls in these experiments.

FIGS. 23A-23B show the Eomes+ and TCF-1+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 23A) or 0 IU/mL (FIG. 23B) IL-2 following mRNA delivery. No contact (“NC”) and empty squeeze (e.g., constriction mediated delivery was performed with an empty payload) were used as controls in these experiments.

FIG. 24A shows the percentage of viable cells over time within human tumor infiltrating lymphocytes (TILs) squeeze-loaded with the indicated total amount of mbIL-2 and mbIL-12 mRNAs or TILs squeeze-processed with an empty payload (Control TIL). FIGS. 24B-24C show the percentage of cells expressing mbIL-2 and mbIL-12 over time, respectively, within TILs squeeze-loaded with the indicated total amount of mbIL-2 and mbIL-12 mRNAs or TILs squeeze-processed with an empty payload (Control TIL). FIGS. 24D-24E show the representative expression of mbIL-2 and mbIL-12, respectively, at 1-day post-squeeze within TILs squeeze-loaded with the indicated total amount of mbIL-2 and mbIL-12 mRNAs or TILs squeeze-processed with an empty payload (Control TIL).

FIGS. 25A and 25B shows the number of number of viable CD8+ T cells and percentage of Ki67-positive cells, respectively, as measured by flow cytometry in TILs retrieved from three human melanoma patients that were either squeeze-processed with empty payload (Control TIL), with mbIL-2 mRNA, or with mbIL-2 and mbIL-12 mRNAs and co-cultured with autologous tumor cells for 3 days in the absence of exogenous cytokines; or in TILs squeeze-processed with empty payload and then co-cultured with autologous tumor cells for 3 days in the presence of exogenous IL-2 and IL-12 (Control TIL+ rhIL-2+12).

FIG. 26 shows the percentage of Granzyme B (GZMB)—positive cells as measured by flow cytometry in TILs retrieved from three human melanoma patients that were either squeeze-processed with empty payload (Control TIL), with mbIL-2 mRNA, or with mbIL-2 and mbIL-12 mRNAs and co-cultured with autologous tumor cells for 3 days in the absence of exogenous cytokines; or in TILs squeeze-processed with empty payload and then co-cultured with autologous tumor cells for 3 days in the presence of exogenous IL-2 and IL-12 (Control TIL+ rhIL-2+12).

FIGS. 27A-27B (fluorescent images) and FIGS. 27C-27D (scatter plots) show the amount of tumor killing by TILs retrieved from two human melanoma patients that were either squeeze-processed with empty payload (Control TIL), with mbIL-2 mRNA, or with mbIL-2 and mbIL-12 mRNAs and co-cultured with autologous tumor cells for 24 hours in the absence of exogenous cytokines; or by TILs squeeze-processed with empty payload and then co-cultured with autologous tumor cells for 24 hours in the presence of exogenous IL-2 and IL-12 (Control TIL+ rhIL-2+12).

### DETAILED DESCRIPTION OF THE INVENTION

In some aspects, provided are methods of modulating the activity and/or proliferative capacity of tumor-infiltrating lymphocytes (TILs), wherein the TILs are modified to increase expression of one or more co-stimulatory molecules and/or one or more cytokines. In some embodiments, provided are methods of modulating the activity and/or proliferative capacity of tumor-infiltrating lymphocytes (TILs), wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the TILs comprising the one or more cytokines and/or the one or more co-stimulatory molecules. In some aspects, provided herein is a method of modulating the activity and/or proliferative capacity of TILs, comprising intracellularly delivering one or more nucleic acids encoding one or more cytokines to the TILs. In some aspects, provided herein is a method of modulating the activity and/or proliferative capacity of TILs, comprising intracellularly delivering one or more nucleic acids encoding one or more co-stimulatory molecules to the TILs. In some aspects, provided herein is a method of modulating the activity and/or proliferative capacity of TILs, comprising intracellularly delivering (i) one or more nucleic acids encoding one or more cytokines, and (ii) one or more nucleic acids encoding one or more co-stimulatory molecules to the TILs.

Accordingly, in some aspects, provided herein is a method of modulating the activity and/or proliferative capacity of TILs, comprising passing a cell suspension comprising TILs through a cell-deforming constriction, thereby causing perturbations of the TILs such that (i) one or more nucleic acids encoding one or more cytokines, (ii) one or more nucleic acids encoding one or more co-stimulatory molecules, or (iii) both (i) and (ii) enter the TILs through the perturbations when contacted with the TILs. In some aspects, such a method can further comprise contacting the TILs with the (i) one or more nucleic acids encoding one or more cytokines, (ii) one or more nucleic acids encoding one or more co-stimulatory molecules, or (iii) both (i) and (ii). As further described herein, in some aspects, the one or more cytokines and/or one or more co-stimulatory molecules can modulate the activity and/or proliferative capacity of the TILs.

In some aspects, provided are compositions comprising modified TILs, wherein the TILs are modified to increase expression of one or more of co-stimulatory molecules and/or one or more cytokines. Accordingly, in some aspects, provided herein is a composition comprising modified TILs which exhibit increased expression of one or more co-stimulatory molecules as compared to reference TILs (e.g., corresponding TILs that have not been modified as described herein). In some aspects, provided herein is a composition comprising modified TILs which exhibit increased expression of one or more cytokines as compared to reference TILs (e.g., corresponding TILs that have not been modified as described herein). In some aspects, provided herein is a composition comprising modified TILs which exhibit increased expression of both one or more cytokines and one or more co-stimulatory molecules as compared to reference TILs (e.g., corresponding TILs that have not been modified as described herein).

In some embodiments, provided are compositions comprising modified TILs, wherein the modified TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. Accordingly, in some aspects, the modified TILs described herein have been passed through a cell-deforming constriction, wherein the cell-deforming constriction deformed the TILs thereby causing perturbations of the TILs such that one or more nucleic acids encoding one or more cytokines had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that one or more nucleic acids encoding one or more co-stimulatory molecules had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that both (i) one or more nucleic acids encoding one or more cytokines and (ii) one or more nucleic acids encoding one or more co-stimulatory molecules had entered the TILs through the perturbations when contacted with the TILs.

In some aspects, provided are methods of producing modified TILs comprising a chimeric membrane-bound cytokine, the method comprising introducing a nucleic acid encoding the chimeric membrane-bound cytokine to the TILs. In some embodiments, provided are methods of producing modified TILs comprising a chimeric membrane-bound cytokine, comprising: a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed; thereby generating the modified TILs comprising a chimeric membrane-bound cytokine.

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4th ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); POR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6th ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); POR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); (Ising Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods of the invention contemplate any one or more of these aspects of treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms. In some embodiments, an individual may be treated if they have a precancerous lesion.

As used herein, by “combination therapy” is meant that a first agent be administered in conjunction with another agent. “In conjunction with” refers to administration of one treatment modality in addition to another treatment modality, such as administration of a composition of TILs as described herein in addition to administration of an immunoconjugate as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

In the context of cancer, the term “treating” includes any or all of killing cancer cells, inhibiting growth of cancer cells, inhibiting replication of cancer cells, lessening of overall tumor burden and ameliorating one or more symptoms associated with the disease.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheet-like structure that acts as a boundary or lining. In some examples, the term refers to a surface or filter containing pores. This term is distinct from the term “cell membrane”.

The term “filter” as used herein refers to a porous article that allows selective passage through the pores. In some examples the term refers to a surface or membrane containing pores.

The term “exogenous” when used in reference to an agent, such as an antigen or an adjuvant, with relation to a cell refers to an agent outside of the cell or an agent delivered into the cell from outside the cell. The cell may or may not have the agent already present, and may or may not produce the agent after the exogenous agent has been delivered.

The term “heterogeneous” as used herein refers to something which is mixed or not uniform in structure or composition. In some examples the term refers to pores having varied sizes, shapes or distributions within a given surface.

The term “homogeneous” as used herein refers to something which is consistent or uniform in structure or composition throughout. In some examples, the term refers to pores having consistent sizes, shapes, or distribution within a given surface.

The term “homologous” as used herein refers to a molecule which is derived from the same organism. In some examples, the term refers to a nucleic acid or protein which is normally found or expressed within the given organism.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, and so forth. In another example, inhibit may refer to the act of slowing or stopping growth; for example, retarding or preventing the growth of a tumor cell.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In one exemplary example, enhancing an immune response may refer to employing an antigen and/or adjuvant to improve, boost, heighten, or otherwise increase an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth. As used herein to describe the modified TILs of the present disclosure, in some aspects, the term can refer to the one or more properties of the modified TILs that are improved or increased compared to corresponding non-modified TILs. Non-limiting examples of such properties are provided throughout the present disclosure.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In some examples, “modulate” refers to enhancing the presence or activity of a particular target. In some examples, “modulate” refers to suppressing the presence or activity of a particular target. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth.

The term “polynucleotide” or “nucleic acid” as used herein refers to a polymeric form of nucleotides of any length, either ribonucleotides or deoxyribonucleotides. Thus, this term includes, but is not limited to, single-, double—or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P-NH2), a mixed phosphorothioate-phosphodiester oligomer, or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S.

### Food and Drug Administration.

As used herein, “microfluidic systems” refers to systems in which low volumes (e.g., m\L, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain embodiments described herein, microfluidic systems are used to apply mechanical constriction to a cell suspended in a buffer, inducing perturbations in the cell (e.g., holes) that allow a payload or compound to enter the cytosol of the cell.

For any of the structural and functional characteristics described herein, methods of determining these characteristics are known in the art.

Methods of Enhancing Activity and/or Proliferation of Tumor Infiltrating Lymphocytes

T cell activation initiates an intra-cellular signaling cascade that ultimately results in proliferation, effector function, or death, depending on the intensity of the TCR signal and associated signals. To guard against premature or excessive activation, T cells have a requirement of two independent signals for full activation. Signal 1 is an antigen-specific signal provided by the binding of the TCR to antigenic peptide complexed with MHC. Signal 2 is mediated by either cytokines or the engagement of co-stimulatory molecules such as B7.1 (CD80) and B7.2 (CD86) on the antigen-presenting cell (APC). Signal 3 is mediated by inflammatory cytokines such as IL-2, IL-12 and IFN-α.

As used herein, tumor infiltrating lymphocytes (TILs) can comprise any or all lymphocytic cell populations that have invaded the tumor tissue. TILs have been described in a number of solid tumors, including breast cancer, and are emerging as an important biomarker in predicting the efficacy and outcome of treatment. In breast cancer, TILs may comprise primarily of cytotoxic (CD8+) and helper (CD4+) T cells, and a smaller proportion of B—and NK cells (Pruneri et al., Breast. 2018 February;37:207-214; Whitford et al., Eur J Cancer, 1992;28 (2-3): 350-6). Large numbers of TILs can correlate with the presence of tertiary lymphoid structures in tumors, which additionally housed the follicular helper T cells (Tfh) responsible for lymphocyte generation (Gu-Trantien et al., J Clin Invest, 2013 July; 123 (7): 2873-92).

TILs are useful therapeutically due to their increased specificity to tumor antigens. For therapeutic purposes, TILs can be isolated from tumor tissue by one or more methods, including but not limited to enzymatic digestion. After isolation from the tumor, subsets of TILs may optionally be isolated to increase purity of sub-populations. Subsequently the TILs can be expanded in one or more stages (Dudley et al., J Immunother. 2003; 26 (4): 332-342; Jin et al., J Immunother. 2012 April; 35 (3): 283-292). In some examples, TILs can be expanded from thousands of cells to billions of cells. The expanded TILs can be infused into an individual to treat one or more tumors. In some embodiments, the TILs are autologous to the individual. In some embodiments, the TILs are allogeneic to the individual.

In some embodiments, TILs can be isolated from carcinomas with mechanical dissociation, enzymatic disaggregation and/or density gradient centrifugation (Baldan et al., Br J Cancer 2015-4-28;112 (9): 1510-8; Tan and Lei, Methods Mol Biol. 2019; 1960:93-99).

Method of enhancing activity and/or proliferation of tumor infiltrating lymphocytes

In some aspects, provided are methods for modulating the activity and/or proliferation of tumor-infiltrating lymphocytes (TILs), wherein the TILs are modified to increase expression of one or more of Signal 2 and/or Signal 3 mediators.

In some aspects, provided are methods for modulating the activity and/or proliferative capacity of tumor-infiltrating lymphocytes (TILs), wherein the TILs are modified to increase expression of one or more co-stimulatory molecules and/or one or more cytokines. In some aspects, provided are methods for modulating the activity and/or proliferative capacity of TILs, wherein the TILs are modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the co-stimulatory molecule is CD86. In some embodiments, the TILs are modified to increase expression of one or more of B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the TILs are modified to increase expression of CD86.

In some embodiments, provided are methods for modulating the activity and/or proliferative capacity of TILs, wherein the method comprises expressing a nucleic acid encoding the co-stimulatory molecules in the TILs. In some embodiments, the method comprises expressing one or more nucleic acids encoding one or more of: B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the method comprises expressing one or more nucleic acids encoding CD86. In some embodiments, the nucleic acid encoding the co-stimulatory molecules is an mRNA. In some embodiments, the nucleic acid encoding CD86 is an mRNA. Accordingly, in some aspects, modifying the TILs to increase expression of one or more co-stimulatory molecules comprises intracellularly delivering one or more nucleic acids (e.g., mRNA) encoding one or more of the cytokines to the TILs.

In some aspects, provided are methods for modulating the activity and/or proliferative capacity of tumor-infiltrating lymphocytes (TILs), wherein the TILs are modified to increase expression of one or more co-stimulatory molecules and/or one or more cytokines. In some aspects, the method comprises modifying the TILs to increase expression of one or more co-stimulatory molecules as compared to that of reference TILs (e.g., corresponding TILs that have not been modified as described herein). In some aspects, the method comprises modifying the TILs to increase expression of one or more cytokines as compared to that of reference TILs (e.g., corresponding TILs that have not been modified as described herein). In some aspects, the method comprises modifying the TILs to increase expression of both one or more cytokines and one or more co-stimulatory molecules as compared to reference TILs (e.g., corresponding TILs that have not been modified as described herein).

In some aspects, provided are methods for modulating the activity and/or proliferative capacity of TILs, wherein the TILs are modified to increase expression of one or more of cytokines. In some embodiments, the cytokine is a type I cytokine. In some embodiments, the cytokine is a type 2 cytokine. In some embodiments, the cytokine comprises one or more of: IL-2, IL-15, IL-10, IL-12, IFN-α, or IL-21, or a functional variant thereof. In some embodiments, the cytokine is IL-2 or a functional variant thereof and/or IL-12 or a functional variant thereof. As used herein, the term “functional variant” refers to a variant of a polypeptide or protein (e.g., cytokine) having substantial or significant sequence identity to the polypeptide or protein and retaining at least one of the biological activities of the polypeptide or protein. A functional variant of a polypeptide or protein can be prepared by means known in the art in view of the present disclosure. A functional variant can include one or more modifications to the amino acid sequence of the polypeptide or protein. In some aspects, the modifications change one or more physicochemical properties of the polypeptide or protein, for example, by improving the thermal stability of the polypeptide or protein, altering the substrate specificity, changing the optimal pH, reduce immunogenicity, and the like. In some aspects, the modifications alter one or more of the biological activities of the polypeptide or protein, so long as they do not destroy or abolish all of the biological activities of the polypeptide or protein.

In some aspects, provided are methods for modulating the activity and/or proliferative capacity of TILs, wherein the method comprises expressing a nucleic acid encoding a chimeric membrane-bound cytokine in the TILs. In some embodiments, the method comprises expressing one or more nucleic acids encoding one or more of: chimeric membrane-bound cytokine in the TILs, wherein the cytokine is IL-2, IL-15, IL-10, IL-12, IFN-α, or IL-21. In some embodiments, the method comprises expressing one or more nucleic acids encoding chimeric membrane-bound cytokine in the TILs, wherein the cytokine is IL-2 and/or IL-12. In some embodiments, the nucleic acid encoding the chimeric membrane-bound cytokine is an mRNA encoding the chimeric membrane-bound cytokine. In some embodiments, the one or more mRNAs encoding chimeric membrane-bound cytokines are mRNAs encoding IL-2 and/or IL-12.

In some embodiments, the TIL is modified to increase expression of one or more cytokines. In some embodiments, the cytokine is IL-10, IL-15, IL-12, IL-2, IFN-α, IFN-γ, IL-21, or a functional variant thereof. In some embodiments, the cytokine is IFN-α2 or a functional variant thereof. In some embodiments, the cytokine is a variant cytokine (such as a modified cytokine), such as a chimeric membrane-bound cytokine. In some embodiments, the TIL is modified to increase expression of one or more chimeric membrane-bound cytokines (such as membrane bound IL-10, IL-15, IL-12, IL-2, IFN-α, IFN-γ, IL-21).

In some embodiments, the cytokine is modified, and the modified cytokine is a fusion protein comprising the cytokine and a transmembrane domain. In some embodiments, the cytokine is joined to the transmembrane domain by a peptide linker. In some embodiments, the transmembrane domain is a transferrin receptor protein 1 (TFRC) or a tumor necrosis factor (e.g., FasL) transmembrane domain. In some embodiments, the transmembrane domain comprises the amino acid sequence of SEQ ID NO: 11 or SEQ ID NO: 12. In some embodiments, the peptide linker is a G4S linker or an EAAAK linker. In embodiments, the G4S linker comprises any one of 2, 3, 4, 5, 6, 7, 8, 9, or 10 repeats of G4S sequence. In some embodiments, the EAAAK linker comprises any one of 2, 3, 4, 5, 6, 7, 8, 9, or 10 repeats of EAAAK sequence. In some embodiments, the peptide linker is (G₄S) 3 (SEQ ID NO: 3) or (EAAAK) 3 (SEQ ID NO: 4). In some embodiments, the chimeric membrane-bound cytokine comprises the amino acid sequence of SEQ ID NOs: 7-10. In some embodiments, the TIL is modified to comprise a nucleic acid that results in increased expression and/or secretion of the one or more cytokines. In some embodiments, the nucleic acid encoding the chimeric membrane-bound cytokine comprises the nucleotide sequence of SEQ ID NO: 1 or 2. In some embodiments, the cytokine is a Signal 3 effector in stimulating T cell activation. In some embodiments, the chimeric membrane-bound cytokine enhances the half-life of the cytokine in an individual compared to a non-membrane-bound cytokine. In some embodiments, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some embodiments, the membrane-bound cytokine in the modified TIL prolongs the spatial association of the cytokine with an antigen presented by an antigen-presenting cell, by about any one of: 1, 2, 3, 4, 6, 8, 12, 16, 20, 24, 28, 32, 36, 48, 72, 96 or more hours, compared to a TIL comprising a non-membrane-bound cytokine. In some embodiments, a TIL comprising membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some embodiments, the membrane-bound cytokine is a membrane-bound chemokine.

In some aspects, there is provided a composition for enhancing the activity of a TIL, the composition comprising a chimeric membrane-bound cytokine in the TIL. In some embodiments, the cytokine is modified, and the modified cytokine is a fusion protein comprising the cytokine and a transmembrane domain. In some embodiments, the cytokine is joined to the transmembrane domain by a peptide linker. In some embodiments, the transmembrane domain is a transferrin receptor protein 1 (TFRC) or a tumor necrosis factor (e.g., FasL) transmembrane domain. In some embodiments, the transmembrane domain comprises the amino acid sequence of SEQ ID NO: 11 or SEQ ID NO: 12. In some embodiments, the peptide linker is a G4S linker or an EAAAK linker. In embodiments, the G4S linker comprises any one of 2, 3, 4, 5, 6, 7, 8, 9, or 10 repeats of GAS sequence. In some embodiments, the EAAAK linker comprises any one of 2, 3, 4, 5, 6, 7, 8, 9, or 10 repeats of EAAAK sequence. In some embodiments, the peptide linker is (G₄S) 3 (SEQ ID NO: 3) or (EAAAK) 3 (SEQ ID NO: 4). In some embodiments, the chimeric membrane-bound cytokine comprises the amino acid sequence of any one of SEQ ID NOs: 7-10. In some embodiments, the TIL comprises a nucleic acid that results in increased expression and/or secretion of the one or more cytokines. In some embodiments, the nucleic acid encoding the chimeric membrane-bound cytokine comprises the nucleotide sequence of SEQ ID NO: 1 or 2. In some embodiments, the TIL comprises an mRNA that results in increased expression and/or secretion of the one or more cytokines. In some embodiments, the cytokine is a Signal 3 effector in stimulating T cell activation. In some embodiments, the chimeric membrane-bound cytokine enhances the half-life of the cytokine in an individual compared to a non-membrane-bound cytokine. In some embodiments, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some embodiments, the membrane-bound cytokine prolongs the spatial association of the cytokine with the antigens presented by the TIL introduced with the chimeric membrane-bound cytokine, by about any one of: 1, 2, 3, 4, 6, 8, 12, 16, 20, 24, 28, 32, 36, 48, 72, 96 or more hours, compared to a corresponding TIL comprising a non-membrane-bound cytokine. In some embodiments, a TIL comprising the membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some embodiments, the membrane-bound cytokine is a membrane-bound chemokine.

In some embodiments, provided are methods for modulating the activity and/or proliferative capacity of tumor-infiltrating lymphocytes (TILs), wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some embodiments, the nucleic acids encoding the cytokines and/or one or the nucleic acids encoding the co-stimulatory molecule are mRNAs. In some embodiments, wherein the cytokine is a membrane-bound cytokine, the nucleic acid encoding the chimeric membrane-bound cytokine is an mRNA encoding the chimeric membrane-bound cytokine.

In some embodiments, provided are methods for modulating the activity and/or proliferative capacity of tumor-infiltrating lymphocytes (TILs), wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some embodiments, the nucleic acids encoding the one or more cytokines are mRNAs. In some embodiments, wherein the cytokine is a membrane-bound cytokine, the nucleic acid encoding the chimeric membrane-bound cytokine is an mRNA encoding the chimeric membrane-bound cytokine.

In some embodiments, provided are methods for modulating the activity and/or proliferative capacity of tumor-infiltrating lymphocytes (TILs), wherein the method comprises incubating the TILs with the nucleic acid encoding the chimeric membrane-bound cytokine before, during and/or after passing the cell suspension through the cell-deforming constriction. In some embodiments, the method comprises incubating the TILs with the nucleic acid encoding the chimeric membrane-bound cytokine and the co-stimulatory molecule before, during and/or after passing the cell suspension through the cell-deforming constriction. In some embodiments, the method comprises incubating the TILs with the nucleic acid encoding the chimeric membrane-bound cytokine and the nucleic acid encoding the co-stimulatory molecule before, during and/or after passing the cell suspension through the cell-deforming constriction. In some embodiments, the method comprises incubating the TILs with the nucleic acid encoding the chimeric membrane-bound cytokine and the nucleic acid encoding the one or more co-stimulatory molecules before, during and/or after passing the cell suspension through the cell-deforming constriction. In some embodiments, the nucleic acid encoding the chimeric membrane-bound cytokine is an mRNA encoding the chimeric membrane-bound cytokine. As used herein, the term “incubating” comprises bringing at least a first component (e.g., nucleic acids encoding cytokine) and a second component (e.g., TILs) together, such that the first component and the second component are in closer physical proximity and can therefore interact (i.e., in contact).

In some embodiments, provided are methods for modulating the activity and/or proliferative capacity of tumor-infiltrating lymphocytes (TILs), wherein the method comprises incubating the TILs with the nucleic acid encoding the chimeric membrane-bound cytokine before passing the cell suspension through the cell-deforming constriction. In some embodiments, the method comprises incubating the TILs with the nucleic acid encoding the chimeric membrane-bound cytokine and the co-stimulatory molecule before passing the cell suspension through the cell-deforming constriction. In some embodiments, the method comprises incubating the TILs with the nucleic acid encoding the chimeric membrane-bound cytokine and the nucleic acid encoding the co-stimulatory molecule before passing the cell suspension through the cell-deforming constriction. In some embodiments, the method comprises incubating the TILs with the nucleic acid encoding the chimeric membrane-bound cytokine and the nucleic acid encoding the one or more co-stimulatory molecules before passing the cell suspension through the cell-deforming constriction. In some embodiments, the nucleic acid encoding the chimeric membrane-bound cytokine is an mRNA encoding the chimeric membrane-bound cytokine.

In some embodiments according to any one of the methods described herein, the TILs are mammalian TILs. In some embodiments, the TILs are monkey, mouse, dog, cat, horse, rat, sheep, goat, pig, or rabbit TILs. In some embodiments, the TILs are primate TILs. In some embodiments, the TILs are human TILs.

Compositions of Modified TILs with Enhanced Activity and/or Proliferation

In some aspects, provided are compositions comprising modified TILs with enhanced activity and/or proliferative capacity.

In some aspects, provided are compositions comprising modified TILs, wherein the TILs are modified to increase expression of one or more of Signal 2 and/or Signal 3 mediators.

In some aspects, provided are compositions comprising modified TILs, wherein the TILs are modified to increase expression of one or more co-stimulatory molecules and/or one or more cytokines. In some aspects, provided are compositions comprising modified TILs, wherein the TILs are modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the co-stimulatory molecule is CD86. In some embodiments, the TILs are modified to increase expression of one or more of B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the TILs are modified to increase expression of CD86.

In some embodiments, provided are compositions comprising modified TILs, wherein the TILs are modified to express a nucleic acid encoding the co-stimulatory molecules in the TILs. In some embodiments, the TILs are modified to express one or more nucleic acids encoding one or more of: B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112. In some embodiments, the TILs are modified to express one or more nucleic acids encoding CD86. In some embodiments, one or more of the nucleic acid is mRNA.

In some aspects, provided are compositions comprising modified TILs, wherein the TILs are modified to increase expression of one or more co-stimulatory molecules and/or one or more cytokines. In some aspects, provided are compositions comprising modified TILs, wherein the TILs are modified to increase expression of one or more of cytokines. In some embodiments, the cytokine is a type I cytokine. In some embodiments, the cytokine is a type 2 cytokine. In some embodiments, the cytokine comprises one or more of: IL-2, IL-15, IL-10, IL-12, IFN-α, or IL-21, or a functional variant thereof. In some embodiments, the cytokine is IL-2 or a functional variant thereof and/or IL-12 or a functional variant thereof.

In some aspects, provided are compositions comprising modified TILs, wherein the TILs are modified to express a nucleic acid encoding a chimeric membrane-bound cytokine in the TILs. In some embodiments, the TILs are modified to express one or more nucleic acids encoding one or more of: chimeric membrane-bound cytokine in the TILs, wherein the cytokine is IL-2, IL-15, IL-10, IL-12, IFN-α, or IL-21. In some embodiments, the TILs are modified to express one or more nucleic acids encoding chimeric membrane-bound cytokine in the TILs, wherein the cytokine is IL-2 and/or IL-12. In some embodiments, one or more of the nucleic acid is mRNA.

In some embodiments, the TIL are modified to increase expression of one or more cytokines. In some embodiments, the cytokine is IL-10, IL-15, IL-12, IL-2, IFN-α, IFN-γ, IL-21, or a functional variant thereof. In some embodiments, the cytokine is IFN-α2 or a functional variant thereof. In some embodiments, the cytokine is a variant cytokine (such as a modified cytokine), such as a chimeric membrane-bound cytokine. In some embodiments, the TIL is modified to increase expression of one or more chimeric membrane-bound cytokines (such as membrane bound IL-10, IL-15, IL-12, IL-2, IFN-α, IFN-γ, IL-21).

In some embodiments, the cytokine is modified, and the modified cytokine is a fusion protein comprising the cytokine and a transmembrane domain. In some embodiments, the cytokine is joined to the transmembrane domain by a peptide linker. In some embodiments, the transmembrane domain is a transferrin receptor protein 1 (TFRC) or a tumor necrosis factor (e.g., FasL) transmembrane domain. In some embodiments, the transmembrane domain comprises the amino acid sequence of SEQ ID NO: 11 or SEQ ID NO: 12. In some embodiments, the peptide linker is a G4S linker or an EAAAK linker. In embodiments, the G₄S linker comprises any one of 2, 3, 4, 5, 6, 7, 8, 9, or 10 repeats of G₄S sequence. In some embodiments, the EAAAK linker comprises any one of 2, 3, 4, 5, 6, 7, 8, 9, or 10 repeats of EAAAK sequence. In some embodiments, the peptide linker is (G₄S) 3 (SEQ ID NO: 3) or (EAAAK) 3 (SEQ ID NO: 4). In some embodiments, the chimeric membrane-bound cytokine comprises the amino acid sequence of SEQ ID NOs: 7-10. In some embodiments, the TIL is modified to comprise a nucleic acid that results in increased expression and/or secretion of the one or more cytokines. In some embodiments, the nucleic acid encoding the chimeric membrane-bound cytokine comprises the nucleotide sequence of SEQ ID NO: 1 or 2. In some embodiments, the TIL is modified to comprise an mRNA that results in increased expression and/or secretion of the one or more cytokines. In some embodiments, the cytokine is a Signal 3 effector in stimulating T cell activation. In some embodiments, the chimeric membrane-bound cytokine enhances the half-life of the cytokine in an individual compared to a non-membrane-bound cytokine. In some embodiments, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some embodiments, the membrane-bound cytokine in the modified TIL prolongs the spatial association of the cytokine with an antigen presented by an antigen-presenting cell, by about any one of: 1, 2, 3, 4, 6, 8, 12, 16, 20, 24, 28, 32, 36, 48, 72, 96 or more hours, compared to a TIL comprising a non-membrane-bound cytokine. In some embodiments, a TIL comprising membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some embodiments, the membrane-bound cytokine is a membrane-bound chemokine.

In some aspects, there is provided a composition for enhancing the activity of a TIL, the composition comprising a chimeric membrane-bound cytokine in the TIL. In some embodiments, the cytokine is modified, and the modified cytokine is a fusion protein comprising the cytokine and a transmembrane domain. In some embodiments, the cytokine is joined to the transmembrane domain by a peptide linker. In some embodiments, the transmembrane domain is a transferrin receptor protein 1 (TFRC) or a tumor necrosis factor (e.g., FasL) transmembrane domain. In some embodiments, the transmembrane domain comprises the amino acid sequence of SEQ ID NO: 11 or SEQ ID NO: 12. In some embodiments, the peptide linker is a G4S linker or an EAAAK linker. In embodiments, the G₄S linker comprises any one of 2, 3, 4, 5, 6, 7, 8, 9, or 10 repeats of GAS sequence. In some embodiments, the EAAAK linker comprises any one of 2, 3, 4, 5, 6, 7, 8, 9, or 10 repeats of EAAAK sequence. In some embodiments, the peptide linker is (G₄S) 3 (SEQ ID NO: 3) or (EAAAK) 3 (SEQ ID NO: 4). In some embodiments, the chimeric membrane-bound cytokine comprises the amino acid sequence of any one of SEQ ID NOs: 7-10. In some embodiments, the TIL comprises a nucleic acid that results in increased expression and/or secretion of the one or more cytokines. In some embodiments, the nucleic acid encoding the chimeric membrane-bound cytokine comprises the nucleotide sequence of SEQ ID NO: 1 or 2. In some embodiments, the cytokine is a Signal 3 effector in stimulating T cell activation. In some embodiments, the chimeric membrane-bound cytokine enhances the half-life of the cytokine in an individual compared to a non-membrane-bound cytokine. In some embodiments, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some embodiments, the membrane-bound cytokine prolongs the spatial association of the cytokine with the antigens presented by the TIL introduced with the chimeric membrane-bound cytokine, by about any one of: 1, 2, 3, 4, 6, 8, 12, 16, 20, 24, 28, 32, 36, 48, 72, 96 or more hours, compared to a corresponding TIL comprising a non-membrane-bound cytokine. In some embodiments, a TIL comprising the membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some embodiments, the membrane-bound cytokine is a membrane-bound chemokine.

In some embodiments, provided are compositions comprising modified TILs, wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some embodiments, the nucleic acids encoding the cytokines and/or one or the nucleic acids encoding the co-stimulatory molecule are mRNAs. In some embodiments, wherein the cytokine is a membrane-bound cytokine, the nucleic acid encoding the chimeric membrane-bound cytokine is an mRNA encoding the chimeric membrane-bound cytokine.

In some embodiments, provided are compositions comprising modified TILs, wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some embodiments, the nucleic acids encoding the one or more cytokines are mRNAs. In some embodiments, wherein the cytokine is a membrane-bound cytokine, the nucleic acid encoding the chimeric membrane-bound cytokine is an mRNA encoding the chimeric membrane-bound cytokine.

In some embodiments, provided are compositions comprising modified TILs, wherein the TILs are modified in a process comprising incubating the TILs with the nucleic acid encoding the chimeric membrane-bound cytokine before, during and/or after passing the cell suspension through the cell-deforming constriction. In some embodiments, the TILs are modified in a process comprising incubating the TILs with the nucleic acid encoding the chimeric membrane-bound cytokine and the co-stimulatory molecule before, during and/or after passing the cell suspension through the cell-deforming constriction. In some embodiments, the TILs are modified in a process comprising incubating the TILs with the nucleic acid encoding the chimeric membrane-bound cytokine and the nucleic acid encoding the co-stimulatory molecule before, during and/or after passing the cell suspension through the cell-deforming constriction. In some embodiments, the TILs are modified in a process comprising incubating the TILs with the nucleic acid encoding the chimeric membrane-bound cytokine and the nucleic acid encoding the one or more co-stimulatory molecules before, during and/or after passing the cell suspension through the cell-deforming constriction. In some embodiments, the nucleic acid encoding the chimeric membrane-bound cytokine is an mRNA encoding the chimeric membrane-bound cytokine.

In some embodiments, provided are compositions comprising modified TILs, wherein the TILs are modified in a process comprising incubating the TILs with the nucleic acid encoding the chimeric membrane-bound cytokine before passing the cell suspension through the cell-deforming constriction. In some embodiments, the TILs are modified in a process comprising incubating the TILs with the nucleic acid encoding the chimeric membrane-bound cytokine and the co-stimulatory molecule before passing the cell suspension through the cell-deforming constriction. In some embodiments, the TILs are modified in a process comprising incubating the TILs with the nucleic acid encoding the chimeric membrane-bound cytokine and the nucleic acid encoding the co-stimulatory molecule before passing the cell suspension through the cell-deforming constriction. In some embodiments, the TILs are modified in a process comprising incubating the TILs with the nucleic acid encoding the chimeric membrane-bound cytokine and the nucleic acid encoding the one or more co-stimulatory molecules before passing the cell suspension through the cell-deforming constriction. In some embodiments, the nucleic acid encoding the chimeric membrane-bound cytokine is an mRNA encoding the chimeric membrane-bound cytokine.

In some embodiments according to any one of the compositions described herein, the TILs are mammalian TILs. In some embodiments, the TILs are monkey, mouse, dog, cat, horse, rat, sheep, goat, pig, or rabbit TILs. In some embodiments, the TILs are primate TILs. In some embodiments, the TILs are human TILs.

Methods of Treatment, Compositions for Use as a medicine, and Use of Pharmaceutical Composition in manufacture of a medicament

In some aspects, there is provided a method of modulating an immune response in an individual, comprising administering modified TILs which exhibit increased expression of one or more cytokines and/or one or more co-stimulatory molecules. For instance, in some aspects, present disclosure provides a method of modulating an immune response in an individual in need thereof, comprising administering to the subject a modified TIL which exhibits increased expression of one or more cytokines. In some aspects, present disclosure provides a method of modulating an immune response in an individual in need thereof, comprising administering to the subject a modified TIL which exhibits increased expression of one or more co-stimulatory molecules. In some aspects, provided herein is a method of modulating an immune response in an individual in need thereof, comprising administering to the subject a modified TIL which exhibits increased expression of both (i) one or more cytokines and (ii) one or more co-stimulatory molecules. In some aspects, the modified TILs are prepared according to any one of the methods described herein. In some aspects, there is provided a method of modulating an immune response in an individual, comprising administering any one of the compositions comprising modified TILs described herein.

In some aspects, there is provided a method of treating a cancer, an infectious disease, or a viral-associated disease in an individual, comprising administering modified TILs prepared according to any one of the methods described herein. In some aspects, there is provided a method of treating a cancer, an infectious disease, or a viral-associated disease in an individual, comprising administering any one of the compositions comprising modified TILs described herein. Accordingly, some aspects of the present disclosure are related to a method of treating a cancer in a subject in need thereof, comprising administering to the subject any of the modified TILs described herein. In some aspects, provided herein is a method of treating an infectious disease in a subject in need thereof, comprising administering to the subject any of the modified TILs described herein. In some aspects, provided herein is a method of treating a viral-associated disease in a subject in need thereof, comprising administering to the subject any of the modified TILs described herein. Non-limiting examples of cancers, infectious diseases, and viral-associated diseases that can be treated with the present disclosure are provided elsewhere herein.

In some embodiments, the method comprises multiple administration of the multiple administration of the modified TILs or composition comprising modified TILs. As is apparent from the present disclosure, the modified TILs (or composition comprising the modified TILs) can be administered to the subject using any suitable routes of administration. Non-limiting examples of such administration routes include: intratumorally, orally, pulmonarily, intranasally, parenterally (intravenously, intra-arterially, intramuscularly, intraperitoneally, or subcutaneously), rectally, intralymphatically, intrathecally, periocularly or topically. In some embodiments, the modified TILs or composition comprising modified TILs are administered intravenously or intratumorally. In some embodiments, the individual is a mammal. In some embodiments, the individual is a monkey, mouse, dog, cat, horse, rat, sheep, goat, pig, or rabbit. In some embodiments, the individual is a primate. In some embodiments, the individual is human. In some embodiments, the modified TILs or the composition comprising modified TILs are administered prior to, concurrently with, or following administration of another therapy. In some embodiments, the another therapy is checkpoint therapy.

In some aspects, there is provided a pharmaceutical composition for stimulating an immune response in an individual, wherein the composition comprises an effective amount of any one of the compositions comprising modified TILs described herein. In some aspects, there is provided a pharmaceutical composition for use as a medicine, wherein the pharmaceutical composition comprises an effective amount of any one of the compositions comprising modified TILs described herein.

In some aspects, there is provided a pharmaceutical composition for treating a cancer, an infectious disease, or a viral-associated disease in an individual, wherein the pharmaceutical composition comprises an effective amount of any one of the compositions comprising modified TILs described herein.

In some aspects, provided are uses of a pharmaceutical composition in the manufacture of a medicament for stimulating an immune response in an individual, wherein the pharmaceutical composition comprises an effective amount of any one of the compositions comprising modified TILs described herein.

In some aspects, provided are uses of a pharmaceutical composition in the manufacture of a medicament for treating a cancer, an infectious disease, or a viral-associated disease in an individual, wherein the pharmaceutical composition comprises an effective amount of composition of any one of the compositions comprising modified TILs described herein.

In some aspects, there is provided a composition for use as a medicine, wherein the composition comprises an effective amount of modified TILs comprising a chimeric membrane-bound cytokine. In some aspects, provided are compositions for treating a cancer, an infectious disease, or a viral-associated disease with a composition in an individual, wherein the composition comprises an effective amount of TILs comprising a chimeric membrane-bound cytokine. In some aspects, provided are methods of treating a cancer, an infectious disease, or a viral-associated disease in an individual, comprising administering a composition comprising an effective amount of TILs comprising a chimeric membrane-bound cytokine to the individual.

In some embodiments, provided are uses of a composition comprising an effective amount of TILs comprising a chimeric membrane-bound cytokine in the manufacture of a medicament for stimulating an immune response in an individual and/or treating a cancer, an infectious disease, or a viral-associated disease in an individual.

In some embodiments according to any one of the pharmaceutical compositions, compositions for use, or uses described herein, the composition is formulated for multiple administration of the multiple administration. In some embodiments, the composition is administered intravenously or intratumorally. In some embodiments, the individual is a mammal. In some embodiments, the individual is a monkey, mouse, dog, cat, horse, rat, sheep, goat, pig, or rabbit. In some embodiments, the individual is a primate. In some embodiments, the individual is human. In some embodiments, the composition is formulated for administration prior to, concurrently with, or following administration of another therapy. In some embodiments, the another therapy is checkpoint therapy.

In some embodiments according to any one of the methods, pharmaceutical compositions, compositions for use, or uses described herein, the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some embodiments, the nucleic acids encoding the cytokines and/or one or the nucleic acids encoding the co-stimulatory molecule are mRNAs. In some embodiments, wherein the cytokine is a membrane-bound cytokine, the nucleic acid encoding the chimeric membrane-bound cytokine is an mRNA encoding the chimeric membrane-bound cytokine.

In some embodiments according to any one of the methods, pharmaceutical compositions, compositions for use, or uses described herein, the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the the one or more cytokines and/or the one or more co-stimulatory molecules. In some embodiments, the nucleic acids encoding the one or more cytokines are mRNAs. In some embodiments, wherein the cytokine is a membrane-bound cytokine, the nucleic acid encoding the chimeric membrane-bound cytokine is an mRNA encoding the chimeric membrane-bound cytokine.

### Modulation of Activity and Proliferative Capacity of TILs

In some embodiments according to any one of the methods, pharmaceutical compositions, compositions for use, or uses described herein, the modified TILs exhibit increased in vivo persistence compared to corresponding TILs that are not modified. In some embodiments, the modified TILs expressing membrane-bound IL-2 and/or IL-12 exhibit increased in vivo persistence compared to corresponding TILs not expressing membrane-bound IL-2 and/or IL-12.

In some embodiments according to any one of the methods, pharmaceutical compositions, compositions for use, or uses described herein, the modified TILs exhibit increased in vivo circulation time compared to corresponding TILs that are not modified. In some embodiments, the modified TILs expressing membrane-bound IL-2 and/or IL-12 exhibit increased in vivo circulation time compared to corresponding TILs not expressing membrane-bound IL-2 and/or IL-12.

In some embodiments according to any one of the methods, pharmaceutical compositions, compositions for use, or uses described herein, the modified TILs exhibit increased in vivo persistence in the absence of exogenous cytokines compared to corresponding TILs that are not modified. In some embodiments, the modified TILs expressing membrane-bound IL-2 and/or IL-12 exhibit increased in vivo persistence in the absence of exogenous cytokines compared to corresponding TILs not expressing membrane-bound IL-2 and/or IL-12.

In some embodiments according to any one of the methods, pharmaceutical compositions, compositions for use, or uses described herein, the modified TILs exhibit increased in vivo circulation time in the absence of exogenous cytokines in the absence of exogenous cytokines compared to corresponding TILs that are not modified. In some embodiments, the modified TILs expressing membrane-bound IL-2 and/or IL-12 exhibit increased in vivo circulation time in the absence of exogenous cytokines compared to corresponding TILs not expressing membrane-bound IL-2 and/or IL-12.

In some embodiments according to any one of the methods, pharmaceutical compositions, compositions for use, or uses described herein, the modified TILs exhibit increased in vivo persistence wherein TILs were cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 exhibit increased in vivo persistence wherein TILs were cultured in the absence of exogenous cytokines for any one of 1, 2, 3, 4, 5, 6, 7, 8, 9, or 10 days, compared to corresponding TILs not expressing membrane-bound IL-2 and/or IL-12.

In some embodiments according to any one of the methods, pharmaceutical compositions, compositions for use, or uses described herein, the modified TILs exhibit increased in vivo circulation time in the absence of exogenous cytokines wherein TILs were cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 exhibit increased in vivo circulation time wherein TILs were cultured in the absence of exogenous cytokines for any one of 1, 2, 3, 4, 5, 6, 7, 8, 9, or 10 days, compared to corresponding TILs not expressing membrane-bound IL-2 and/or IL-12. In some embodiments according to any one of the methods, pharmaceutical compositions, compositions for use, or uses described herein, the modified TILs have increased expression of one or more markers of central memory T-cells (such as but not limited to CD62L) compared to corresponding TILs that are not modified. As used herein, the term “central memory T cells” or “TCM cells” refer to memory T cells that express at least CD45RO, CCR7, CD62L, and/or CD127. Accordingly, in some aspects, the methods of modifying TILs described are useful for increasing the expression of one or more central memory T cell markers on the TILs. In some embodiments, the modified TILs expressing membrane-bound IL-2 and/or IL-12 have increased expression of one or more markers of central memory T-cells compared to corresponding TILs not expressing membrane-bound IL-2 and/or IL-12.

In some embodiments according to any one of the methods, pharmaceutical compositions, compositions for use, or uses described herein, the modified TILs have increased expression of one or more markers of central memory T-cells when cultured in the absence of exogenous cytokines, compared to corresponding TILs that are not modified. In some embodiments, the modified TILs have increased expression of one or more markers of central memory T-cells when cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 have increased expression of one or more markers of central memory T-cells when cultured in the absence of exogenous cytokines, compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the expression of one or more markers of central memory T-cells in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs have increased expression of one or more markers of T-cell self-renewal (such as but not limited to TCF1) compared to corresponding TILs that are not modified. In some embodiments, the modified TILs expressing membrane-bound IL-2 and/or IL-12 have increased expression of one or more markers of T-cell self-renewal compared to corresponding TILs not expressing membrane-bound IL-2 and/or IL-12.

In some embodiments according to any one of the methods, pharmaceutical compositions, compositions for use, or uses described herein, the modified TILs have increased expression of one or more markers of T-cell self-renewal when cultured in the absence of exogenous cytokines, compared to corresponding TILs that are not modified. In some embodiments, the modified TILs have increased expression of one or more markers of T-cell self-renewal when cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 have increased expression of one or more markers of T-cell self-renewal when cultured in the absence of exogenous cytokines, compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the expression of one or more markers of T-cell self renewal in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs have reduced expression of one or more markers of T-cell exhaustion (such as but not limited to CD39 or CD69) compared to corresponding TILs that are not modified. In some embodiments, the modified TILs expressing membrane-bound IL-2 and/or IL-12 have reduced expression of one or more markers of T-cell self-exhaustion compared to corresponding TILs not expressing membrane-bound IL-2 and/or IL-12. Non-limiting examples of exhaustion markers include: PD-1, CD39, TIM-3, TIGIT, and/or LAG-3.

In some embodiments according to any one of the methods, pharmaceutical compositions, compositions for use, or uses described herein, the modified TILs have reduced expression of one or more markers of T-cell exhaustion when cultured in the absence of exogenous cytokines, compared to corresponding TILs that are not modified. In some embodiments, the modified TILs have reduced expression of one or more markers of T-cell exhaustion when cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 have reduced expression of one or more markers of T-cell exhaustion when cultured in the absence of exogenous cytokines, compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the expression of one or more markers of T cell exhaustion in the modified TILs is reduced by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs have increased expression of one or more of T-bet, EOMES, TCF1, CD62L compared to corresponding TILs that are not modified. In some embodiments, the modified TILs expressing membrane-bound IL-2 and/or IL-12 have increased expression of one or more of T-bet, EOMES, TCF1, CD62L compared to corresponding TILs not expressing membrane-bound IL-2 and/or IL-12.

In some embodiments according to any one of the methods, pharmaceutical compositions, compositions for use, or uses described herein, the modified TILs have increased expression of one or more of T-bet, EOMES, TCF1, CD62L when cultured in the absence of exogenous cytokines, compared to corresponding TILs that are not modified. In some embodiments, the modified TILs have increased expression of one or more of T-bet, EOMES, TCF1, CD62L when cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 have increased expression of one or more of T-bet, EOMES, TCF1, CD62L when cultured in the absence of exogenous cytokines, compared to corresponding TILs not expressing membrane-bound IL-2 and/or IL-12. In some embodiments, the exogenous cytokine comprises exogenous IL-2. In some embodiments according to any one of the methods, pharmaceutical compositions, compositions for use, or uses described above, the expression of one or more of T-bet, EOMES, TCF1, CD62L in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs have decreased expression of one or more of T-bet, EOMES, CD39 and CD69 compared to corresponding TILs that are not modified. In some embodiments, the modified TILs expressing membrane-bound IL-2 and/or IL-12 have decreased expression of one or more of T-bet, EOMES, CD39 and CD69 compared to corresponding TILs not expressing membrane-bound IL-2 and/or IL-12.

In some embodiments according to any one of the methods, pharmaceutical compositions, compositions for use, or uses described herein, the modified TILs have decreased expression of one or more of T-bet, EOMES, CD39 and CD69 when cultured in the absence of exogenous cytokines, compared to corresponding TILs that are not modified. In some embodiments, the modified TILs have decreased expression of one or more of T-bet, EOMES, CD39 and CD69 when cultured in the absence of exogenous cytokines for any one of 1, 2, 3, 4, 5, 6, 7, 8, 9, or 10 days, compared to corresponding TILs that are not modified. In some embodiments, the modified TILs expressing membrane-bound IL-2 and/or IL-12 have decreased expression of one or more of T-bet, EOMES, CD39 and CD69 when cultured in the absence of exogenous cytokines, compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the expression of one or more of T-bet, EOMES, CD39 and CD69 in the modified TILs is decreased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs exhibit increase proliferation compared to corresponding TILs that are not modified. In some embodiments, the modified TILs expressing membrane-bound IL-2 and/or IL-12 exhibit increased proliferation, compared to corresponding TILs not expressing membrane-bound IL-2 and/or IL-12.

In some embodiments according to any one of the methods, pharmaceutical compositions, compositions for use, or uses described herein, the modified TILs exhibit increased proliferation when cultured in media not comprising exogenous cytokines (such as but not limited to exogenous IL-2), as compared to corresponding TILs that are not modified. In some embodiments, the modified TILs exhibit increased proliferation when cultured in media not comprising exogenous cytokines (such as but not limited to exogenous IL-2) for any one of 1, 2, 3, 4, 5, 6, 7, 8, 9, or 10 days, as compared to corresponding TILs that are not modified. In some embodiments, the modified TILs expressing membrane-bound IL-2 and/or IL-12 exhibit increased proliferation when cultured in media not comprising exogenous cytokines (such as but not limited to exogenous IL-2), compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the proliferation rate in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some embodiments according to any one of the methods, pharmaceutical compositions, compositions for use, or uses described herein, the modified TILs exhibit increase proliferation upon co-culture with tumor cells as compared to corresponding TILs that are not modified. In some embodiments, the modified TILs expressing membrane-bound IL-2 and/or IL-12 exhibit increased proliferation upon co-culture with tumor cells, as compared to corresponding TILs not expressing membrane-bound IL-2 and/or IL-12.

In some embodiments according to any one of the methods, pharmaceutical compositions, compositions for use, or uses described herein, the modified TILs exhibit increased proliferation when co-cultured with tumor cells in the absence of exogenous cytokines (such as but not limited to exogenous IL-2 and/or IL-12), as compared to corresponding TILs that are not modified. In some embodiments, the modified TILs exhibit increased proliferation co-cultured with tumor cells in the absence of exogenous cytokines (such as but not limited to exogenous IL-2 and/or IL-12) for any one of 1, 2, 3, 4, 5, 6, 7, 8, 9, or 10 days, as compared to corresponding TILs that are not modified. In some embodiments, the modified TILs expressing membrane-bound IL-2 and/or IL-12 exhibit increased proliferation when co-cultured with tumor cells in the absence of exogenous cytokines (such as but not limited to exogenous IL-2 and/or IL-12), as compared to corresponding TILs not expressing membrane-bound IL-2 and/or IL-12. In some embodiments, the exogenous cytokine comprises exogenous IL-2.

In some embodiments according to any one of the methods, pharmaceutical compositions, compositions for use, or uses described above, the proliferation rate in the modified TILs when co-cultured with tumor cells in the absence of exogenous cytokines is increased by about any one of: 10%, 25%, 50%, 75%, 100%, 1.2-fold, 1.5-fold, 1.8-fold, 2-fold, 3-fold, 4-fold, 5-fold, 8-fold, 10-fold, 20-fold, 50-fold, or 100-fold or more as compared to corresponding TILs that are not modified.

In some embodiments according to any one of the methods, pharmaceutical compositions, compositions for use, or uses described herein, the modified TILs exhibit increased expression of Granzyme B compared to corresponding TILs that are not modified. In some embodiments, the modified TILs expressing membrane-bound IL-2 and/or IL-12 exhibit increased expression of Granzyme B compared to corresponding TILs not expressing membrane-bound IL-2 and/or IL-12.

In some embodiments according to any one of the methods, pharmaceutical compositions, compositions for use, or uses described herein, the modified TILs exhibit increased expression of Granzyme B when cultured in the absence of exogenous cytokines, compared to corresponding TILs that are not modified. In some embodiments, the modified TILs expressing membrane-bound IL-2 and/or IL-12 exhibit increased expression of Granzyme B when cultured in the absence of exogenous cytokines, compared to corresponding TILs not expressing membrane-bound IL-2 and/or IL-12.

In some embodiments according to any one of the methods, pharmaceutical compositions, compositions for use, or uses described herein, the modified TILs exhibit increased expression of Granzyme B when co-cultured with tumor cells in the absence of exogenous cytokines, compared to corresponding TILs that are not modified. In some embodiments, the modified TILs expressing membrane-bound IL-2 and/or IL-12 exhibit increased expression of Granzyme B when co-cultured with tumor cells in the absence of exogenous cytokines, compared to corresponding TILs not expressing membrane-bound IL-2 and/or IL-12. In some embodiments, the tumor cells are autologous tumor cells.

In some embodiments according to any one of the methods, pharmaceutical compositions, compositions for use, or uses described above, the expression of Granzyme B in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, 10-fold, 20-fold, 50-fold, or 100-fold or more as compared to corresponding TILs that are not modified.

In some embodiments according to any one of the methods, pharmaceutical compositions, compositions for use, or uses described herein, the percentage of cells expressing Granzyme B within the modified TILs is higher than that within corresponding TILs that are not modified. In some embodiments, the percentage of cells expressing Granzyme B within the modified TILs expressing membrane-bound IL-2 and/or IL-12 is higher than that within corresponding TILs not expressing membrane-bound IL-2 and/or IL-12.

In some embodiments according to any one of the methods, pharmaceutical compositions, compositions for use, or uses described herein, the percentage of cells expressing Granzyme B within the modified TILs when cultured in the absence of exogenous cytokines is higher than that within corresponding TILs that are not modified. In some embodiments, the percentage of cells expressing Granzyme B within the modified TILs expressing membrane-bound IL-2 and/or IL-12 when cultured in the absence of exogenous cytokines is higher than that within corresponding TILs not expressing membrane-bound IL-2 and/or IL-12.

In some embodiments according to any one of the methods, pharmaceutical compositions, compositions for use, or uses described herein, the percentage of cells expressing Granzyme B within the modified TILs when co-cultured with tumor cells in the absence of exogenous cytokines is higher than that within corresponding TILs that are not modified. In some embodiments, the percentage of cells expressing Granzyme B within the modified TILs expressing membrane-bound IL-2 and/or IL-12 when co-cultured with tumor cells in the absence of exogenous cytokines is higher than that within corresponding TILs not expressing membrane-bound IL-2 and/or IL-12. In some embodiments, the tumor cells are autologous tumor cells.

In some embodiments according to any one of the methods, pharmaceutical compositions, compositions for use, or uses described above, the percentage of cells expressing Granzyme B within the modified TILs is higher than that within corresponding TILs that are not modified by about any one of: 10%, 25%, 50%, 75%, 100%, 1.2-fold, 1.5-fold, 1.8-fold, 2-fold, 3-fold, 4-fold, 5-fold, 8-fold, 10-fold, 20-fold, 50-fold, or more than 100-fold.

In some embodiments according to any one of the methods, pharmaceutical compositions, compositions for use, or uses described herein, the modified TILs exhibit increased tumor cell killing upon co-culture compared to corresponding TILs that are not modified. In some embodiments, the modified TILs expressing membrane-bound IL-2 and/or IL-12 exhibit increased tumor cell killing upon co-culture compared to corresponding TILs not expressing membrane-bound IL-2 and/or IL-12.

In some embodiments according to any one of the methods, pharmaceutical compositions, compositions for use, or uses described herein, the modified TILs exhibit increased tumor cell killing upon co-culture in the absence of exogenous cytokines, compared to corresponding TILs that are not modified. In some embodiments, the modified TILs expressing membrane-bound IL-2 and/or IL-12 exhibit increased tumor cell killing upon co-culture in the absence of exogenous cytokines, compared to corresponding TILs not expressing membrane-bound IL-2 and/or IL-12.

In some embodiments according to any one of the methods, pharmaceutical compositions, compositions for use, or uses described above, the tumor cell killing facilitated by the modified TILs upon coculture is increased by about any one of: 10%, 25%, 50%, 75%, 100%, 1.2-fold, 1.5-fold, 1.8-fold, 2-fold, 3-fold, 4-fold, 5-fold, 8-fold, 10-fold, 20-fold, 50-fold, or 100-fold or more as compared to corresponding TILs that are not modified. In some embodiments, the tumor cells are autologous tumor cells.

In some embodiments according to any one of the methods, pharmaceutical compositions, compositions for use, or uses described herein, the modified TILs exhibit comparable tumor cell killing upon co-culture in the absence of exogenous cytokines compared to corresponding TILs that are not modified upon co-culture in the presence of one or more exogenous cytokines. In some embodiments, the exogenous cytokine comprises exogenous IL-2. In some embodiments, the exogenous cytokine comprises exogenous IL-12. In some embodiments, the exogenous cytokine comprises exogenous IL-2 and/or IL-12.

In some embodiments according to any one of the methods, pharmaceutical compositions, compositions for use, or uses described above, the tumor cell killing facilitated by the modified TILs upon co-culture differs by no more than any one of: 1%, 2%, 5%, 8% 10%, 25%, 50%, 75%, 100%, 1.2-fold, 1.5-fold, 1.8-fold, or 2-fold as compared to tumor cell killing facilitated by corresponding TILs that are not modified in the presence of one or more exogenous cytokines. In some embodiments, the exogenous cytokine comprises exogenous IL-2 and/or IL-12. In some embodiments, the tumor cells are autologous tumor cells.

In some embodiments according to the methods, pharmaceutical compositions, compositions for use, or uses described herein, the TILs comprising the chimeric membrane-bound IL-2 and/or IL-12 are prepared by a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound IL-2 and/or IL-12 to pass through to form a perturbed input TILs; and b) incubating the perturbed input TILs with the nucleic acid encoding the chimeric membrane-bound IL-2 and/or IL-12 to allow the nucleic acid to enter the perturbed input TILs where the nucleic acid encoding the chimeric membrane-bound IL-2 and/or IL-12 is expressed; thereby generating TILs comprising chimeric membrane-bound IL-2 and/or IL-12.

### Constriction-Mediated Delivery

As described and demonstrated herein, method of modifying TILs provided herein comprises passing the TILs (e.g., cell suspension comprising the TILs) through a cell-deforming constriction, which causes perturbations in the TILs such that one or more nucleic acids encoding a cytokine and/or one or more nucleic acids encoding a co-stimulatory molecule enter the TILs through the perturbations when contacted with the TILs. As used herein, a “perturbation” refers to any opening in the cell membrane (e.g., of a TIL) that is not present under normal steady state conditions (e.g., no deformation force applied to the cells). Perturbation can comprise a hole, tear, cavity, aperture, pore, break, gap, perforation, or combinations thereof. As described herein, as a cell passes through a constriction (such as those of the present disclosure), it experiences various forces due to the constraining physical environment, including but not limited to mechanical deforming forces and/or shear forces that causes perturbations in the cell membrane.

In some embodiments, the width of the constriction is about 10% to about 99% of the mean diameter of the input TILs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input TILs. In some embodiments, the width of the constriction is about 5 μm to about 12 μm, about 6 μm to about 12 μm, about 8 μm to about 11 μm, about 9 μm to about 11 μm, about 9.5 μm to about 10.5 μm, about 8 μm to about 15 μm, about 10 μm to about 15 μm, or about 12 μm to about 15 μm. In some embodiments, the width of the constriction is about 10 μm. In some embodiments, the width of the constriction is about 3 μm to about 6 μm. In some embodiments, the width of the constriction is about 4.2 μm to about 6 μm. In some embodiments, the width of the constriction is about 4.2 μm to about 4.8 μm. In some embodiments, the width of the constriction is about 3 μm to about 5 μm. In some embodiments, the width of the constriction is about 3 μm to about 3.5 μm. In some embodiments, the width of the constriction is about 3.5 μm to about 4 μm. In some embodiments, the width of the constriction is about 4 μm to about 4.5 μm. In some embodiments, the width of the constriction is about 3.2 μm to about 3.8 μm. In some embodiments, the width of the constriction is about 3.8 μm to about 4.3 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some embodiments, the width of the constriction is about 3.5 μm. In some embodiments, the width of the constriction is about 4 μm. In some embodiments, the cell suspension comprising the input TILs are passed through multiple constrictions wherein the multiple constrictions are arranged in series and/or in parallel.

Constrictions Used in Generating Compositions of TILs Comprising Co-Stimulatory Molecule and/or Cytokine

In some embodiments, the invention provides compositions of TILs comprising a co-stimulatory molecule or cytokine. In some embodiments, the nucleic acids encoding co-stimulatory molecule and/or cytokine are delivered to the TILs intracellularly.

In some embodiments, the nucleic acids are introduced into the TILs by passing the cell through a constriction such that transient pores are introduced to the membrane of the cell thereby allowing the nucleic acids to enter the cell. Examples of constriction-based delivery of compounds into a cell are provided by WO 2013/059343, WO 2015/023982, WO 2016/070136, WO2017041050, WO2017008063, WO 2017/192785, WO 2017/192786, WO 2019/178005, WO 2019/178006, WO 2020/072833, WO2020154696, and WO2020176789.

In some embodiments, the nucleic acids are delivered into the TILs to produce the TILs of the invention by passing a cell suspension comprising the TILs through a constriction, wherein the constriction deforms the cells thereby causing a perturbation of the cells such that the nucleic acids enters the cells, wherein the nucleic acids are expressed. In some embodiments, the constriction is contained within a microfluidic channel. In some embodiments, multiple constrictions can be placed in parallel and/or in series within the microfluidic channel.

In some embodiments, the constriction within the microfluidic channel includes an entrance portion, a center point, and an exit portion. In some embodiments, the length, depth, and width of the constriction within the microfluidic channel can vary. In some embodiments, the width of the constriction within the microfluidic channel is a function of the diameter of the TILs. Methods to determine the diameter of TILs are known in the art; for example, high-content imaging, cell counters or flow cytometry.

In some embodiments of the constriction-based delivery of nucleic acids encoding co-stimulatory molecules or cytokines to TILs, the width of the constriction is about 2 μm to about 15 μm. In some embodiments, the width of the constriction is about 3 μm to about 10 μm. In some embodiments, the width of the constriction is about 3 μm to about 6 μm. In some embodiments, the width of the constriction is about 4.2 μm to about 6 μm. In some embodiments, the width of the constriction is about 4.2 μm to about 4.8 μm. In some embodiments, the width of the constriction is about 3 μm to about 5 μm. In some embodiments, the width of the constriction is about 3 μm to about 3.5 μm. In some embodiments, the width of the constriction is about 3.5 μm to about 4 μm. In some embodiments, the width of the constriction is about 4 μm to about 4.5 μm. In some embodiments, the width of the constriction is about 3.2 μm to about 3.8 μm. In some embodiments, the width of the constriction is about 3.8 μm to about 4.3 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some embodiments, the width of the constriction is about 3.5 μm. In some embodiments, the width of the constriction is about 4 μm.

In some embodiments of the constriction-based delivery of nucleic acids encoding co-stimulatory molecules or cytokines to TILs, the width of the constriction is about 3 μm to about 20 μm. In some embodiments, the width of the constriction is about 5 μm to about 15 μm. In some embodiments, the width of the constriction is about 8 μm to about 12 μm. In some embodiments, the width of the constriction is about 9 μm to about 11 μm. In some embodiments, the width of the constriction is about 9.5 μm to about 10.5 μm. In some embodiments, the width of the constriction is about 7 μm to about 9 μm. In some embodiments, the width of the constriction is about 8 μm to about 10 μm. In some embodiments, the width of the constriction is about 9 μm to about 11 μm. In some embodiments, the width of the constriction is about 10 μm to about 12 μm. In some embodiments, the width of the constriction is about 11 μm to about 13 μm. In some embodiments, the width of the constriction is about 5 μm to about 12 μm, about 6 μm to about 12 μm, about 8 μm to about 11 μm, about 9 μm to about 11 μm, about 9.5 μm to about 10.5 μm, about 8 μm to about 15 μm, about 10 μm to about 15 μm, or about 12 μm to about 15 μm. In some embodiments, the width of the constriction is about 9.7 μm to about 10.3 μm. In some embodiments, the width of the constriction is about or less than any one of 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, 15 μm, 15.5 μm, 16 μm, 16.5 μm, 17 μm, 17.5 μm, or 18 μm. In some embodiments, the width of the constriction is about or less than any one of 9.0 μm, 9.1 μm, 9.2 μm, 9.3 μm, 9.4 μm, 9.5 μm, 9.6 μm, 9.7 μm, 9.8 μm, 9.9μ, 10.0 μm, 10.1μ, 10.2 μm, 10.3 μm, 10.4 μm, 10.5 μm, 10.6μ m, 10.7 μm, 10.8 μm, 10.9 μm, or 11.0 μm. In some embodiments, the width of the constriction is about 10.0 μm.

In some embodiments of the invention, the composition comprises a plurality of TILs. In some embodiments, the width of the constriction is about 10% to about 99% of the mean diameter of a subpopulation of TILs having the smallest diameter within the population of TILs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of a subpopulation of TILs having the smallest diameter within the population of TILs. In some embodiments, the width of the constriction is any one of about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of TILs having the smallest diameter within the population of TILs. In some embodiments, the width of the constriction is any one of about 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of TILs having the smallest diameter within the population of TILs.

In some embodiments of the invention, the composition comprises a plurality of TILs. In some embodiments, the width of the constriction is about 10% to about 99% of the mean diameter of a subpopulation of TILs having the largest diameter within the population of TILs. In some embodiments, the width of the constriction is any one of about 10% to about 90%, about 10% to about 80%, about 10% to about 70%, about 20% to about 60%, about 40% to about 60%, about 30% to about 45%, about 15% to about 30%, about 15% to about 20%, about 20% to about 25%, about 25% to about 30%, about 20% to about 30%, about 30% to about 70%, or about 30% to about 60% of the mean diameter of a subpopulation of TILs having the largest diameter within the population of TILs. In some embodiments, the width of the constriction is any one of about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of TILs having the largest diameter within the population of TILs. In some embodiments, the width of the constriction is any one of about 5%, 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of TILs having the largest diameter within the population of TILs.

A number of parameters may influence the delivery of a compound to TILs for stimulating an immune response by the methods described herein. In some embodiments, the cell suspension is contacted with the compound before, concurrently, or after passing through the constriction. The TILs may pass through the constriction suspended in a solution that includes the compound to deliver, although the compound can be added to the cell suspension after the TILs pass through the constriction. In some embodiments, the compound to be delivered is coated on the constriction.

Examples of parameters that may influence the delivery of the compound into the TILs include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, buffer in the cell suspension, and the amount of time that the TILs recover or incubate after passing through the constrictions can affect the passage of the delivered compound into the TILs. Additional parameters influencing the delivery of the compound into the TILs can include the velocity of the TILs in the constriction, the shear rate in the constriction, the viscosity of the cell suspension, the velocity component that is perpendicular to flow velocity, and time in the constriction. In addition, multiple chips comprising channels in series and/or in parallel may impact delivery to TILs. Multiple chips in parallel may be useful to enhance throughput. Such parameters can be designed to control delivery of the compound. In some embodiments, the cell concentration ranges from about 10 to at least about 10¹² cells/mL or any concentration or range of concentrations therebetween. In some embodiments, delivery compound concentrations can range from about 10 ng/mL to about 1 g/mL or any concentration or range of concentrations therebetween. In some embodiments, delivery compound concentrations can range from about 1 μM to at least about 2 M or any concentration or range of concentrations therebetween.

In some embodiments, the concentration of co-stimulatory molecules incubated with the TILs is between about 0.01 μM and about 10 mM. For example, in some embodiments, the concentration of co-stimulatory molecules incubated with the TILs is any of less than about 0.01 μM, about 0.1 μM, about 1 μM, about 10 μM, about 100 μM, about 1 mM or about 10 mM. In some embodiments, the concentration of co-stimulatory molecules incubated with the TILs is greater than about 10 mM. In some embodiments, the concentration of co-stimulatory molecules incubated with the TILs is any of between about 0.01 μM and about 0.1 μM, between about 0.1 μM and about 1 μM, between about 1 μM and about 10 μM, between about 10 UM and about 100 μM, between about 100 μM and about 1 mM, or between 1 mM and about 10 mM. In some embodiments, the concentration of co-stimulatory molecules incubated with the TILs is between about 0.1 μM and about 1 mM. In some embodiments, the concentration of co-stimulatory molecules incubated with the TILs is between about 0.1 μM and about 10 μM. In some embodiments, the concentration of co-stimulatory molecules thereof incubated with the TILs is 1 μM.

In some embodiments, the concentration of nucleic acids encoding the cytokines and/or the co-stimulatory molecules incubated with TILs is between about 1 nM and about 1 mM. In some embodiments, the concentration of nucleic acids encoding the cytokines and/or the co-stimulatory molecules incubated with TILs is less than about 0.1 nM, about 1 nM, about 0.01 μM, about 0.1 μM, about 1 μM, about 10 μM, about 100 μM, about 1 mM or about 10 mM. In some embodiments, the concentration of nucleic acids encoding the cytokines and/or the co-stimulatory molecules incubated with TILs is greater than about 10 mM. In some embodiments, the concentration of nucleic acids encoding the cytokines and/or the co-stimulatory molecules incubated with TILs is any of between about 0.1 nM to about 1 nM, about 1 nM to about 10 nM, about 10 nM to about 100 nM, about 0.1 μM and about 1 μM, between about 1 μM and about 10 μM, between about 10 μM and about 100 μM, between about 100 μM and about 1 mM, or between 1 mM and about 10 mM. In some embodiments, the concentration of nucleic acids encoding the cytokines and/or the co-stimulatory molecules incubated with TILs is between about 10 nM and about 100 nM. In some embodiments, the concentration of nucleic acids encoding the cytokines and/or the co-stimulatory molecules incubated with TILs is between about 1 nM and about 10 nM. In some embodiments, the concentration of nucleic acids encoding the cytokines and/or the co-stimulatory molecules incubated with TILs is about 50 nM. In some embodiments, the nucleic acid is an mRNA.

In some embodiments, the TILs comprise the nucleic acids encoding the cytokines and/or the co-stimulatory molecules at a concentration between about 1 nM and about 1 mM. In some embodiments, the TILs comprises nucleic acids encoding the cytokines and/or the co-stimulatory molecules at a concentration of any of less than about 0.1 nM, about 1 nM, about 0.01 μM, about 0.1 μM, about 1 μM, about 10 μM, about 100 μM, about 1 mM or about 10 mM. In some embodiments, the TILs comprise the nucleic acids encoding the cytokines and/or the co-stimulatory molecules at a concentration of greater than about 10 mM. In some embodiments, the TILs comprise the nucleic acid encoding the cytokines and/or the co-stimulatory molecules at a concentration of any of between about 0.1 nM to about 1 nM, about 1 nM to about 10 nM, about 10 nM to about 100 nM, about 0.1 μM and about 1 μM, between about 1 μM and about 10 μM, between about 10 μM and about 100 μM, between about 100 μM and about 1 mM, or between 1 mM and about 10 mM. In some embodiments, the TILs comprise the nucleic acid encoding the the cytokines and/or the co-stimulatory molecules at a concentration between about 10 nM and about 100 nM. In some embodiments, the TILs comprise the nucleic acid encoding the the cytokines and/or the co-stimulatory molecules at a concentration between about 1 nM and about 10 nM. In some embodiments, the TILs comprise the nucleic acid encoding the cytokines and/or the co-stimulatory molecules at a concentration of about 50 nM. In some embodiments, the nucleic acid is an mRNA.

### Systems and Kits

In some aspects, the invention provides a system comprising one or more of the constriction, an TIL suspension, nucleic acids encoding the cytokines and/or the co-stimulatory molecules for use in the methods disclosed herein. The system can include any embodiment described for the methods disclosed above, including microfluidic channels or a surface having pores to provide cell-deforming constrictions, cell suspensions, cell perturbations, delivery parameters, compounds, and/or applications etc. In some embodiment, the cell-deforming constrictions are sized for delivery to TILs. In some embodiments, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for enhancing the activity and/or proliferative capacity of TILs.

Also provided are kits or articles of manufacture for use in treating individuals with a cancer or an infection. In some embodiments, the kit comprises a TIL comprising intracellularly nucleic acids encoding the cytokines and/or the co-stimulatory molecules. In some embodiments, the kit comprises one or more of the constriction, a TIL suspension, nucleic acids encoding the cytokines and/or the co-stimulatory molecules for use in generating modified TILs for use in treating an individual with cancer or infection. In some embodiments, the kits comprise the compositions described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods treat an individual in need thereof and/or instructions for introducing cytokines and/or the co-stimulatory molecules into a TIL. The kits described herein may further include other materials, including other buffers, diluents, filters, needles, syringes, and package inserts with instructions for performing any methods described herein; e.g., instructions for treating an individual in need thereof or instructions for modifying TILs to contain intracellularly cytokines and/or the co-stimulatory molecules.

### Exemplary Embodiments

Embodiment 1. A method of modulating the activity and/or proliferative capacity of tumor-infiltrating lymphocytes (TILs), wherein the TILs are modified to increase expression of one or more co-stimulatory molecules and/or one or more cytokines.

Embodiment 2. A method of modulating the activity and/or proliferative capacity of tumor-infiltrating lymphocytes (TILs), wherein the TILs are modified to increase expression of one or more of co-stimulatory molecules.

Embodiment 3. The method of embodiment 1 or 2, wherein the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112.

Embodiment 4. The method of embodiment 1 or 2, wherein the co-stimulatory molecule is CD86.

Embodiment 5. A method of modulating the phenotype and/or proliferative capacity of TILs, wherein the TILs are modified to increase expression of one or more cytokines.

Embodiment 6. The method of any one of embodiments 1 and 3-5, wherein the TILs are modified to comprise a chimeric membrane-bound cytokine.

Embodiment 7. The method of embodiment 6, wherein the chimeric membrane-bound cytokine is a fusion protein comprising the cytokine and a transmembrane domain.

Embodiment 8. The method of embodiment 7, wherein the cytokine is joined to the transmembrane domain by a peptide linker.

Embodiment 9. The method of embodiment 8 wherein the peptide linker is (G4S) 3 (SEQ ID NO: 3) or (EAAAK) 3 (SEQ ID NO: 4).

Embodiment 10. The method of any one of embodiments 1 and 3-9, wherein the cytokine is a Type I cytokine.

Embodiment 11. The method of any one of embodiments 1 and 3-10, wherein the cytokine is IL-15, IL-12, IL-2, IFN α, IFN β, or IL-21 or functional variant thereof.

Embodiment 12. The method of embodiment 11, wherein the cytokine is IL-2 or a functional variant thereof and/or IL-12 or a functional variant thereof.

Embodiment 13. The method of any one of embodiments 7-12, wherein the chimeric membrane-bound cytokine comprises the amino acid sequence of SEQ ID NOs: 7-10.

Embodiment 14. The method of embodiment any one of embodiments 1-13, wherein the modified TILs comprise increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and
b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules.

Embodiment 15. The method of embodiment any one of embodiments 1-13, wherein the modified TILs comprises increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and
b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules.

Embodiment 16. The method of embodiment 14 or 15, wherein the method comprises:
(a) incubating the TILs with the nucleic acid encoding the chimeric membrane-bound cytokine before, during and/or after passing the cell suspension through the cell-deforming constriction
(b) incubating the TILs with the nucleic acid encoding the chimeric membrane-bound cytokine and the co-stimulatory molecule before, during and/or after passing the cell suspension through the cell-deforming constriction;
(c) incubating the TILs with the nucleic acid encoding the chimeric membrane-bound cytokine and the nucleic acid encoding the co-stimulatory molecule before, during and/or after passing the cell suspension through the cell-deforming constriction; or
(d) incubating the TILs with the nucleic acid encoding the chimeric membrane-bound cytokine and the nucleic acid encoding the one or more co-stimulatory molecules before, during and/or after passing the cell suspension through the cell-deforming constriction.

Embodiment 17. The method of embodiment 14 or 15, wherein the method comprises:
(a) incubating the TILs with the nucleic acid encoding the chimeric membrane-bound cytokine before passing the cell suspension through the cell-deforming constriction
(b) incubating the TILs with the nucleic acid encoding the chimeric membrane-bound cytokine and the co-stimulatory molecule before passing the cell suspension through the cell-deforming constriction;
(c) incubating the TILs with the nucleic acid encoding the chimeric membrane-bound cytokine and the nucleic acid encoding the co-stimulatory molecule before passing the cell suspension through the cell-deforming constriction; or
(d) incubating the TILs with the nucleic acid encoding the chimeric membrane-bound cytokine and the nucleic acid encoding the one or more co-stimulatory molecules before passing the cell suspension through the cell-deforming constriction.

Embodiment 18. The method of any one of embodiments 14-17, wherein one or more of the nucleic acids is mRNA.

Embodiment 19. The method of any one of embodiments 1-18, wherein the modified TILs have increased expression of one or more of T-bet, EOMES, TCF1, and CD62L compared to corresponding TILs that are not modified.

Embodiment 20. The method of any one of embodiments 1-18, wherein the modified TILs have increased expression of one or more of T-bet, EOMES, TCF1, and CD62L when cultured in the absence of exogenous cytokines, as compared to corresponding TILs that are not modified.

Embodiment 21. The method of embodiment 19 or 20, wherein the expression of one or more of T-bet, EOMES, TCF1, and CD62L in the modified TILs is increased by more than about 1.2-fold, 1.5-fold, 1.8-fold, 2-fold, 3-fold, 4-fold, 5-fold, 8-fold, or more than 10-fold as compared to corresponding TILs that are not modified.

Embodiment 21A. The method of any one of embodiments 1-21, wherein the modified TILs have increased expression of Granzyme B compared to corresponding TILs that are not modified.

Embodiment 21B. The method of any one of embodiments 1-21, wherein the modified TILs have increased expression of Granzyme B when cultured in the absence of exogenous cytokines, as compared to corresponding TILs that are not modified.

Embodiment 21C. The method of any one of embodiments 1-21, wherein the modified TILs have increased expression of Granzyme B when co-cultured with tumor cells in the absence of exogenous cytokines, as compared to corresponding TILs that are not modified.

Embodiment 21D. The method of embodiment 21A, 21B or 21C, wherein the expression of Granzyme B in the modified TILs is increased by about 1.2-fold, 1.5-fold, 1.8-fold, 2-fold, 3-fold, 4-fold, 5-fold, 8-fold, or 10-fold or more as compared to corresponding TILs that are not modified.

Embodiment 22. The method of any one of embodiments 1-21, wherein the modified TILs exhibit increased proliferation compared to corresponding TILs that are not modified.

Embodiment 23. The method of any one of embodiments 1-21, wherein the modified TILs exhibit increased proliferation when cultured in media not comprising exogenous cytokines, as compared to corresponding TILs that are not modified.

Embodiment 23A. The method of any one of embodiments 1-21, wherein the modified TILs exhibit increased proliferation when co-cultured with tumor cells in the absence of exogenous cytokines, as compared to corresponding TILs that are not modified.

Embodiment 24. The method of any one of embodiments 20-23A, wherein the exogenous cytokine is IL-2 and/or IL-12; optionally wherein the exogenous cytokine is IL-2.

Embodiment 25. A composition comprising modified TILs, wherein the TILs are modified to increase expression of one or more of co-stimulatory molecules and/or one or more cytokines.

Embodiment 26. A composition comprising modified TILs, wherein the TILs are modified to increase expression of one or more of co-stimulatory molecules.

Embodiment 27. The composition of embodiment 25 or 26, wherein the co-stimulatory molecule is B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112.

Embodiment 28. The composition of embodiment 27, wherein the co-stimulatory molecule is CD86.

Embodiment 29. A composition comprising modified TILs, wherein the TILs are modified to increase expression of one or more cytokines.

Embodiment 30. The composition of any one of embodiments 25 and 27-29, wherein the TILs are modified to comprise a chimeric membrane-bound cytokine.

Embodiment 31. The composition of embodiment 30, wherein the chimeric membrane-bound cytokine is a fusion protein comprising the cytokine and a transmembrane domain.

Embodiment 32. The composition of embodiment 31, wherein the cytokine is joined to the transmembrane domain by a peptide linker.

Embodiment 33. The composition of embodiment 32, wherein the peptide linker is (G4S) 3 (SEQ ID NO: 3) or (EAAAK) 3 (SEQ ID NO: 4).

Embodiment 34. The composition of any one of embodiments 25 and 27-33, wherein the cytokine is a Type I cytokine.

Embodiment 35. The composition of any one of embodiments 25 and 27-34, wherein the cytokine is IL-15, IL-12, IL-2, IFN α, IFN β, or IL-21 or functional variant thereof.

Embodiment 36. The method of embodiment 35, wherein the cytokine is IL-2 or a functional variant thereof and/or IL-12 or a functional variant thereof.

Embodiment 37. The composition of any one of embodiments 30-36, wherein the chimeric membrane-bound cytokine comprises the amino acid sequence of SEQ ID NOs: 7-10.

Embodiment 38. The composition of any one of embodiments 25-37, wherein the modified TILs comprise increased expression of one or more cytokines and/or one or more of co-stimulatory molecules, wherein the modified TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and
b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules.

Embodiment 39. The composition of any one of embodiments 25-37, wherein the TILs comprises increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and
b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules.

Embodiment 40. The composition of embodiment 38 or 39, wherein the process of preparing the modified TILs comprises:
(a) incubating the TILs with the nucleic acid encoding the chimeric membrane-bound cytokine before, during and/or after passing the cell suspension through the cell-deforming constriction
(b) incubating the TILs with the nucleic acid encoding the chimeric membrane-bound cytokine and the co-stimulatory molecule before, during and/or after passing the cell suspension through the cell-deforming constriction;
(c) incubating the TILs with the nucleic acid encoding the chimeric membrane-bound cytokine and the nucleic acid encoding the co-stimulatory molecule before, during and/or after passing the cell suspension through the cell-deforming constriction; or
(d) incubating the TILs with the nucleic acid encoding the chimeric membrane-bound cytokine and the nucleic acid encoding the one or more co-stimulatory molecules before, during and/or after passing the cell suspension through the cell-deforming constriction.

Embodiment 41. The composition of any one of embodiment 38-39, wherein the process of preparing the modified TILs comprises:
(a) incubating the TILs with the nucleic acid encoding the chimeric membrane-bound cytokine before passing the cell suspension through the cell-deforming constriction
(b) incubating the TILs with the nucleic acid encoding the chimeric membrane-bound cytokine and the co-stimulatory molecule before passing the cell suspension through the cell-deforming constriction;
(c) incubating the TILs with the nucleic acid encoding the chimeric membrane-bound cytokine and the nucleic acid encoding the co-stimulatory molecule before passing the cell suspension through the cell-deforming constriction; or
(d) incubating the TILs with the nucleic acid encoding the chimeric membrane-bound cytokine and the nucleic acid encoding the one or more co-stimulatory molecules before passing the cell suspension through the cell-deforming constriction.

Embodiment 42. The composition of any one of embodiments 38-41, wherein one or more of the nucleic acids is mRNA.

Embodiment 43. The composition of any one of embodiments 25-42, wherein the modified TILs have increased expression of one or more of T-bet, EOMES, TCF1, and CD62L compared to corresponding TILs that are not modified.

Embodiment 44. The composition of any one of embodiments 25-43, wherein the modified TILs have increased expression of one or more of T-bet, EOMES, TCF1, and CD62L when cultured in the absence of exogenous cytokines, compared to corresponding TILs that are not modified.

Embodiment 45. The composition of embodiment 43 or 44, wherein the expression of one or more of T-bet, EOMES, TCF1, and CD62L in modified TILs is increased by more than about 1.2-fold, 1.5-fold, 1.8-fold, 2-fold, 3-fold, 4-fold, 5-fold, 8-fold, or more than 10-fold compared to the corresponding TILs that are not modified.

Embodiment 45A. The composition of any one of embodiments 25-45, wherein the modified TILs have increased expression of Granzyme B compared to corresponding TILs that are not modified.

Embodiment 45B. The composition of any one of embodiments 25-45, wherein the modified TILs have increased expression of Granzyme B when cultured in the absence of exogenous cytokines, as compared to corresponding TILs that are not modified.

Embodiment 45C. The composition of any one of embodiments 25-45, wherein the modified TILs have increased expression of Granzyme B when co-cultured with tumor cells in the absence of exogenous cytokines, as compared to corresponding TILs that are not modified.

Embodiment 45D. The composition of embodiment 45A or 45B or 45C, wherein the expression of Granzyme B in the modified TILs is increased by about 1.2-fold, 1.5-fold, 1.8-fold, 2-fold, 3-fold, 4-fold, 5-fold, 8-fold, or 10-fold or more as compared to corresponding TILs that are not modified.

Embodiment 46. The composition of any one of embodiments 25-45, wherein the modified TILs exhibit increased proliferation compared to corresponding TILs that are not modified.

Embodiment 47. The composition of any one of embodiments 25-45, wherein the modified TILs exhibit increased proliferation when cultured in media not comprising exogenous cytokines, as compared to corresponding TILs that are not modified.

Embodiment 47A. The composition of any one of embodiments 25-45, wherein the modified TILs exhibit increased proliferation when co-cultured with tumor cells in the absence of exogenous cytokines, as compared to corresponding TILs that are not modified.

Embodiment 48. The composition of any one of embodiments 44-47A, wherein the exogenous cytokine is IL-2 and/or IL-12; optionally wherein the exogenous cytokine is IL-2.

Embodiment 49. A method of modulating an immune response in an individual, comprising administering the modified TILs to an individual, wherein the modified TILs are prepared according to the method of any one of embodiments 1-24.

Embodiment 50. A method of modulating an immune response in an individual, comprising administering the composition of any one of embodiments 25-48.

Embodiment 51. A method for treating a cancer, an infectious disease, or a viral-associated disease in an individual, comprising administering the modified TILs to an individual, wherein the modified TILs are prepared according to the method of any one of embodiments 1-24.

Embodiment 52. A method for treating a cancer, an infectious disease, or a viral-associated disease in an individual, comprising administering the composition of any one of embodiments 25-48.

Embodiment 53. The method of any one of embodiments 49-52, wherein the method comprises multiple administration of the modified TILs, or multiple administration of the composition.

Embodiment 54. The method of any one of embodiments 49-53, wherein the modified TILs is administered intravenously or intratumourally.

Embodiment 55. The method of any one of embodiments 49-54, wherein the individual is a human.

Embodiment 56. The method of any one of embodiments 49-55, wherein the modified TILs are administered prior to, concurrently with, or following administration of another therapy.

Embodiment 57. A pharmaceutical composition for stimulating an immune response in an individual, wherein the composition comprises an effective amount of composition of any one of embodiments 25-48.

Embodiment 58. A pharmaceutical composition for use as a medicine, wherein the pharmaceutical composition comprises an effective amount of composition of any one of embodiments 25-48.

Embodiment 59. A pharmaceutical composition for treating a cancer, an infectious disease, or a viral-associated disease in an individual, wherein the pharmaceutical composition comprises an effective amount of composition of any one of embodiments 25-48.

Embodiment 60. The pharmaceutical composition of embodiment 58 or 59, wherein the composition comprising modified TILs is administered prior to, concurrently with, or following administration of another therapy.

Embodiment 61. The method of any one of embodiments 1-24 and 49-56 or the composition of any one of embodiments 25-48 and 57-60, wherein the width of the constriction is about 10% to about 99% of the mean diameter of the input TILs.

Embodiment 62. The method of any one of embodiments 1-24 and 49-56 or the composition of any one of embodiments 25-48, wherein the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm.

Embodiment 63. The method of any one of embodiments 1-24 and 49-56 or the composition of any one of embodiments 25-48 and 57-60, wherein the width of the constriction is about 3 μm to about 5 μm.

Embodiment 64. The method of any one of embodiments 1-24 and 49-56 or the composition of any one of embodiments 25-48 and 57-60, wherein the width of the constriction is about 4 μm.

Embodiment 65. The method of any one of embodiments 1-24 and 49-56 or the composition of any one of embodiments 25-48 and 57-60, wherein the cell suspension comprising the plurality of input TILs are passed through multiple constrictions wherein the multiple constrictions are arranged in series and/or in parallel.

Embodiment 66. A kit for use in the method of any one of embodiments 1-24, 49-56 and 61-65.

Embodiment 67. A kit comprising the composition of any one of embodiments 25-48 and 57-65.

Embodiment 68. The kit of embodiment 66 or 67, wherein the kit further comprises one or more of buffers, diluents, filters, needles, syringes, or package inserts with instructions for administering the composition to an individual.

Embodiment 69. A method of producing TILs comprising a chimeric membrane-bound cytokine, the method comprising introducing a nucleic acid encoding the chimeric membrane-bound cytokine to the TILs.

Embodiment 70. The method of embodiment 69, wherein the TILs comprising the chimeric membrane-bound cytokine are prepared by:
a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and
b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed; thereby generating TILs comprising a chimeric membrane-bound cytokine.

Embodiment 71. The method of embodiment 70, wherein the method comprises incubating the TILs with the nucleic acid encoding the chimeric membrane-bound cytokine thereof before, during and/or after passing the cell suspension through the cell-deforming constriction.

Embodiment 72. The method of embodiment 70, wherein the method comprises incubating the TILs with the nucleic acid encoding the chimeric membrane-bound cytokine before passing the cell suspension through the cell-deforming constriction.

Embodiment 73. The method of any one of embodiments 69-72, wherein the nucleic acid encoding the chimeric membrane-bound cytokine is an mRNA encoding the chimeric membrane-bound cytokine.

Embodiment 74. The method of any one of embodiments 70-73, wherein the width of the constriction is about 10% to about 99% of the mean diameter of the input TILs.

Embodiment 75. The method of any one of embodiments 70-74, wherein the width of the constriction is about 5 μm to about 12 μm, or about 6 μm to about 12 μm, or about 8 μm to about 11 μm, or about 9 μm to about 11 μm, or about 12 μm to about 15 μm.

Embodiment 76. The method of any one of embodiments 70-75, wherein the width of the constriction is about 10 μm.

Embodiment 77. The method of any one of embodiments 70-76, wherein the width of the constriction is about 8 μm.

Embodiment 78. The method of any one of embodiments 70-77, wherein the cell suspension comprising the plurality of input TILs are passed through multiple constrictions wherein the multiple constrictions are arranged in series and/or in parallel.

Embodiment 79. Use of a pharmaceutical composition in the manufacture of a medicament for stimulating an immune response in an individual, wherein the pharmaceutical composition comprises an effective amount of composition of any one of embodiments 25-48.

Embodiment 80. Use of a pharmaceutical composition in the manufacture of a medicament for treating a cancer, an infectious disease, or a viral-associated disease in an individual, wherein the pharmaceutical composition comprises an effective amount of composition of any one of embodiments 25-48.

Embodiment 81. The use of embodiment 79 or 80, wherein the pharmaceutical composition is formulated for multiple administration.

Embodiment 82. The use of any one of embodiments 79-81, wherein the pharmaceutical composition is administered intravenously or intratumourally.

Embodiment 83. The use of any one of embodiments 79-82, wherein the individual is a human.

Embodiment 84. The use of any one of embodiments 79-83, wherein the pharmaceutical composition is formulated for administration prior to, concurrently with, or following administration of another therapy

### Examples

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1: Tumor Infiltrating Lymphocytes (TILs) Express Membrane Bound Cytokines Over 48 Hours Subsequent to Constriction Mediated Delivery mRNAs Encoding Membrane-Bound Cytokines

This example shows the expression of membrane-bound IL-2 (e.g., mbIL-2) and/or membrane-bound IL-12 (e.g., mbIL-12) in tumor infiltrating lymphocytes (TILs) over 48 hours, subsequent to constriction mediated delivery of mbIL-2 and/or mbIL-12 mRNA.

### Methods

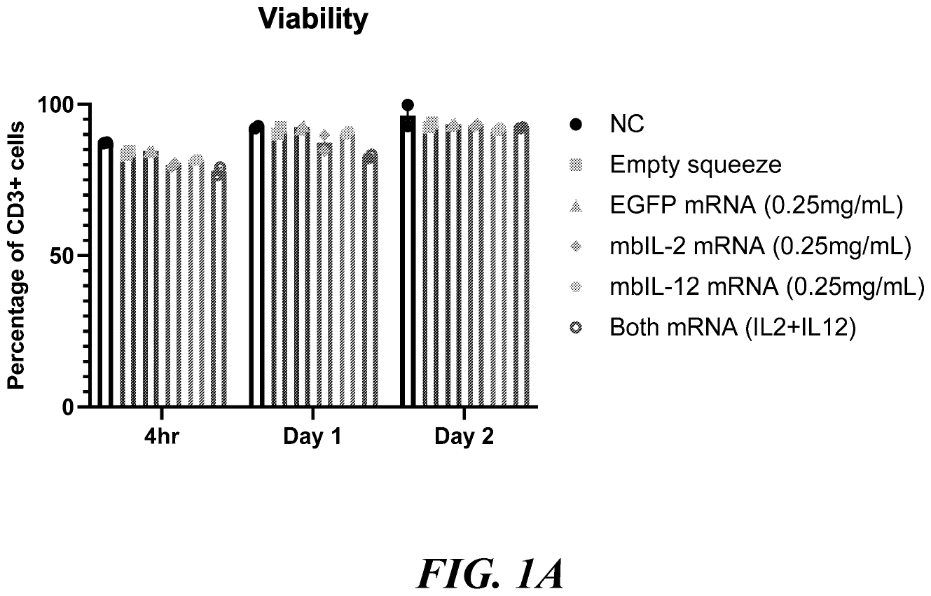

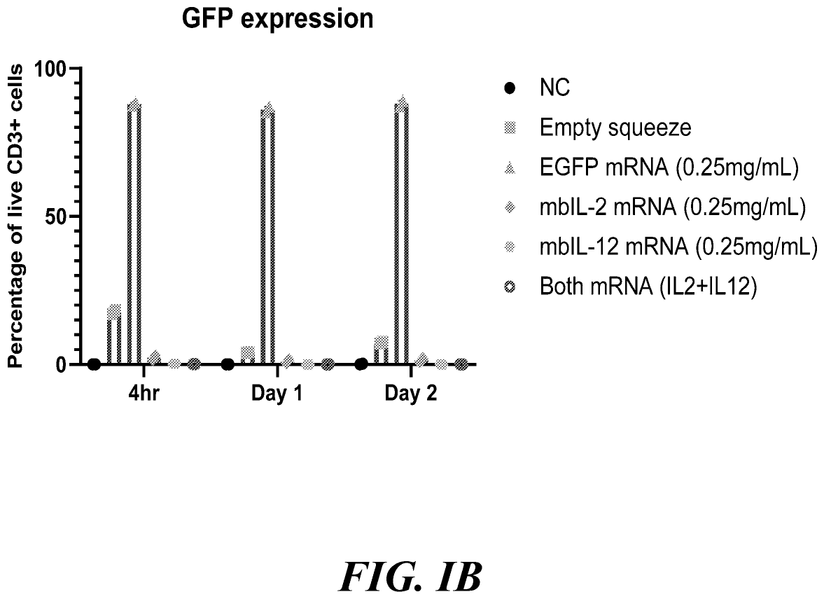

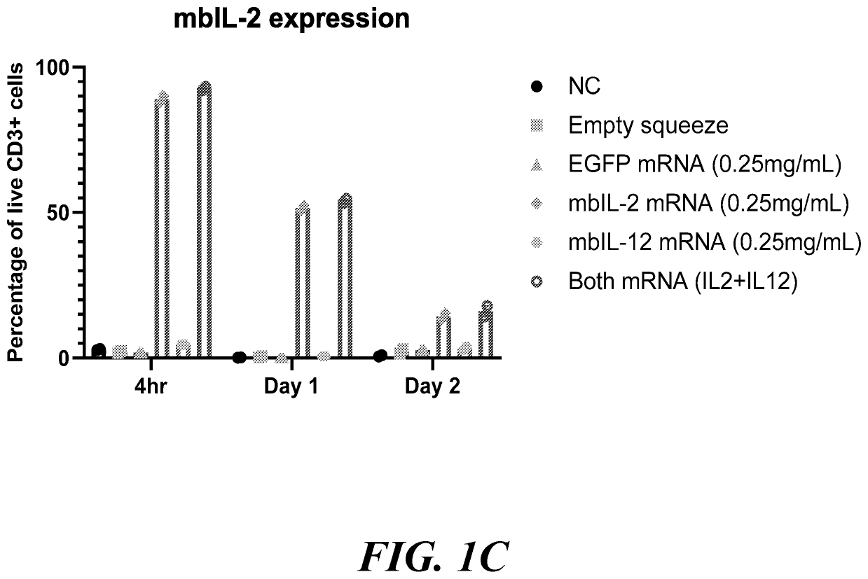

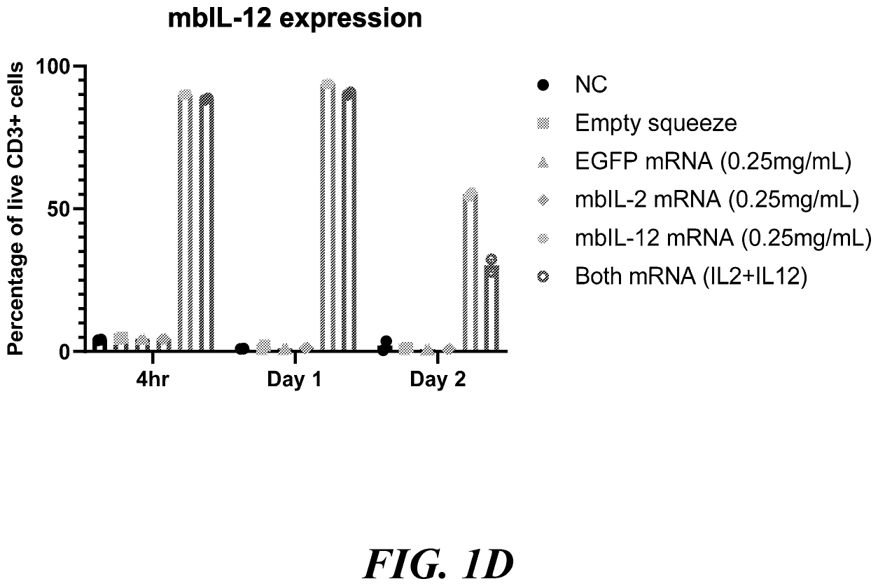

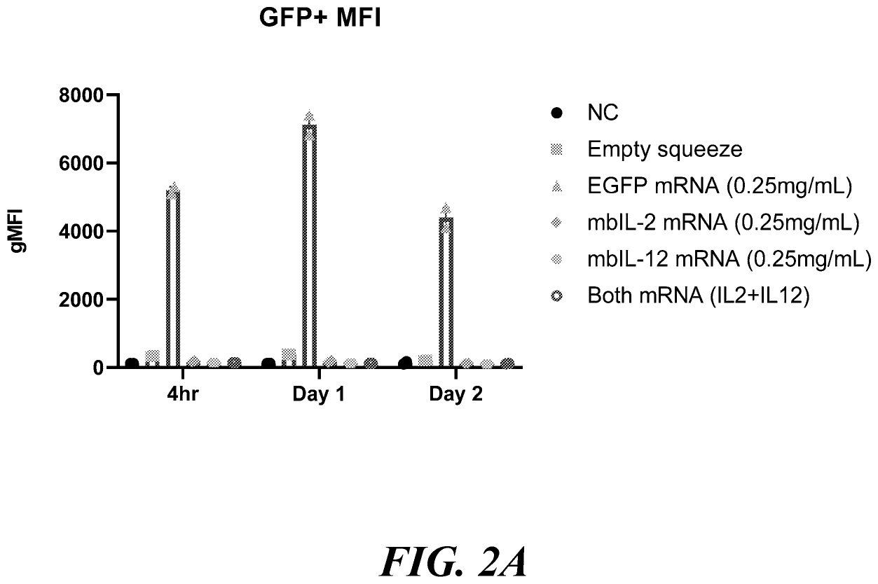

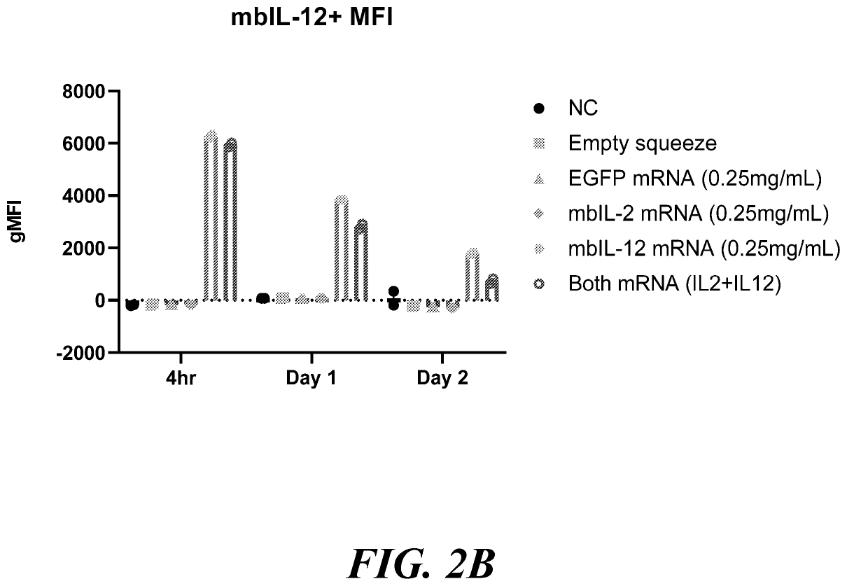

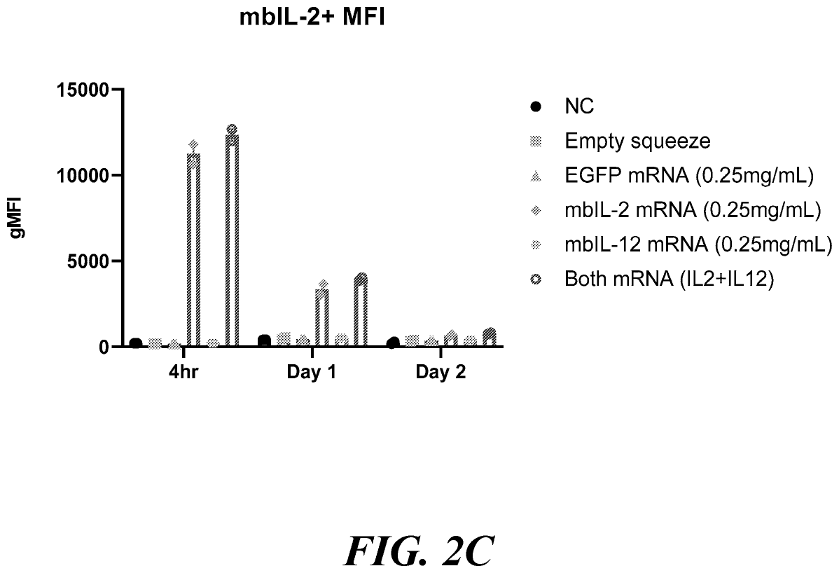

TILs were thawed and cultured in media comprising a high concentration of IL-2 (3000 IU/mL) for 24 hours. Following culture, mRNA encoding mbIL-2 (0.25 mg/mL), mRNA encoding mbIL-12 (0.25 mg/mL), or mRNAs encoding mbIL-2 and mbIL-12 (0.25 mg/mL each; total 0.5 mg/mL) were delivered to the TILs by constriction mediated delivery (“squeezing”). Specifically, TILs were squeeze-processed at 30 psi using a ST-10-040-70 chip (10 μm length, 4 μm width, 70 μm depth) in the presence of the mRNAs as described. Constriction mediated delivery was likewise performed with empty payload and GFP mRNA to serve as controls for downstream analysis. The TILs comprising the squeeze-delivered mRNAs were resuspended in culture media comprising a high concentration of IL-2 (3000 IU/mL). Fluorescence-activated cell sorting (FACS) was used to determine the expression of the membrane bound cytokines at 4 hours, 24 hours, and 48 hours following resuspension. In particular, cellular viability, percentage of cells expressing GFP mbIL-2 and/or mbIL-12, as well as mean fluorescence intensities (e.g., MFI) of GFP expression, mbIL-2 expression, and mbIL-12 expression, were evaluated (FIGS. 1A-1D and FIGS. 2A-2C). Table 1 provides an outline of the FACS protocols used to evaluate the TILs.

TABLE 1
Flow chart corresponding to the assays from Example 1.

| | Emission | | | | Antibody | Final |
|---|---|---|---|---|---|---|
| | (nm) | Channel | Fluorochromes | Target | Clone | Dilution |
| Violet | 440/50 | VL-1 | Pacific Blue | CD3 | SK7 | 1:200 |
| (405 nm) | | | | | | |
| Blue | 530/30 | BL-1 | GFP | mRna | N/A | Delivery |
| (488 nm) | | | | Expression | | at 0.25 |
| | | | | | | mg/mL |
| Yellow | 585/16 | YL-1 | PE | mbIL12 | 20C2 | 1:200 |
| (561 nm) | | | | | | |
| Red | 670/14 | RL-1 | APC | mbIL2 | MQ1- | 1:200 |
| (638 nm) | | | | | 17H12 | |
| | 780/60 | RL-3 | Live/Dead | Viability | | 1:1000 |
| | | | near IR | | | |

### Results

As shown in FIG. 1A, viability was consistently between 80-90% at all measured time points post-squeezing (4 hours, Day 1, and Day 2). The viability of TILs increases over time in high IL-2 culture media, as shown in the comparison of viability of TILs at 4 hours versus Day 2.

The GFP mRNA positive control indicated that mRNAs were effectively delivered and expressed in TILs using the constricted mediated delivery method (FIG. 1B and FIG. 2A). TILs successfully expressed mbIL-2 and mbIL-12, and both mbIL-2 and mbIL-12 simultaneously, when mRNA was delivered via a constriction mediated delivery method as indicated by both percentage of cells expression the cytokine, and MFI expression (see, e.g., FIGS. 1C-1D and FIGS. 2B-2C).

### Example 2: TILs Express Membrane Bound Cytokines Over 7 Days when Cultured with Varying IL-2 Concentrations Upon Constriction Mediated Delivery of mRNAs Encoding Membrane Bound Cytokines

This example shows the expression of membrane-bound IL-2 (e.g., mbIL-2) or membrane-bound IL-12 (e.g., mbIL-12) over 7 days when culture in the presence of varying IL-2 concentrations following constriction mediated delivery of mRNAs encoding mbIL-2 and mbIL-12 mRNA.

### Methods

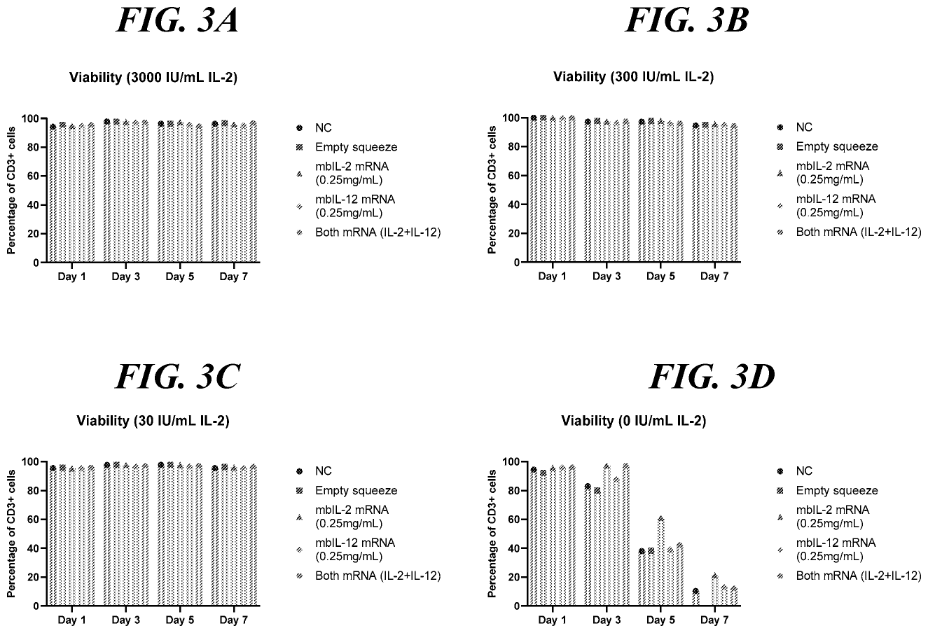

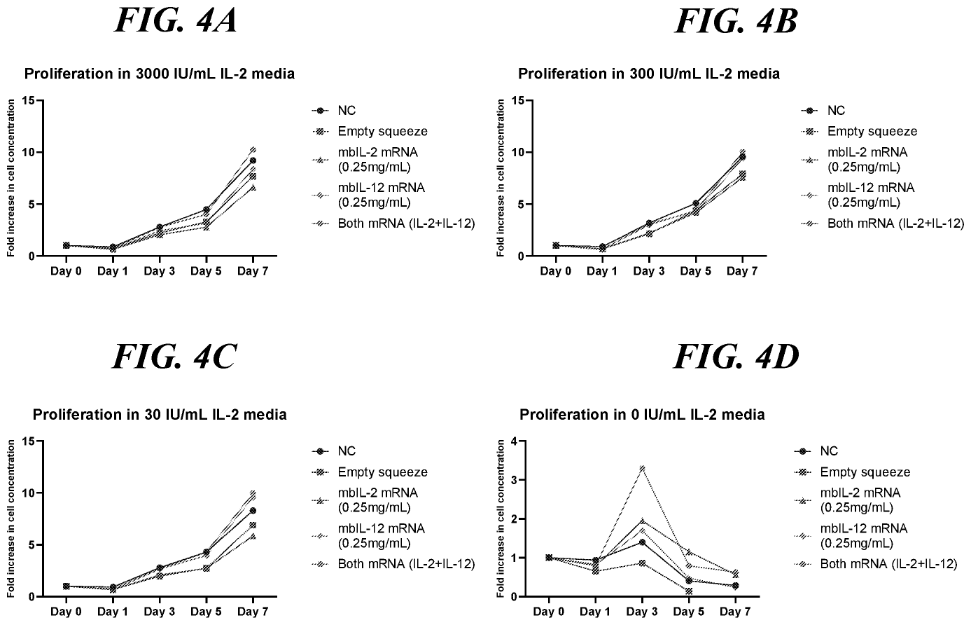

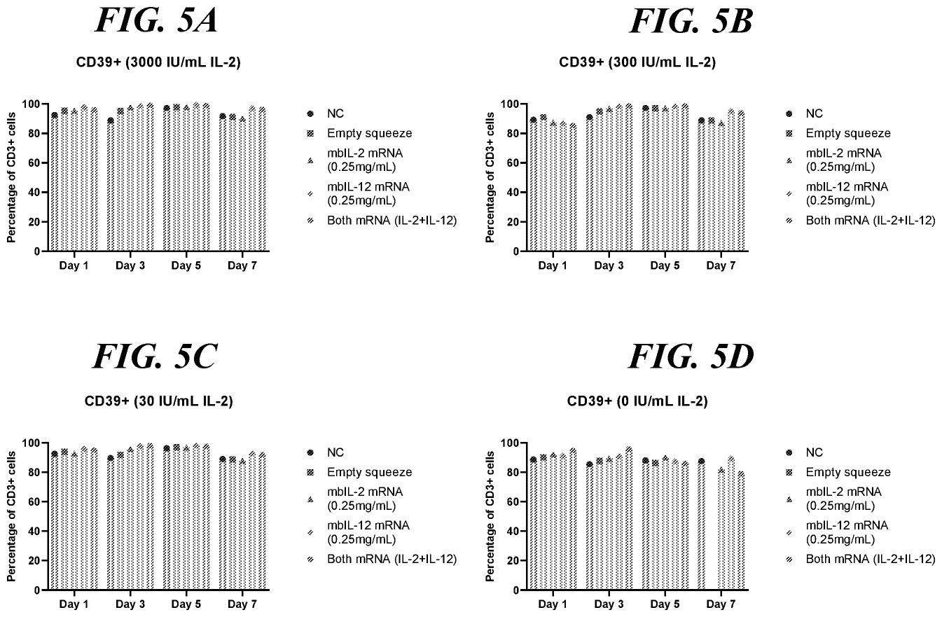

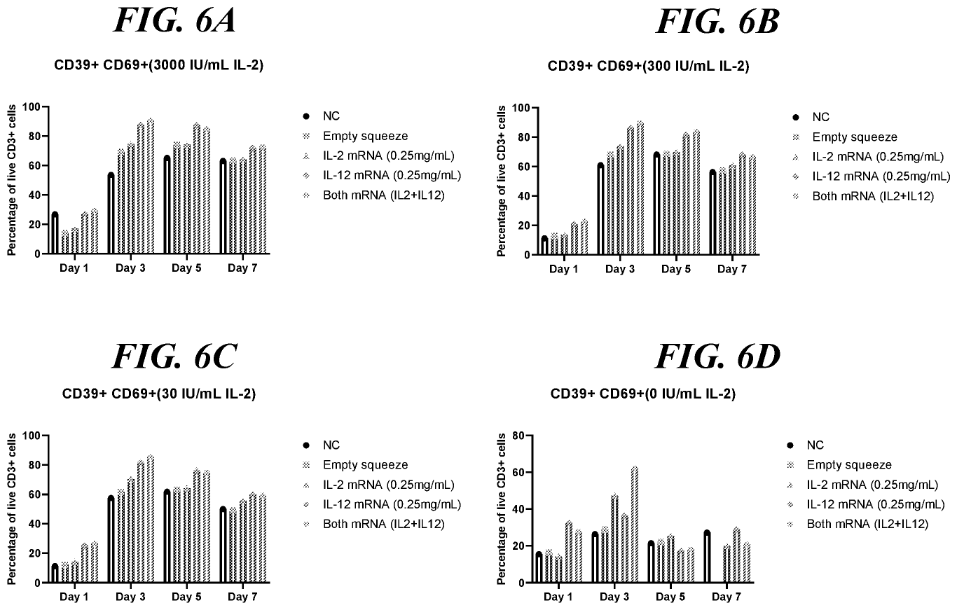

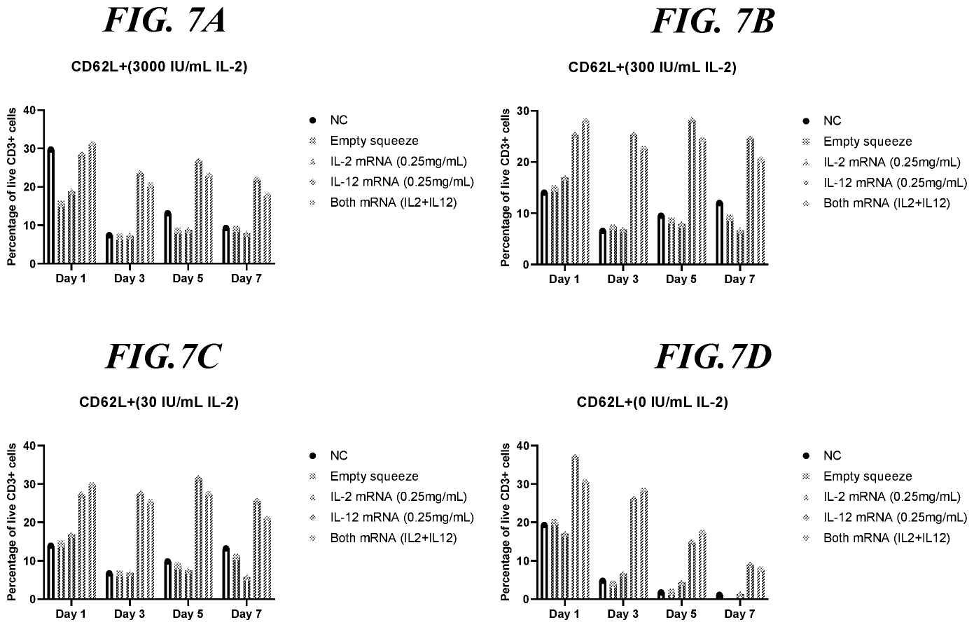

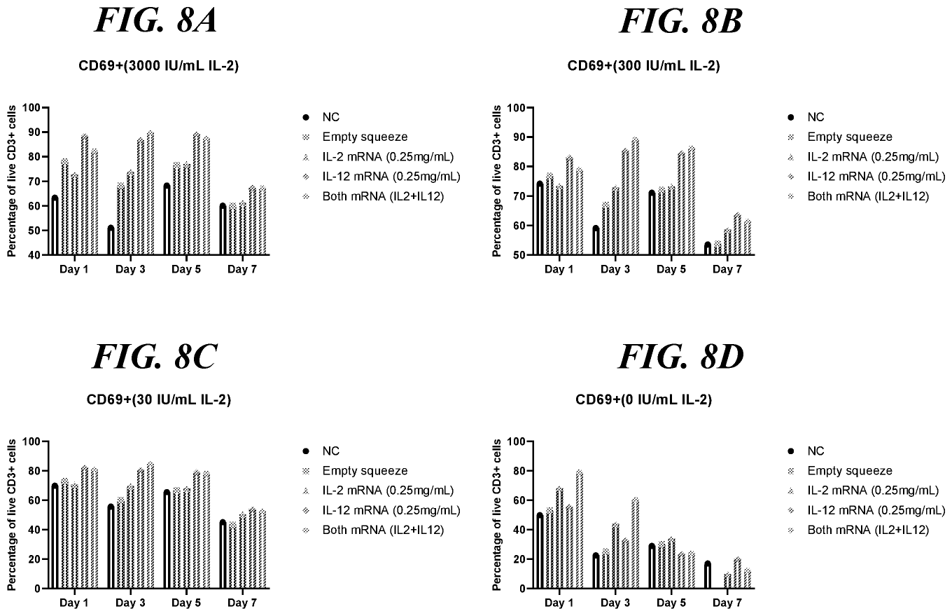

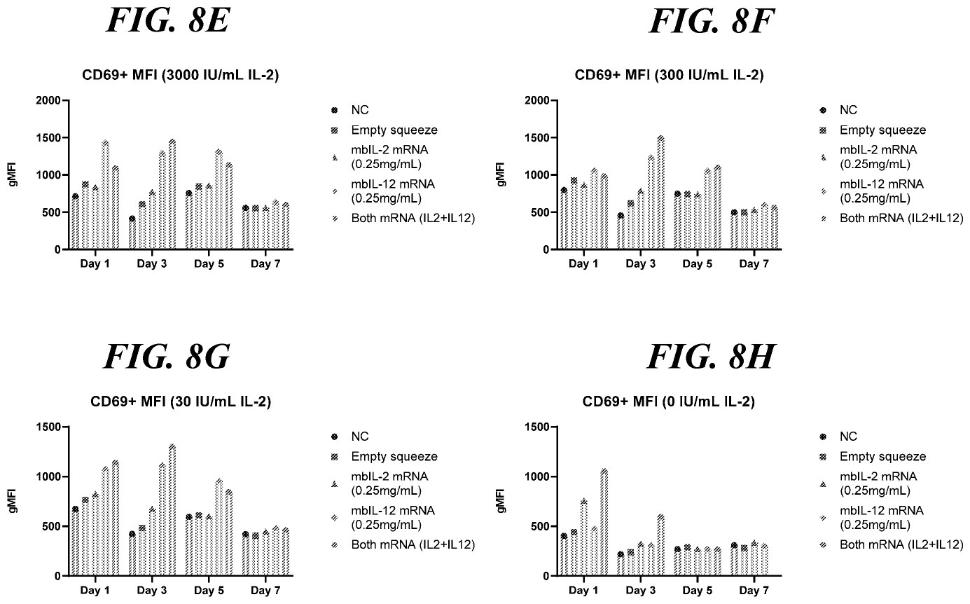

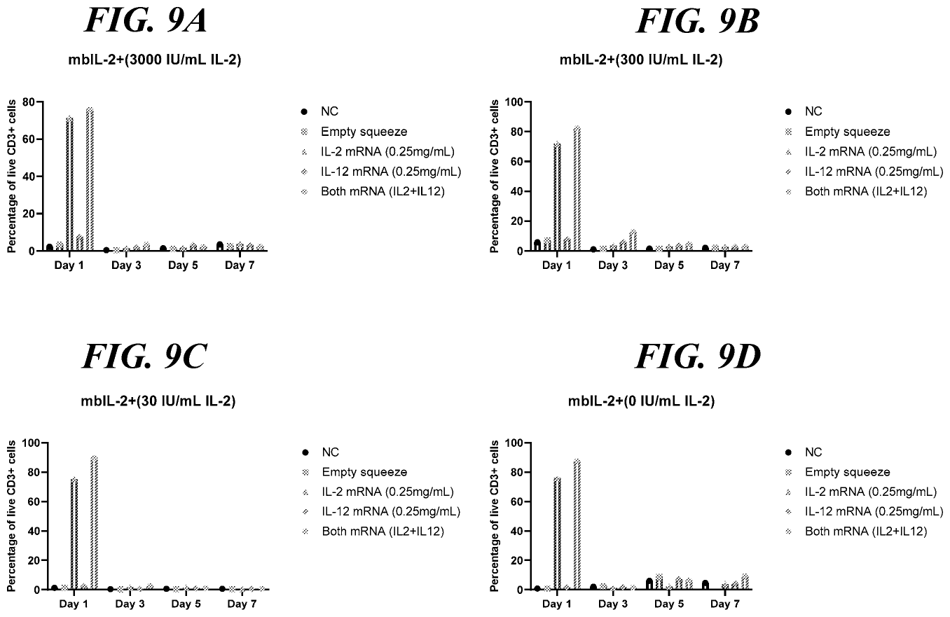

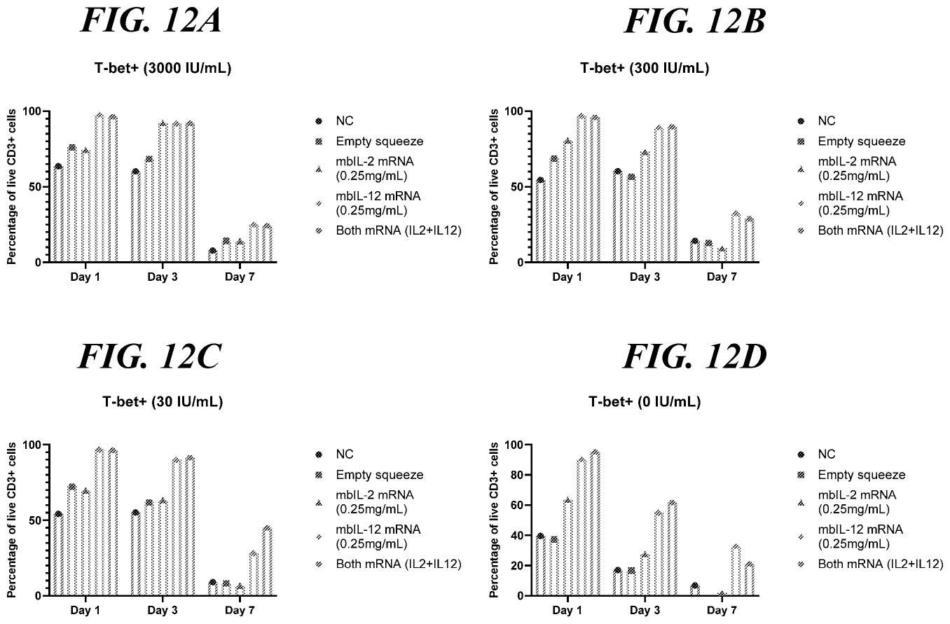

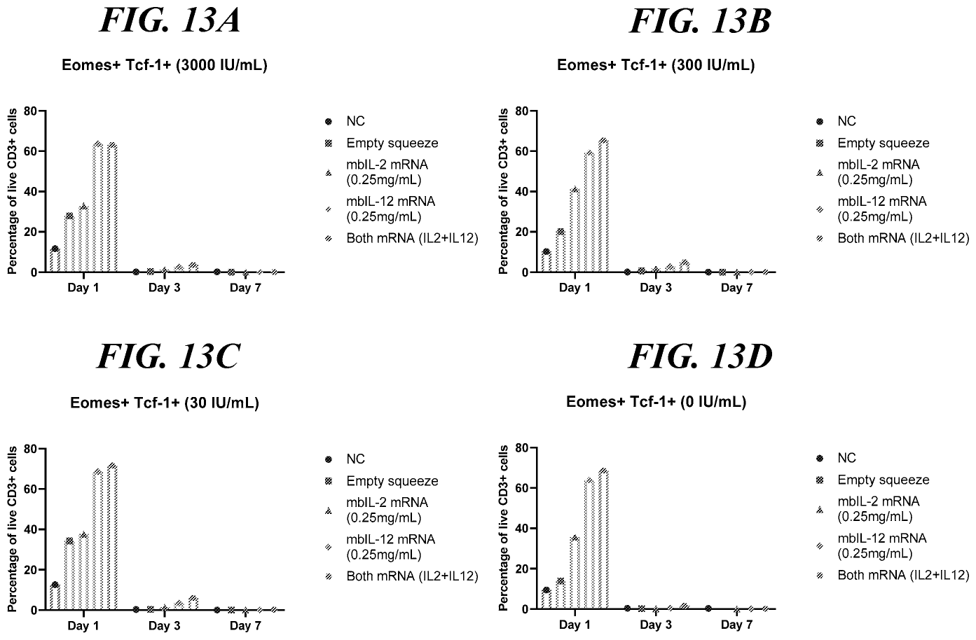

TILs were thawed and cultured in media comprising a high concentration of IL-2 (3000 IU/mL) for 24 hours. Following culture, mRNA encoding mbIL-2 (0.25 mg/mL), mbIL-12 (0.25 mg/mL), or both mbIL-2 and mbIL-12 (0.25 mg/mL) were delivered to the TILs by constriction mediated delivery. TILs squeeze-processed at 30 psi using a ST-10-040-70 chip (10 μm length, 4 μm width, 70 μm depth) in the presence of the mRNAs as described. The TILs comprising the squeeze-delivered mRNAs were resuspended in culture media comprising the following concentrations of IL-2 for 7 total days: 3000 IU/mL, 300 IU/mL, 30 IU/mL, and 0 IU/mL. Fluorescence-activated cell sorting (FACS) was used to determine the expression of cytokines at 1 day, 3 days, and 7 days following resuspension. In particular, cellular viability, proliferation, percentage of cells expressing cytokines, and the mean fluorescence intensities (e.g., MFI) of the expression of mbIL-2 and mbIL-12 were evaluated. Viability of TILs following squeeze-delivery of mRNAs and incubation in the presence of IL-2 is shown in FIGS. 3A-3D. Proliferation of TILs Viability of TILs following squeeze-delivery of mRNAs and incubation in the presence of IL-2 is shown in FIGS. 4A-4D. Expression of CD39 on TILs following squeeze-delivery of mRNAs and incubation in the presence of IL-2 is shown in FIGS. 5A-5D. Co-expression of CD39 and CD69 in TILs following squeeze-delivery of mRNAs and incubation in the presence of IL-2 is shown in FIGS. 6A-6D. Expression of CD62L on TILs following squeeze-delivery of mRNAs and incubation in the presence of IL-2 is shown in FIGS. 7A-7D. Expression of CD69 on TILs following squeeze-delivery of mRNAs and incubation in the presence of IL-2 is shown in FIGS. 8A-8H. FIGS. 9A-9D, 10A-10D and 11A-11D show the percentage of TILs expressing mbIL-2, or mbIL-12, or both mbIL-2 and mbIL-12, respectively. The percentage of TILs expressing T-bet following squeeze-delivery of mRNAs and incubation in the presence of IL-2 is shown in FIGS. 12A-12D. The percentage of TILs expressing Eomes and TCF-1 following squeeze-delivery of mRNAs and incubation in the presence of IL-2 is shown in FIGS. 13A-13D. Each assay was performed for each concentration of IL-2 in the culture media.

Tables 2 and 3 provide an outline of the FACS protocols used to evaluate the surface and nuclear components of the TILs.

TABLE 2
Flow chart corresponding to the cell surface stain assays from Example 2.

| | Emission | | | | Final |
|---|---|---|---|---|---|
| | (nm) | Channel | Fluorochromes | Target | Dilution |
| Violet | 440/50 | VL-1 | Pacific Blue | IL-12 | 1:200 |
| (405 nm) | 603/28 | VL-3 | BV605 | CD62L | 1:200 |
| Blue | 530/30 | BL-1 | FITC | CD39 | 1:200 |
| (488 nm) | | | | | |
| Yellow | 585/16 | YL-1 | PE | CD3 | 1:200 |
| (561 nm) | 780/60 | YL-4 | PE-Cy7 | CD69 | 1:200 |
| Red | 670/14 | RL-1 | APC | IL-2 | 1:200 |
| (638 nm) | 780/60 | RL-3 | NIR | Live/Dead | 1:1000 |

TABLE 3
Flow chart corresponding to the nuclear stain assays from Example 2.

| | Emission | | | | Final |
|---|---|---|---|---|---|
| | (nm) | Channel | Fluorochromes | Target | Dilution |
| Violet | 440/50 | VL-1 | BV421 | T-bet | 1:200 |
| (405 nm) | | | | | |
| Blue | 530/30 | BL-1 | FITC | Eomes | 1:200 |
| (488 nm) | 695/40 | BL-3 | PerCP/Cy5.5 | CD3 | 1:200 |
| Yellow | 585/16 | YL-1 | PE | TCF-1 | 1:200 |
| (561 nm) | | | | | |
| Red | 780/60 | RL-3 | NIR | Live/Dead | 1:1000 |
| (638 nm) | | | | | |

### Results

As shown in FIGS. 3A-3C, viability was consistently greater than about 85% for all cells tested in 3000 IU/mL, 300 IU/mL, and 30 IU/mL IL-2 culture media for all time points. The viability of TILs in media lacking IL-2 was greater than about 85% until day 3 (FIG. 3D). Proliferation increased for all TILs in media with 3000 IU/mL, 300 IU/mL, and 30 IU/mL IL-2 (FIGS. 4A-4C), and proliferation of TILs cultured in media lacking IL-2 was similar to that of corresponding TILs cultured in 3000 IU/mL IL-2 media up to day 3.

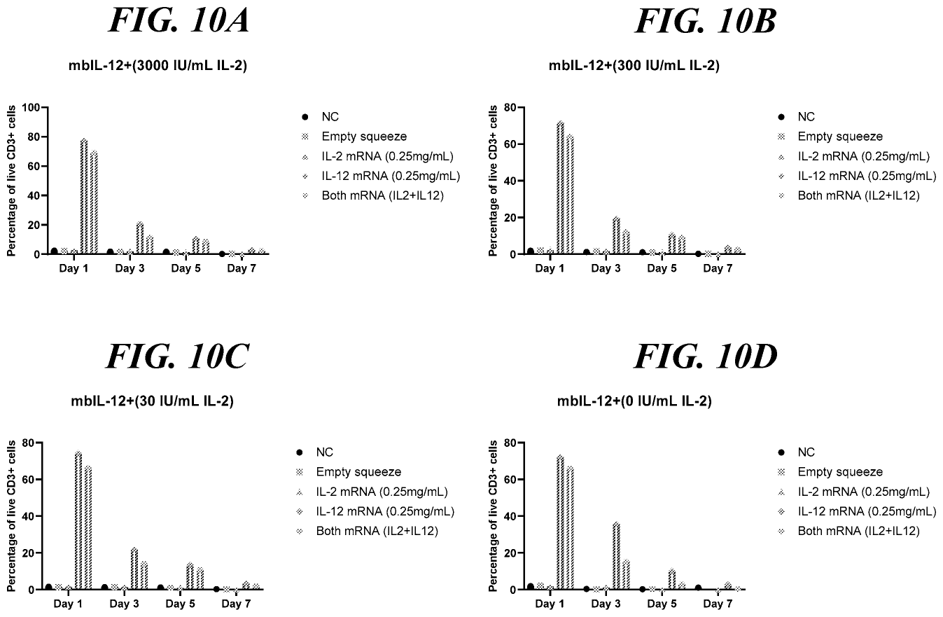

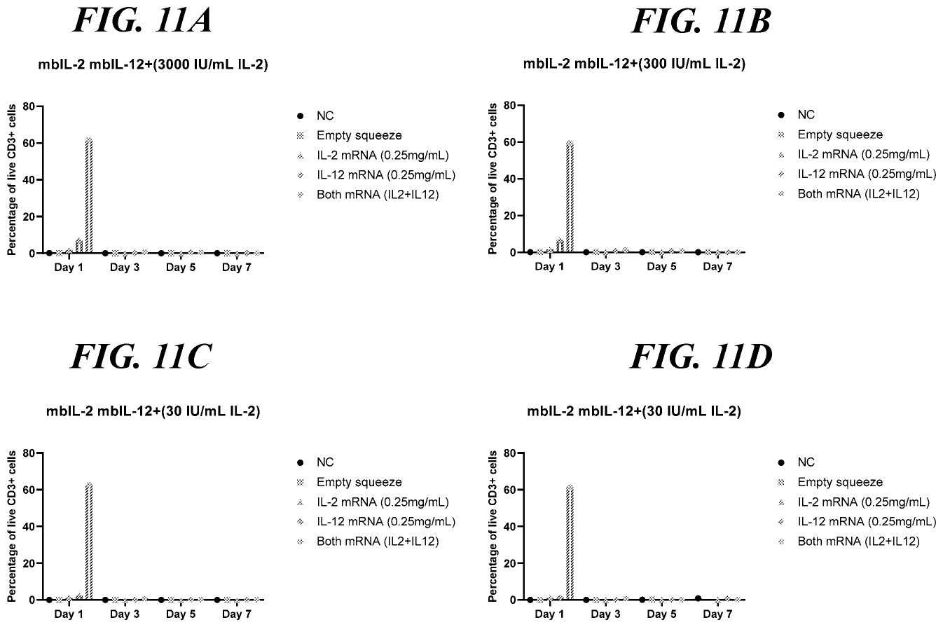

TILs successfully expressed mbIL-2, mbIL-12, or both mbIL-2 and mbIL-12 simultaneously upon squeeze mediated deliver of mRNA as indicated by percentage of cells expressing the respective cytokines (see, e.g., FIGS. 9A-9D, 10A-10D, and 11A-11D). As shown in FIGS. 9A-9D, expression of mbIL-2 was reduced over 2 days after squeeze delivery under each IL-2 media concentration. The mbIL-2 expression was highest on Day 1 following mRNA delivery, and negligible by Day 3 (FIGS. 9A-9D). Expression of mbIL-12 was similarly reduced over 2 days after squeeze-delivery at each IL-2 media concentration during incubation following squeeze-delivery of mRNAs (FIGS. 10A-10D). As shown in FIGS. 10A-10D, mbIL-12 expression was highest on Day 1 following squeeze delivery of mRNA and gradually decreased over time. Similarly, co-expression of mb-IL2 and mbIL-12 was reduced over 2 days after squeeze-delivery at each IL-2 media concentration during incubation, following squeeze-delivery of the respective mRNAs (FIG. 11A-11D).

The phenotypes of TILs following squeeze delivery of mRNAs followed by incubation with IL-2 was also evaluated. FIGS. 5A-5D illustrate that expression of CD39 was similar across all TILs, regardless of IL-2 culture conditions. Expression of CD62L (central memory T-cells) was highest on Day 1 upon delivery of a combination of mbIL-2 and mbIL-12 mRNA delivery followed by incubation with IL-2 in the culture media (FIGS. 7A-7D). Expression of CD69 was similar on Day 1 and Day 7 in all TILs cultured in IL-2 media, but decreased by Day 3 (FIGS. 8A-8H). As shown in FIGS. 12A-12D, T-bet expression was enhanced with greater concentrations of IL-2 in the culture media in TILs expressing mbIL-2, mbIL-12, or both mbIL-2 and mbIL-12. TILs showed higher expression of Eomes and TCF-1 at Day 3 following squeeze mediated mRNA delivery (FIGS. 13A-13D).

### Example 3: TILs Express Membrane Bound Cytokines Over 3 Days when Cultured with Varying IL-2 Concentrations Upon Constriction Mediated Delivery of mRNAs Encoding mbIL-2, mbIL-12, and CD86

This example shows expression of mbIL-2 and mbIL-12 by TILs upon squeeze-mediated deliver of mRNA encoding mbIL-2 and mbIL-12 or CD86 and their impact on TIL phenotypes.

### Methods

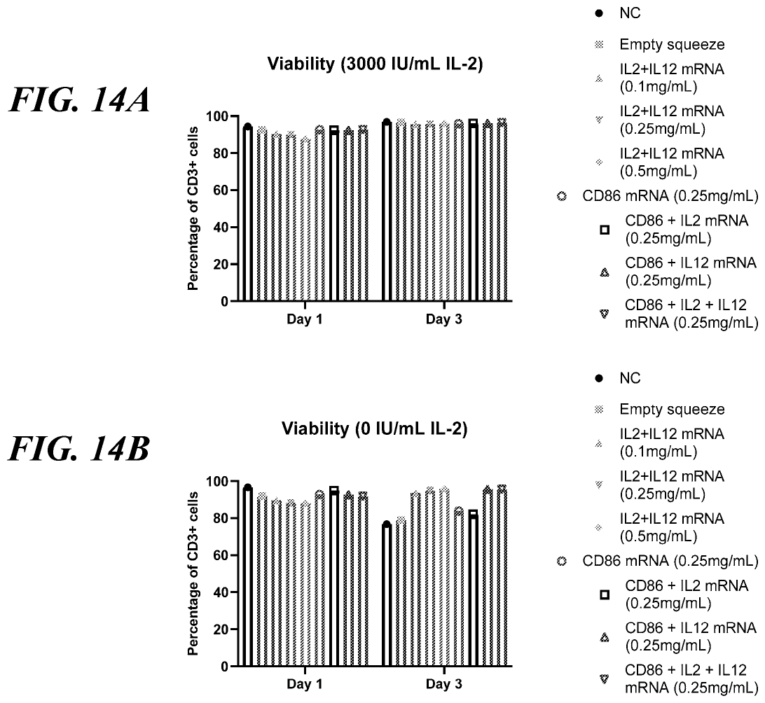

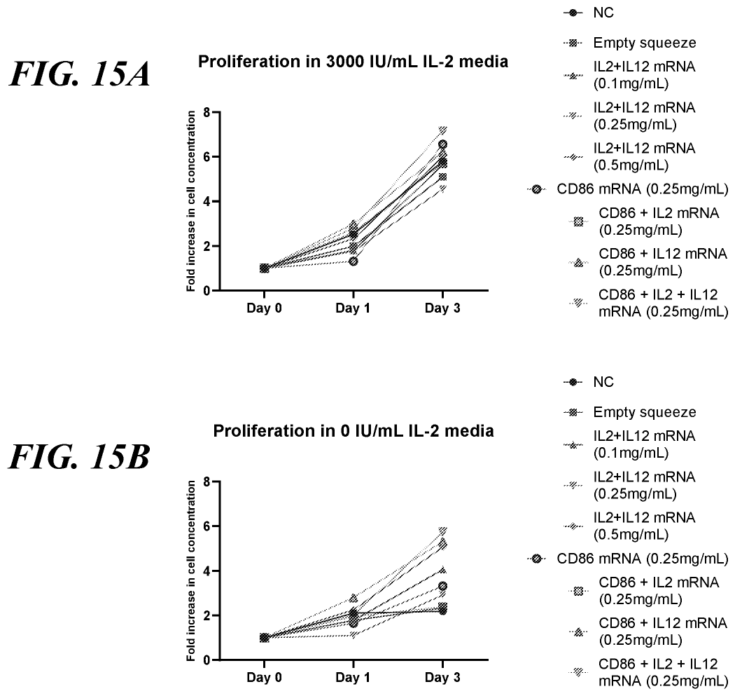

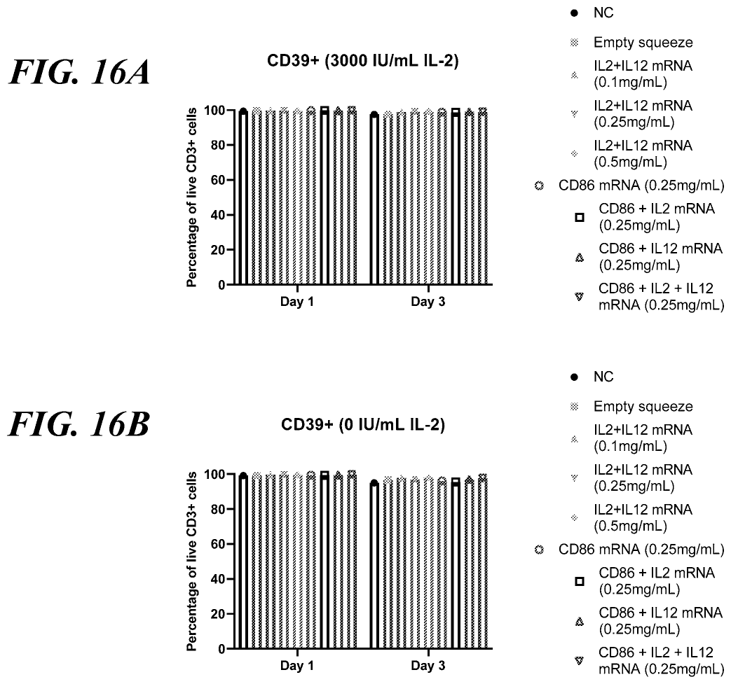

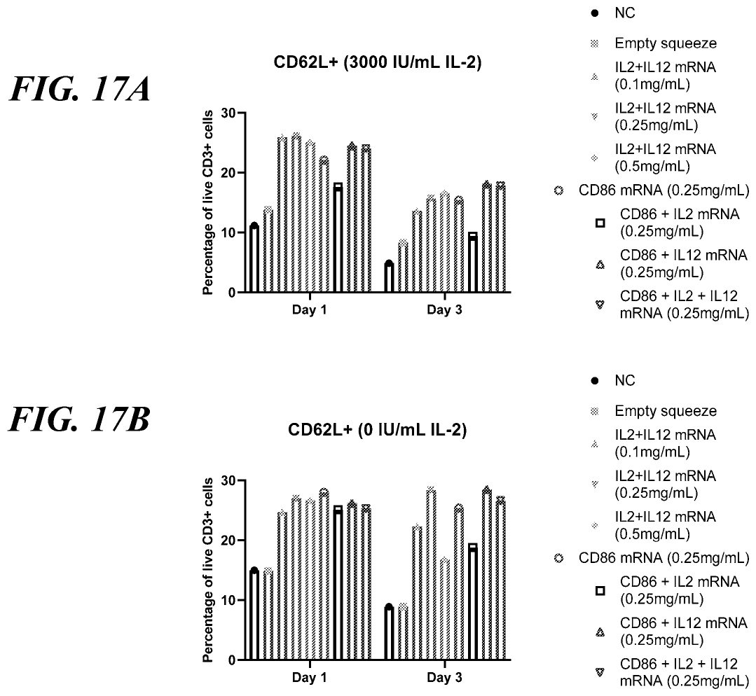

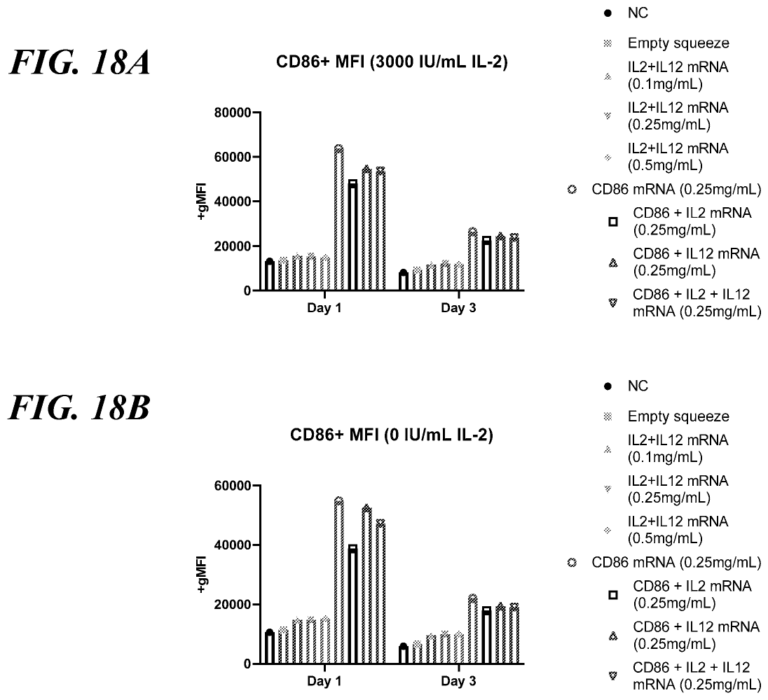

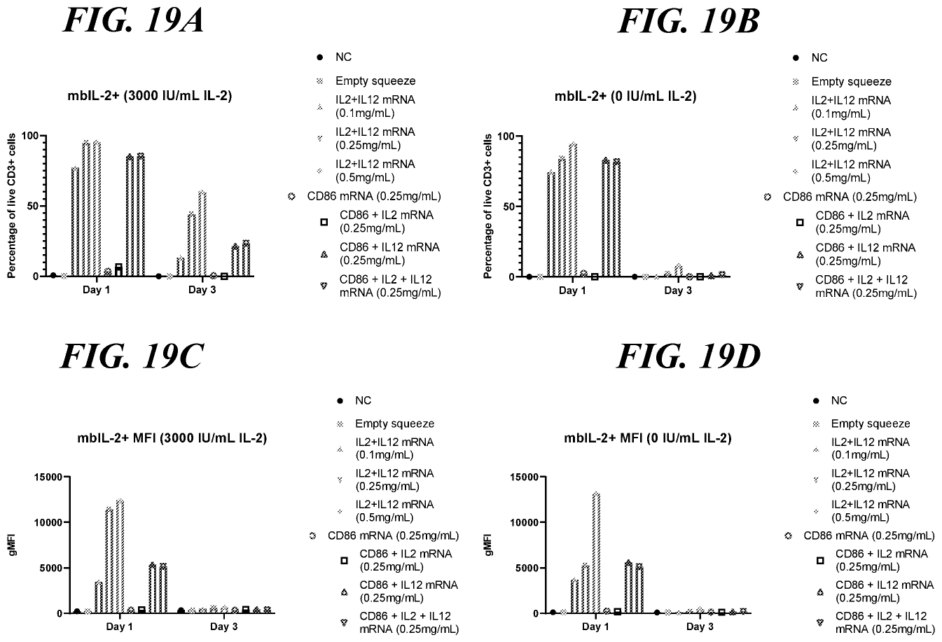

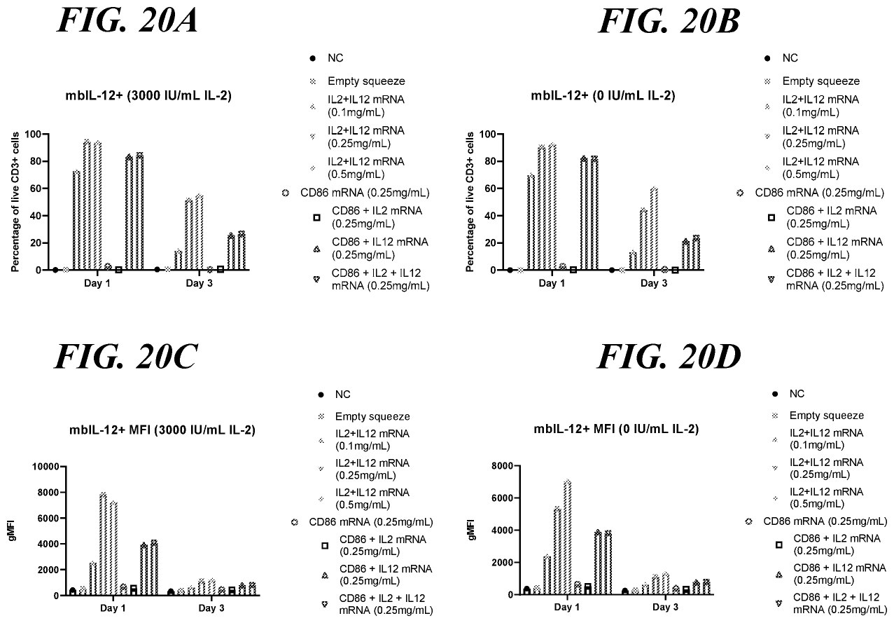

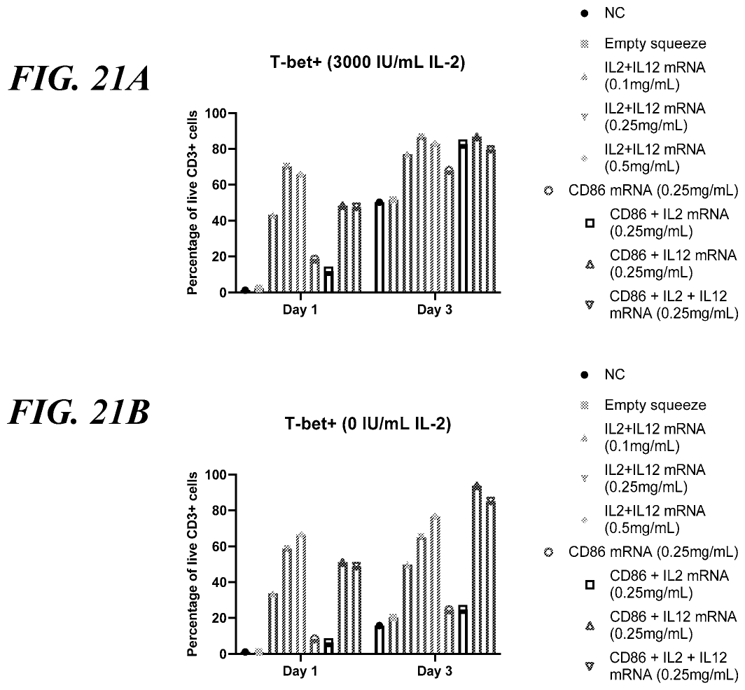

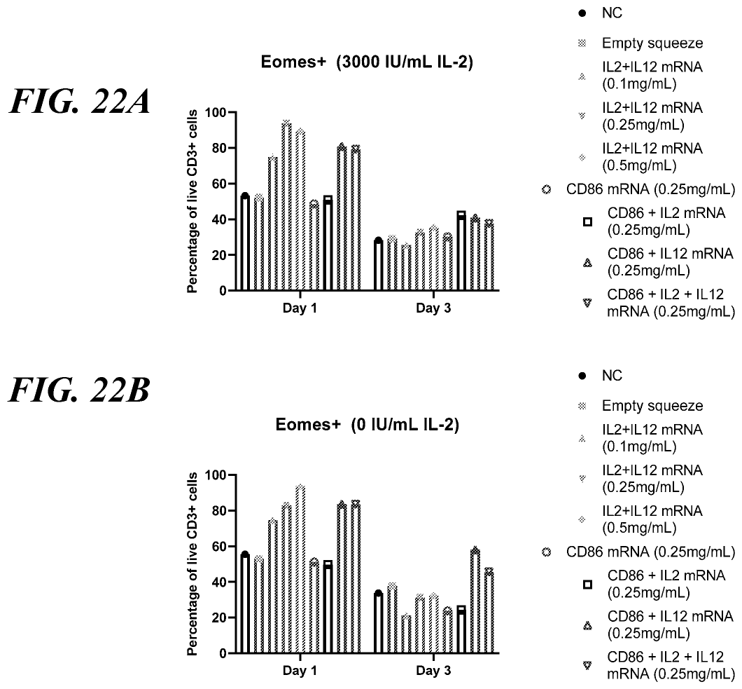

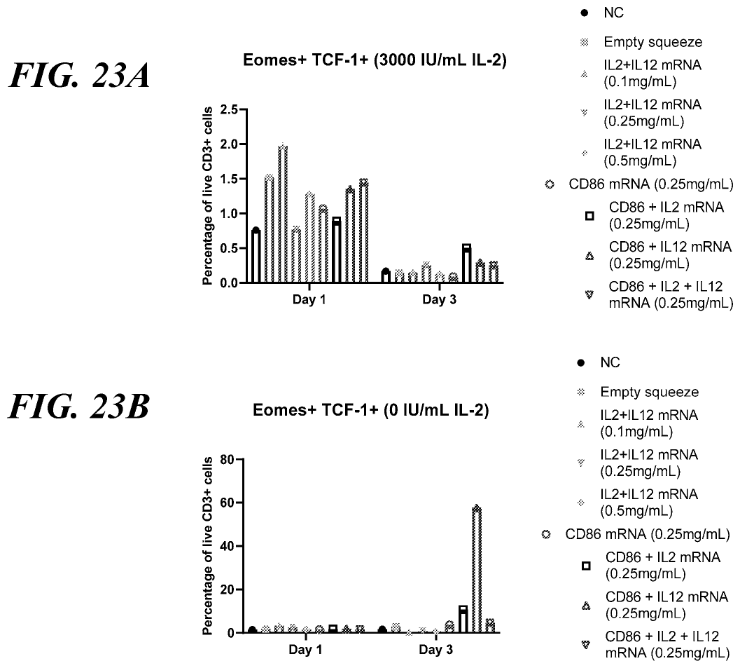

TILs were thawed and cultured in media comprising a high concentration of IL-2 (3000 IU/mL) for 24 hours. Following culture, mRNAs encoding mbIL-2 and mbIL-12 (0.1 mg/mL), mbIL-2 and mbIL-12 (0.25 mg/mL), mbIL-2 and mbIL-12 (0.5 mg/mL), CD86 and mbIL-2 (0.25 mg/mL), CD86 and mbIL-12 (0.25 mg/mL), or CD86 and mbIL-2 and mbIL-12 (0.25 mg/mL each) were delivered to the TILs by squeeze-mediated processing at 30 psi using a ST-10-040-70 chip (10 μm length, 4 μm width, 70 μm depth). The TILs comprising the constriction mediated delivered mRNAs were resuspended in culture media comprising 3000 IU/mL IL-2 or lacking IL-2 for 3 days. Fluorescence-activated cell sorting (FACS) was used to determine the expression of cytokines at 1 day and 3 days following resuspension. Viability of TILs following squeeze-delivery of mRNAs and incubation in the presence of IL-2 is shown in FIGS. 14A and 14B. Proliferation of TILs Viability of TILs following squeeze-delivery of mRNAs and incubation in the presence of IL-2 is shown in FIGS. 15A and 15B. Expression of CD39 on TILs following squeeze-delivery of mRNAs and incubation in the presence of IL-2 is shown in FIGS. 16A and 16B. Expression of CD62L on TILs following squeeze-delivery of mRNAs and incubation in the presence of IL-2 is shown in FIGS. 17A and 17B. FIGS. 18A and 18B show the mean fluorescent intensities of CD86. The percentage of TILs expressing mbIL-2 following squeeze-delivery of mRNAs and incubation in the presence of IL-2 is shown in FIGS. 19A-19D. The percentage of TILs expressing mbIL-12 following squeeze-delivery of mRNAs and incubation in the presence of IL-2 is shown in FIGS. 20A-20D. The percentage of TILs expressing T-bet following squeeze-delivery of mRNAs and incubation in the presence of IL-2 is shown in FIGS. 21A and 21B. The percentage of TILs expressing Eomes following squeeze-delivery of mRNAs and incubation in the presence of IL-2 is shown in FIGS. 22A and 22B. The percentage of TILs expressing Eomes+TCF-1 following squeeze-delivery of mRNAs and incubation in the presence of IL-2 is shown in FIGS. 23A and 23B.

Tables 4 and 5 provide an outline of the FACS protocols used to evaluate the surface and nuclear components of the TILs.

TABLE 4
Flow chart corresponding to the cell surface stain assays from Example 3.

| | Emission | | | | Final |
|---|---|---|---|---|---|
| | (nm) | Channel | Fluorochromes | Target | Dilution |
| Violet | 440/50 | VL-1 | Pacific Blue | IL-12 | 1:200 |
| (405 nm) | 603/28 | VL-3 | BV605 | CD62L | 1:200 |
| Blue | 530/30 | BL-1 | FITC | CD39 | 1:200 |
| (488 nm) | | | | | |
| Yellow | 585/16 | YL-1 | PE | CD3 | 1:200 |
| (561 nm) | 780/60 | YL-4 | PE-Cy7 | CD86 | 1:200 |
| Red | 720/30 | RL-1 | APC | IL-2 | 1:200 |
| (638 nm) | 780/60 | RL-3 | NIR | Live/Dead | 1:1000 |

TABLE 5
Flow chart corresponding to the nuclear stain assays from Example 3.

| | Emission | | | | Final |
|---|---|---|---|---|---|
| | (nm) | Channel | Fluorochromes | Target | Dilution |
| Violet | 440/50 | VL-1 | BV421 | T-bet | 1:200 |
| (405 nm) | | | | | |
| Blue | 695/40 | BL-3 | PerCP/Cy5.5 | CD3 | 1:200 |
| (488 nm) | | | | | |
| Yellow | 585/16 | YL-1 | PE | TCF-1 | 1:200 |
| (561 nm) | | | | | |
| | 670/14 | RL-1 | APC | Eomes | 1:200 |
| Red | 780/60 | RL-3 | NIR | Live/Dead | 1:1000 |
| (638 nm) | | | | | |

### Results

As shown in FIGS. 14A-14B, viability was consistently greater than about 85% in all replicates (e.g., from Day 1 and Day 3) for all cells tested in 3000 IU/mL. The viability of TILs in media lacking IL-2 was greater than about 85% until Day 3 (FIG. 14B). TILs in which mRNA encoding mbIL-2 and mbIL-12 or CD86 mRNA show increased viability in 0 IU/mL IL-2 media. Proliferation increased for TILs in which mRNA encodingmbIL-2 and mbIL-12, and/or CD86 mRNA were delivered using squeeze-mediated delivery (FIGS. 15A-15B).

TILs successfully expressed mbIL-2 and mbIL-12, and CD86, when mRNA was delivered via squeeze-mediated processing as indicated by both percentage of cells expressing the cytokines, and MFI expression (see, e.g., FIGS. 19A-19D and 20A-20D). As shown in FIGS. 19A-19D and 20A-20D, a higher concentration of mRNA encoding mbIL-2 and/or mbIL-12 mRNA (at 0.5 mg/mL) via squeezed-mediated processing showed comparable percentages of cells expressing the respective cytokines, and showed an overall increase in mbIL-2 and/or mbIL-12 expression, respectively, over 3 Days relative to squeeze-mediated processing of 0.25 mg/mL of mRNA encoding mbIL-2 and/or mbIL-12.

The phenotype of TILs receiving mRNAs by squeeze-mediated processing was also evaluated. FIGS. 16A-16B illustrate that CD39 was similar across all TILs, regardless of IL-2 culture conditions. CD62L (central memory T-cells) expression was improved in the presence of mbIL-12 mRNA (FIGS. 17A-17B). CD86 MFI was similarly improved in the presence of mbIL-12 mRNA in TILs (FIGS. 18A-18B). Eomes+ and TCF-1+ expression was enhanced in TILs that received mRNAs with greater concentrations of IL-2 in the culture media (FIGS. 23A-23B).

### Example 4: Tumor Infiltrating Lymphocytes (TILs) Express Membrane Bound Cytokines Up to 72 Hours Subsequent to Constriction Mediated Delivery of mRNAs Encoding Membrane-Bound Cytokines without Additional Exogenous rhIL2

This example shows the expression of membrane-bound IL-2 (e.g., mbIL-2) and membrane-bound IL-12 (e.g., mbIL-12) in TILs over 72 hours, subsequent to constriction mediated delivery of mbIL-2 and mbIL-12 mRNA.

### Methods

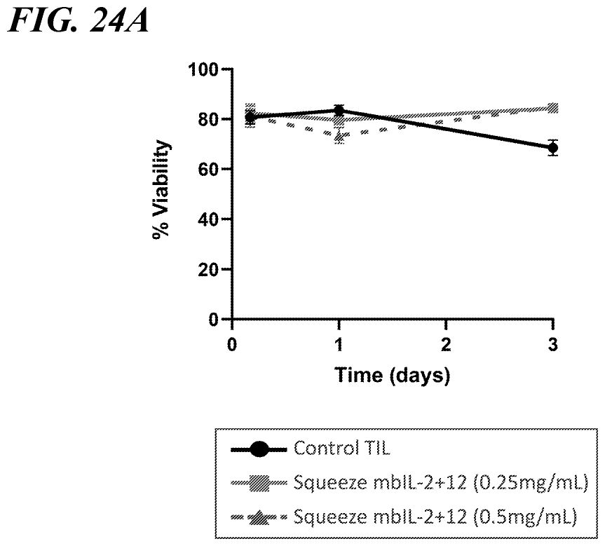

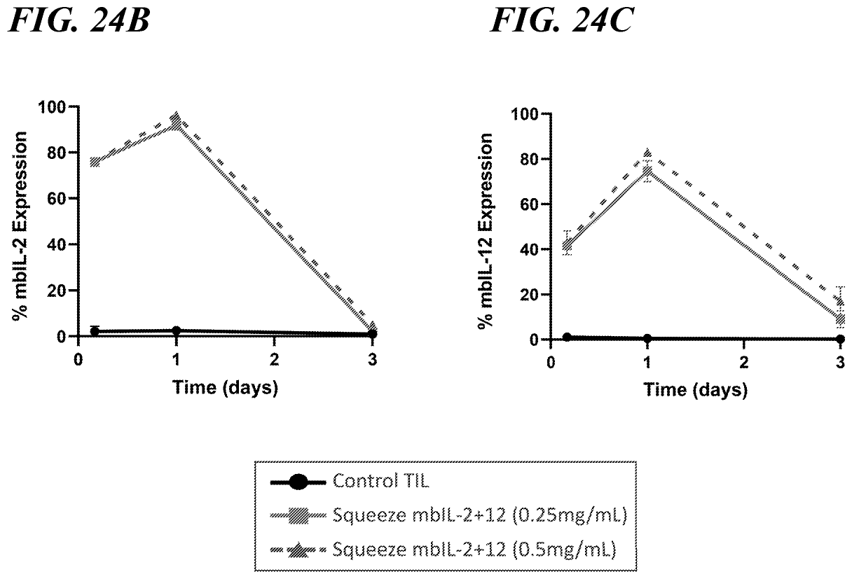

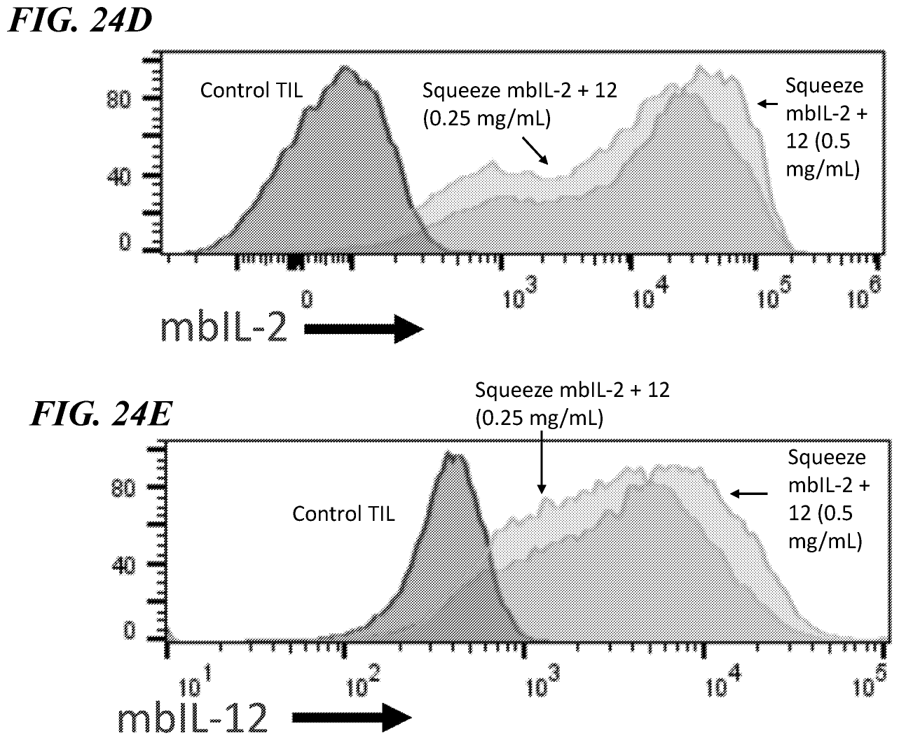

TILs were thawed and cultured in media comprising a concentration of IL-2 (3000 IU/mL) for 24 hours. Following culture, two groups: (1) mRNAs encoding mbIL-2 and mbIL-12 (total 0.25 mg/mL) and (2) mRNAs encoding mbIL-2 and mbIL-12 (total 0.50 mg/mL) were delivered to the TILs by constriction mediated delivery (“squeezing”) respectively. TILs were squeeze-processed at 30 psi using a ST-10-04-70 chip (10 μm length, 4 μm width, 70 μm depth) in the presence of the mRNAs as described. Constriction mediated delivery was likewise performed with empty payload to serve as controls for downstream analysis (Control TIL). The TILs comprising the squeeze-delivered mRNAs were resuspended in culture media without exogenous rhIL-2, and cultured for 3 days. Viability and expression of mbIL-2 and mbIL-12 were quantified by flow cytometry. (FIGS. 24A-24E).

### Results

As shown in FIG. 24A, viability of modified TILs comprising squeeze-delivered mbIL-2 and mbIL-12 mRNAs was consistently high (around 80%) at all measured time points post-squeezing (Days 0, 1, 2, and 3).

As shown in FIGS. 24B-E, The mRNA encoding mbIL-2 and mbIL-12 were effectively delivered simultaneously into TILs using the constricted mediated delivery method and subsequently expressed.

### Example 5: TILs Squeeze-Loaded with mRNAs Encoding Membrane-Bound Cytokines Proliferate in Tumor Co-Culture

This example shows the analysis of proliferation of TILs comprising squeeze-loaded mbIL-2 and mbIL-12 RNAs at 3 days as analyzed by the number of viable CD8+ T Cells in each sample, Ki67 expression by flow cytometry, as well as Granzyme B expression by flow cytometry upon co-culture with tumor cells.

### Methods

TILs were isolated from 3 melanoma patients' tumor tissue. The TILs were thawed and cultured overnight in rhIL-2-free media. Following culture, two groups: (1) mRNAs encoding mbIL-2 and (2) mRNAs encoding mbIL-2 and mbIL-12 were delivered to the TILs by constriction mediated delivery (“squeezing”) respectively. TILs were squeeze-processed at 30 psi using a ST-10-04-70 chip (10 μm length, 4 μm width, 70 μm depth) in the presence of the mRNAs as described. Constriction mediated delivery was likewise performed with empty payload to serve as controls for downstream analysis (Control TIL). The squeeze-processed TILs were then cultured for 3 days in the presence of autologous tumor cells without exogenous cytokines. As an additional control, TILs squeezed with empty payload (Control TIL) were cultured with autologous tumor cells in media containing 3000 IU/mL rhIL-2 and 10 ng/mL rh IL-12.

### Results

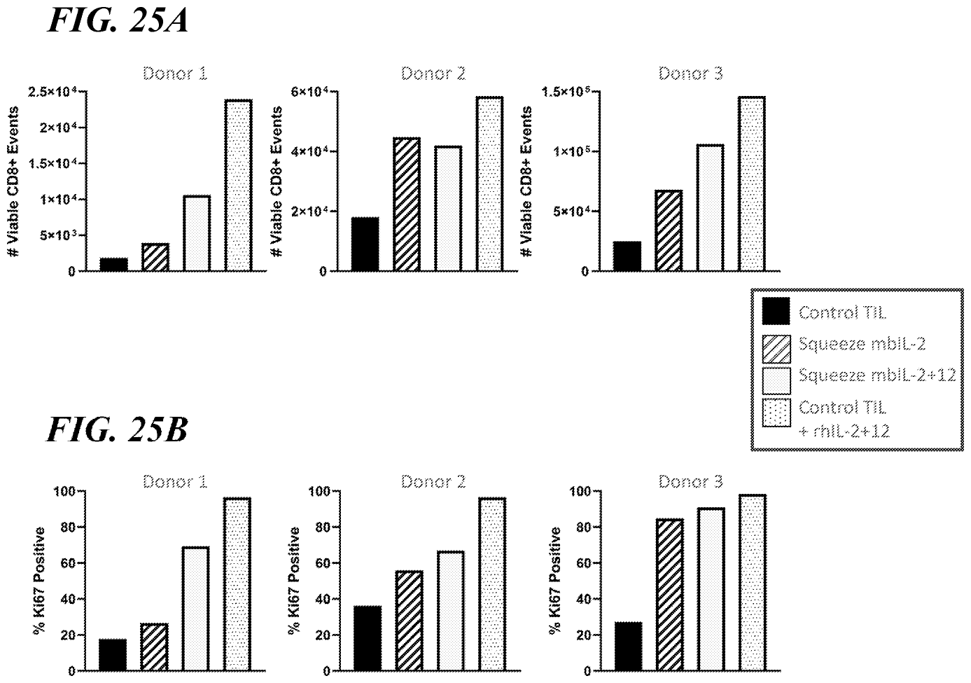

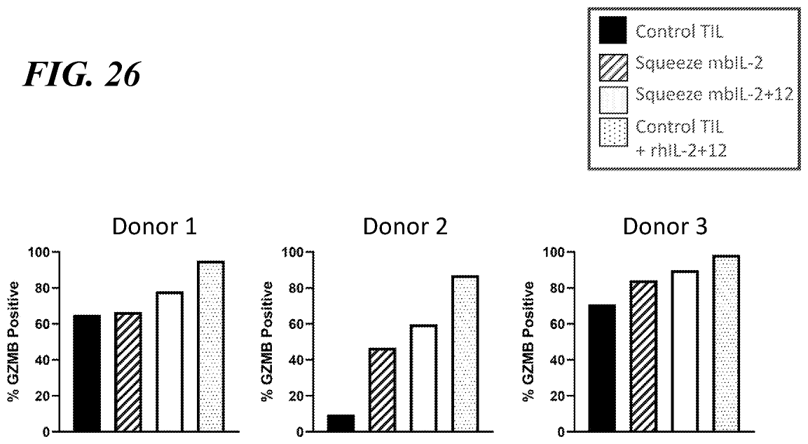

As shown in FIG. 25A, TILs squeezed with mbIL2 mRNA only, or with mbIL-2 and mbIL-12 mRNAs cultured in the presence of autologous tumor cells without exogenous IL-2 or IL-12 showed increased cell proliferation, as measured by the number of CD8+ T cells in each donor sample, as compared to TILs squeezed with empty payload (Control TIL) for all three donor samples. As shown in FIG. 25B, TILs squeezed with mbIL2 mRNAs only, or with mbIL-2 and mbIL-12 mRNAs cultured in the presence of autologous tumor cells without exogenous IL-2 or IL-12 showed upregulation of Ki67 and an increased percentage of Ki67 positive cells in each sample, as compared to TILs squeezed with empty payload (Control TIL) for all three donor samples. As shown in FIG. 26, the TILs were analyzed for Granzyme B expression by flow cytometry after 20 hours of co-culture with autologous tumor cells and similarly showed upregulation of Granzyme B in TILs squeezed with (1) mbIL2 mRNAs only, or with (2) mbIL-2 and mbIL-12 mRNAs upon co-culture with autologous tumor cells in the absence of exogenous IL-2 or IL-12, as compared to Control TIL.

### Example 6: Tumor Killing by TILs Squeeze-Loaded with mRNAs Encoding Membrane-Bound Cytokines when Co-Cultured with Autologous Tumor Cells

This example shows tumor killing by TILs squeezed with (1) mbIL-2 mRNA or (2) mbIL-2 and mbIL-12 mRNAs in co-culture with tumor cells the absence of exogenous cytokines, as compared to that of control TILs co-cultured with tumor cells in the presence of exogenous rhIL-2 and rhIL-12.

### Methods

TILs were thawed and cultured for 1-2 days in cytokine-free media with a 2:1 ratio of autologous tumor cell lines stained with Cytolight Red and imaged for 24 hours using an IncuCyte fluorescent microscope. Following culture, two groups: (1) mRNAs encoding mbIL-2 and (2) mRNAs encoding mbIL-2 and mbIL-12 were delivered to the TILs by constriction mediated delivery (“squeezing”), respectively. TILs were squeeze-processed at 30 psi using a ST-10-04-70 chip (10 μm length, 4 μm width, 70 μm depth) in the presence of the mRNAs as described. Constriction mediated delivery was likewise performed with empty payload to serve as controls for downstream analysis (Control TIL). The squeeze-processed TILs were then co-cultured with autologous tumor cells in the absence of exogenous cytokines. As an additional control, TILs squeezed with empty payload (Control TIL) were cultured with autologous tumor cells in media containing 3000 IU/mL rhIL-2 and 10 ng/mL rh IL-12. A red mask was applied to the IncuCyte images and used to calculate the percentage of tumor confluence.

### Results

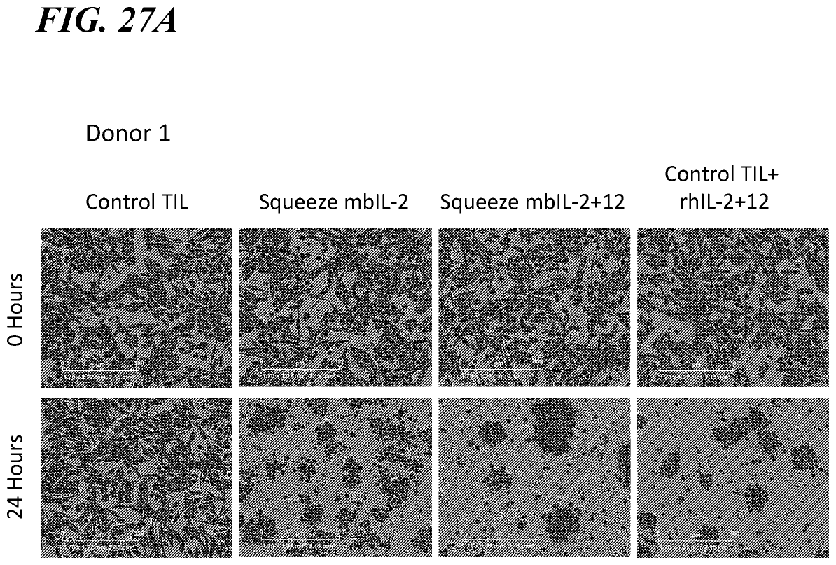

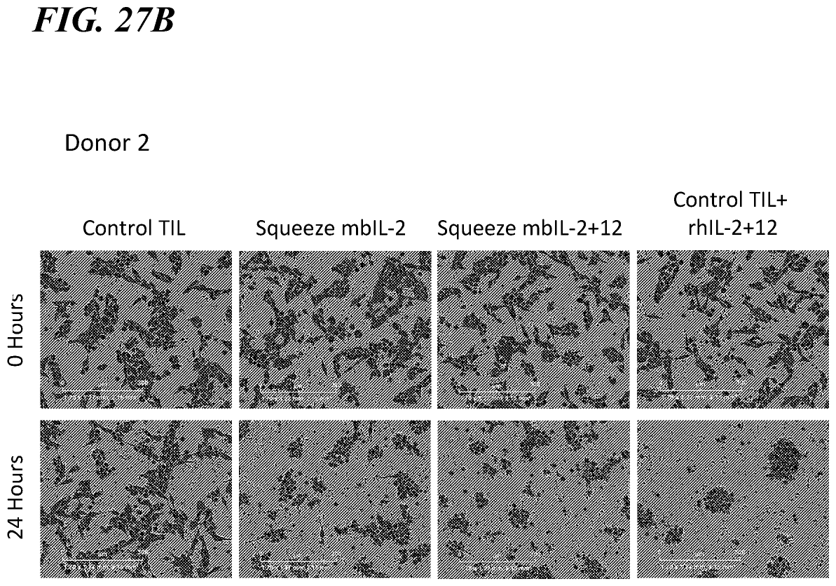

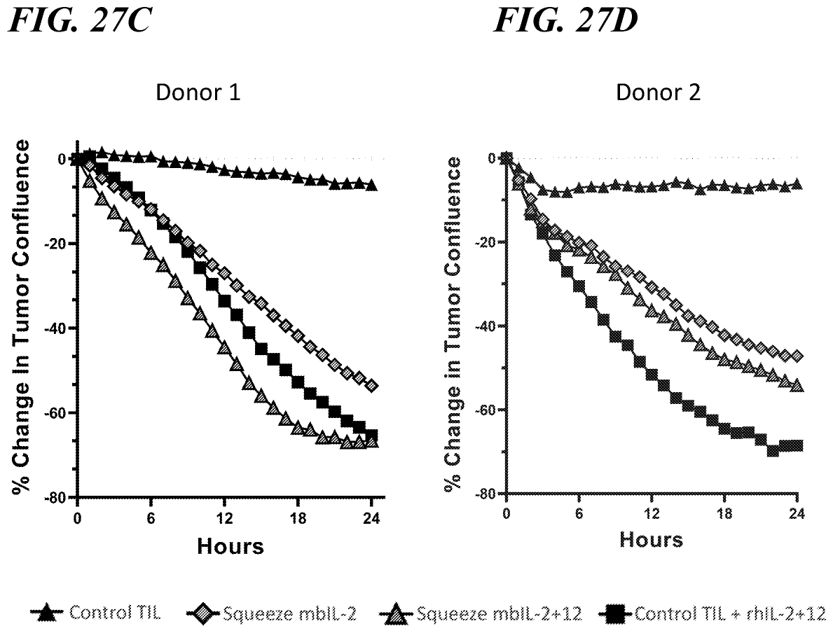

As shown in FIGS. 27A-D, TILs squeezed with (1) mbIL-2 mRNA or with (2) mbIL-2 and mbIL-12 mRNAs, show comparable tumor killing capability upon co-culture in the absence of exogenous cytokines, as compared to that of control TILs in co-culture with tumor cells while supported by exogenous rhIL-2 and rhIL-12. TILs squeezed with (1) mbIL-2 mRNA or with (2) mbIL-2 and mbIL-12 mRNAs, also show significantly enhanced tumor killing capability upon co-culture in the absence of exogenous cytokines as compared to that of control TILs in co-culture in the absence of exogenous cytokines.

Sequence Listing

| | | |
|---|---|---|
| 1 | ATGATGGTGGACGGCGACAACAGCCACGTGGAAATGAAG | TFRC_G4S_IFN- |
| | CTGGCCGTGGACGAGGAAGAGAACGCCGACAACAACAC | a2a mRNA |
| | CAAGGCCAACGTGACCAAGCCTAAGAGATGCAGCGGCAG | |
| | CATCTGCTACGGCACAATCGCCGTGATCGTGTTCTTCCTG | |
| | ATCGGCTTTATGATCGGCTACCTGGGCTACTGCAAGAGCA | |
| | GTGATGGACCTGGCGAAACAGGCGGAGGCGGAGGATCTG | |
| | GTGGCGGAGGAAGCGGTGGCGGCGGATCTTGTGATCTGC | |
| | CTCAGACACACAGCCTGGGCAGCAGACGAACACTGATGC | |
| | TGCTGGCCCAGATGCGGAAGATCAGCCTGTTCAGCTGCCT | |
| | GAAGGACCGGCACGATTTCGGCTTCCCTCAAGAGGAATT | |
| | CGGCAACCAGTTCCAGAAGGCCGAGACAATCCCTGTGCT | |
| | GCACGAGATGATCCAGCAGATCTTCAACCTGTTCTCCACC | |
| | AAGGACAGCAGCGCCGCCTGGGATGAGACACTGCTGGAC | |
| | AAGTTCTACACCGAGCTGTACCAGCAGCTGAATGACCTG | |
| | GAAGCCTGCGTGATCCAAGGCGTGGGAGTGACAGAGACA | |
| | CCCCTGATGAAGGAAGATAGCATCCTGGCCGTGCGCAAG | |
| | TACTTCCAGCGGATCACCCTGTACCTGAAAGAGAAGAAG | |
| | TACAGCCCCTGCGCCTGGGAAGTCGTGCGGGCCGAAATC | |
| | ATGAGAAGCTTCAGCCTGAGCACCAACCTGCAAGAGAGC | |
| | CTGCGGAGCAAAGAGTGA | |
| 2 | ATGATGGTGGACGGCGACAACAGCCACGTGGAAATGAAG | TFRC_G4S_IL-12 |
| | CTGGCCGTGGACGAGGAAGAGAACGCCGACAACAACAC | mRNA |
| | CAAGGCCAACGTGACCAAGCCTAAGAGATGCAGCGGCAG | |
| | CATCTGCTACGGCACAATCGCCGTGATCGTGTTCTTCCTG | |
| | ATCGGCTTTATGATCGGCTACCTGGGCTACTGCAAGAGCA | |
| | GTGATGGACCTGGCGAAACAGGCGGAGGCGGAGGATCTG | |
| | GTGGCGGAGGAAGTGGCGGCGGAGGTTCTATTTGGGAGC | |
| | TGAAGAAAGACGTGTACGTGGTGGAACTGGACTGGTATC | |
| | CCGATGCTCCTGGCGAGATGGTGGTGCTGACCTGCGATA | |
| | CCCCTGAAGAGGACGGCATCACCTGGACACTGGATCAGT | |
| | CTAGCGAGGTGCTCGGCAGCGGCAAGACCCTGACCATCC | |
| | AAGTGAAAGAGTTTGGCGACGCCGGCCAGTACACCTGTC | |
| | ACAAAGGCGGAGAAGTGCTGAGCCACAGCCTGCTGCTGC | |
| | TCCACAAGAAAGAGGATGGCATTTGGAGCACCGACATCC | |
| | TGAAGGACCAGAAAGAGCCCAAGAACAAGACCTTCCTGA | |
| | GATGCGAGGCCAAGAACTACAGCGGCCGGTTCACATGTT | |
| | GGTGGCTGACCACCATCAGCACCGACCTGACCTTCAGCG | |
| | TGAAGTCCAGCAGAGGCAGCAGTGATCCTCAGGGCGTTA | |
| | CATGTGGCGCCGCTACACTGTCTGCCGAAAGAGTGCGGG | |
| | GCGATAACAAAGAATACGAGTACAGCGTGGAATGCCAAG | |
| | AGGACAGCGCCTGTCCAGCCGCCGAAGAGTCTCTGCCTA | |
| | TCGAAGTGATGGTCGACGCCGTGCACAAGCTGAAGTACG | |
| | AGAACTACACCAGCAGCTTTTTCATCCGGGACATCATCAA | |
| | GCCCGATCCTCCAAAGAACCTGCAGCTGAAGCCTCTGAA | |
| | GAACAGCAGACAGGTGGAAGTGTCCTGGGAGTACCCCGA | |
| | CACCTGGTCTACACCCCACAGCTACTTCAGCCTGACCTTT | |
| | TGCGTGCAAGTGCAGGGCAAGTCCAAGCGCGAGAAAAA | |
| | GGACCGGGTGTTCACCGACAAGACCAGCGCCACCGTGAT | |
| | CTGCAGAAAGAACGCCAGCATCAGCGTCAGAGCCCAGGA | |
| | CCGGTACTACAGCAGCTCTTGGAGCGAATGGGCCAGCGT | |
| | GCCATGTAGCGGAGGTGGTGGTAGCGGAGGCGGCGGAA | |
| | GCGGCGGTGGTGGATCAGGTGGTGGTGGCTCTAGAAACC | |
| | TGCCAGTGGCTACCCCTGATCCTGGCATGTTCCCTTGTCT | |
| | GCACCACAGCCAGAACCTGCTGAGAGCCGTGTCCAACAT | |
| | GCTGCAGAAGGCCAGACAGACCCTGGAATTCTACCCCTG | |
| | CACCAGCGAGGAAATCGACCACGAGGACATCACCAAGG | |
| | ATAAGACCAGCACCGTGGAAGCCTGCCTGCCTCTGGAAC | |
| | TGACCAAGAACGAGAGCTGCCTGAACAGCCGGGAAACCT | |
| | CCTTCATCACCAACGGCTCTTGCCTGGCCAGCAGAAAGA | |
| | CAAGCTTCATGATGGCCCTGTGCCTGAGCAGCATCTACGA | |
| | GGACCTGAAGATGTACCAGGTGGAATTCAAGACCATGAA | |
| | CGCCAAGCTGCTGATGGACCCCAAGCGGCAGATCTTCCT | |
| | GGACCAGAACATGCTGGCTGTGATCGACGAGCTGATGCA | |
| | GGCCCTGAACTTCAACAGCGAGACAGTGCCCCAGAAGTC | |
| | TAGCCTGGAAGAACCCGACTTCTACAAGACCAAGATCAA | |
| | GCTGTGCATCCTGCTGCACGCCTTCCGGATCAGAGCCGTG | |
| | ACCATCGACAGAGTGATGAGCTACCTGAACGCCTCCTGA | |
| 3 | (G₄S)₃ | G₄S Linker |
| 4 | (EAAAK)₃ | EAAAK linker |
| 5 | (G₄S)ₙ | G₄S Linker |
| 6 | (EAAAK)n | EAAAK linker |
| 7 | MMVDGDNSHVEMKLAVDEEENADNNTKANVTKPKRCSGS | TFRC_G4S_IL-2 |
| | ICYGTIAVIVFFLIGFMIGYLGYCKSSDGPGETG | |
| | GGGGSGGGGSGGGGS | |
| | APTSSSTKKTQLQLEHLLLDLQMILNGINNYKNPKLTRMLTF | |
| | KFYMPKKATELKHLQCLEEELKPLEEVLNLAQSKNFHLRPR | |
| | DLISNINVIVLELKGSETTFMCEYADETATIVEFLNRWITFCQ | |
| | SIISTL | |
| 8 | MLKKRGNHSTGLCLLVMFFMVLVALVGLGLGMFQLFHLQ | FasL_G4S_IL-2 |
| | KETGGGGGSGGGGSGGGGS | |
| | APTSSSTKKTQLQLEHLLLDLQMILNGINNYKNPKLTRMLTF | |
| | KFYMPKKATELKHLQCLEEELKPLEEVLNLAQSKNFHLRPR | |
| | DLISNINVIVLELKGSETTFMCEYADETATIVEFLNRWITFCQ | |
| | SIISTLT | |
| 9 | MMVDGDNSHVEMKLAVDEEENADNNTKANVTKPKRCSGS | TFRC_G4S_IL-12 |
| | ICYGTIAVIVFFLIGFMIGYLGYCKSSDGPGETG | |
| | GGGGSGGGGSGGGGS | |
| | IWELKKDVYVVELDWYPDAPGEMVVLTCDTPEEDGITWTL | |
| | DQSSEVLGSGKTLTIQVKEFGDAGQYTCHKGGEVLSHSLLL | |
| | LHKKEDGIWSTDILKDQKEPKNKTFLRCEAKNYSGRFTCW | |
| | WLTTISTDLTFSVKSSRGSSDPQGVTCGAATLSAERVRGDN | |
| | KEYEYSVECQEDSACPAAEESLPIEVMVDAVHKLKYENYTS | |
| | SFFIRDIIKPDPPKNLQLKPLKNSRQVEVSWEYPDTWSTPHS | |
| | YFSLTFCVQVQGKSKREKKDRVFTDKTSATVICRKNASISVR | |
| | AQDRYYSSSWSEWASVPCSGGGGSGGGGSGGGGSGGGGSR | |
| | NLPVATPDPGMFPCLHHSQNLLRAVSNMLQKARQTLEFYPC | |
| | TSEEIDHEDITKDKTSTVEACLPLELTKNESCLNSRETSFITN | |
| | GSCLASRKTSFMMALCLSSIYEDLKMYQVEFKTMNAKLLM | |
| | DPKRQIFLDQNMLAVIDELMQALNFNSETVPQKSSLEEPDFY | |
| | KTKIKLCILLHAFRIRAVTIDRVMSYLNAS | |
| 10 | MMVDGDNSHVEMKLAVDEEENADNNTKANVTKPKRCSGS | TFRC_G4S_IFN- |
| | ICYGTIAVIVFFLIGFMIGYLGYCKSSDGPGETG | a2a |
| | GGGGSGGGGSGGGGS | |
| | CDLPQTHSLGSRRTLMLLAQMRKISLFSCLKDRHDFGFPQEE | |
| | FGNQFQKAETIPVLHEMIQQIFNLFSTKDSSAAWDETLLDKF | |
| | YTELYQQLNDLEACVIQGVGVTETPLMKEDSILAVRKYFQR | |
| | ITLYLKEKKYSPCAWEVVRAEIMRSFSLSTNLQESLRSKE | |
| 11 | MMVDGDNSHVEMKLAVDEEENADNNTKANVTKPKRCSGS | TFRC domain |
| | ICYGTIAVIVFFLIGFMIGYLGYCKSSDGPGETG | |
| 12 | MLKKRGNHSTGLCLLVMFFMVLVALVGLGLGMFQLFHLQ | FasL domain |
| | KETG | |

1. A method of increasing Granzyme B expression on tumor-infiltrating lymphocytes (TILs), comprising: (a) modifying the TILs to increase expression of: (i) a co-stimulatory molecule, (ii) a cytokine, or (ii) both a co-stimulatory molecule and a cytokine; and (b) culturing the TILs in the absence of an exogenous cytokine, wherein after the modifying and the culturing, the TILs exhibit increased Granzyme B expression upon activation, as compared to that of corresponding TILs that have not been modified (reference TILs).

2. (canceled)

3. The method of claim 1, wherein the Granzyme B expression is increased by at least about 1.5-fold, at least about 2-fold, at least about 2.5-fold, at least about 3-fold, at least about 3.5 fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, as compared to that of the reference TILs.

4. The method of claim 1, wherein the culturing is for at least about one day, at least about two days, or at least about three days.

5. The method of claim 1, wherein the co-stimulatory molecule is CD86.

6. The method of claim 1, wherein the cytokine comprises a membrane-bound IL-2, membrane-bound IL-12, or both.

7. The method of claim 1, wherein the modifying comprises passing a cell suspension comprising the TILs through a cell-deforming constriction, thereby causing perturbations of the TILs such that a nucleic acid encoding the co-stimulatory molecule and/or a nucleic acid encoding the cytokine enters the TILs through the perturbations when contacted with the TILs.

8. The method of claim 7, wherein the modifying further comprises contacting the TILs with the nucleic acid encoding the co-stimulatory molecule and/or the nucleic acid encoding the cytokine.

9. The method of claim 1, wherein the TILs were modified by passing a cell suspension comprising input TILs through a cell-deforming constriction, thereby causing a perturbation of the inputs TILs such that a nucleic acid encoding the co-stimulatory molecule and/or a nucleic acid encoding the cytokine entered the input TILs when contacted with the TILs, wherein after the nucleic acid encoding the co-stimulatory molecule and/or the nucleic acid encoding the cytokine, the input TILs exhibit increased expression of the co-stimulatory molecule and/or cytokine to become the TILs that have been modified.

10. The method of claim 9, further comprising contacting the cell suspension with the nucleic acid encoding the co-stimulatory molecule and/or the nucleic acid encoding the cytokine.

11. The method of claim 10, wherein the cell suspension is contacted with the nucleic acid encoding the co-stimulatory molecule and/or the nucleic acid encoding the cytokine
a) prior to the passing of the cell suspension through the cell-deforming constriction; and/or
b) during the passing of the cell suspension through the cell-deforming constriction.

12-13. (canceled)

14. The method of claim 7, wherein the nucleic acid encoding the co-stimulatory molecule and/or the nucleic acid encoding the cytokine is mRNA.

15. The method of claim 7, wherein the cell-deforming constriction comprises a width, which is about 10% to about 99% of the mean diameter of the TILs.

16. The method of claim 15, wherein the cell-deforming constriction comprises a width, which is about 10% to about 99% of the mean diameter of the input TILs.

17. The method of claim 16, wherein the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm.

18. The method of claim 16 or 17, wherein the width of the constriction is about 3 μm to about 5 μm.

19. The method of claim 18, wherein the width of the constriction is about 4 μm.

20. A method of increasing Granzyme B expression on tumor-infiltrating lymphocytes (TILs), comprising culturing the TILs in the absence of exogenous cytokine, wherein the TILs have been modified to increase expression of: (i) a co-stimulatory molecule, (ii) a cytokine, or (iii) both a co-stimulatory molecule and a cytokine, and wherein after the culturing, the TILs exhibit increased proliferation upon activation, as compared to that of corresponding TILs that have not been modified (reference TILs).

21. The method of claim 20, wherein the TILs were modified by passing a cell suspension comprising input TILs through a cell-deforming constriction, thereby causing a perturbation of the inputs TILs such that a nucleic acid encoding the co-stimulatory molecule and/or a nucleic acid encoding the cytokine entered the input TILs when contacted with the TILs, wherein after the nucleic acid encoding the co-stimulatory molecule and/or the nucleic acid encoding the cytokine, the input TILs exhibit increased expression of the co-stimulatory molecule and/or cytokine to become the TILs that have been modified.

22. The method of claim 21, wherein the cell suspension is contacted with the nucleic acid encoding the co-stimulatory molecule and/or the nucleic acid encoding the cytokine
a) prior to the passing of the cell suspension through the cell-deforming constriction; and/or
b) during the passing of the cell suspension through the cell-deforming constriction.

23. The method of claim 21, wherein the cell-deforming constriction comprises a width and wherein the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm.
